(19) United States
(12) Patent Application Publication
(10) Pub. No.: US 2024/0294876 A1
(43) Pub. Date: Sep. 5, 2024

(54) METHODS FOR THE EXPANSION OF HUMAN GRANULOCYTE-MACROPHAGE PROGENITORS AND APPLICATIONS THEREOF

(71) Applicant: University of Southern California, Los Angeles, CA (US)

(72) Inventors: Qi-Long Ying, Los Angeles, CA (US); Zheng Guo, Los Angeles, CA (US); Shi Yue, Los Angeles, CA (US); Tai Nguyen, Los Angeles, CA (US); Jiaqi Tang, Los Angeles, CA (US); Chao Zhang, Los Angeles, CA (US)

(21) Appl. No.: 18/561,900

(22) PCT Filed: May 18, 2022
(86) PCT No.: PCT/US2022/029878

§ 371 Date: Nov. 17, 2023

Related U.S. Application Data
(60) Provisional application No. 63/190,103, filed on May 18, 2021.

(51) Int. Cl. C12N 5/0789

(52) U.S. Cl. CPC C12N 5/0647; C12N 2501/125; C12N 2501/999; C12N 2506/11; C12N 2510/00

(57) ABSTRACT

The disclosure provides methods for the long-term expansion of granulocyte-macrophage progenitors, the granulocyte-macrophage progenitors generated therefrom, and uses of the granulocyte-macrophage progenitors thereof.

### CROSS REFERENCE TO RELATED APPLICATIONS

This application claims priority under 35 U.S.C. § 119 from Provisional Application Ser. No. 63/190,103, filed May 18, 2021, the disclosures of which are incorporated herein by reference.

### TECHNICAL FIELD

The disclosure provides methods for the expansion of granulocyte-macrophage progenitors, the granulocyte-macrophage progenitors generated therefrom, and uses of the granulocyte-macrophage progenitors thereof.

### BACKGROUND

Granulocytes, macrophages, and dendritic cells are the essential components of the innate immune system in humans. They are the first line of defense against pathogens and also play a central role in maintaining the homeostasis of our body and preventing various diseases including infection, metabolic diseases and cancer. These cells originate from a common progenitor in the bone marrow, the granulocyte-macrophage progenitor (GMP).

### SUMMARY

Provided herein are methods that promote the expansion of granulocyte/macrophage progenitors (GMPs), e.g., the long-term and clonal expansion of GMPs. These methods are generally applicable in generating long-term and clonal expansion of GMPs from any number of subject sources, including from mice, rats, humans, etc. In some embodiments, one of the many advantages of the GMPs generated by the methods of the disclosure, is that the GMPs are susceptible to genetic modification techniques, thereby allowing for the use of the GMPs in basic scientific research and clinical therapeutic applications. Thus, expanded and genetically modified GMPs can be readily translated into broad clinical applications. For example, human GMPs can be genetically modified so that they differentiate into macrophages (e.g., knockout SIRPα and/or PI3Kγ gene). These engineered macrophages may or are expected to have enhanced antitumor effects and can be used clinically to treat cancer, either as monotherapy or combination therapy with other immunological agents, such as anti-PD-1/PD-L1 antibodies and chimeric antigen receptor T (CAR-T) cells. In addition, ex vivo expanded human GMPs can be readily used for infusion or transplantation to treat neutropenia cause by, for example, chemotherapy, radiotherapy and the like. Such ex vivo expanded GMPs can be autologous or allogeneic to the subject.

The disclosure provides a method for the expansion of a population of granulocyte/macrophage progenitor cells (GMPs) in a culture medium comprising: (i) a growth factor; (ii) a B-Raf kinase inhibitor; and (iii) a compound having the structure of Formula I:

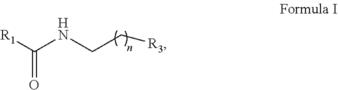

wherein, R¹ is selected from:

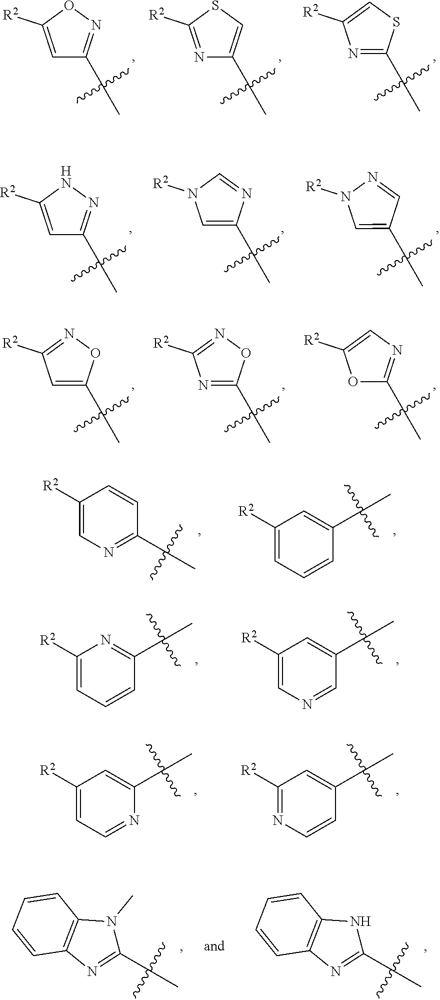

R² is selected from:

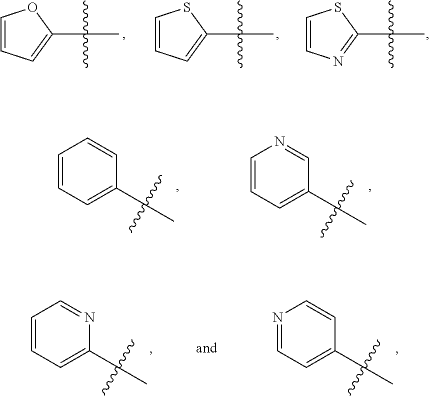

R³ is selected from:

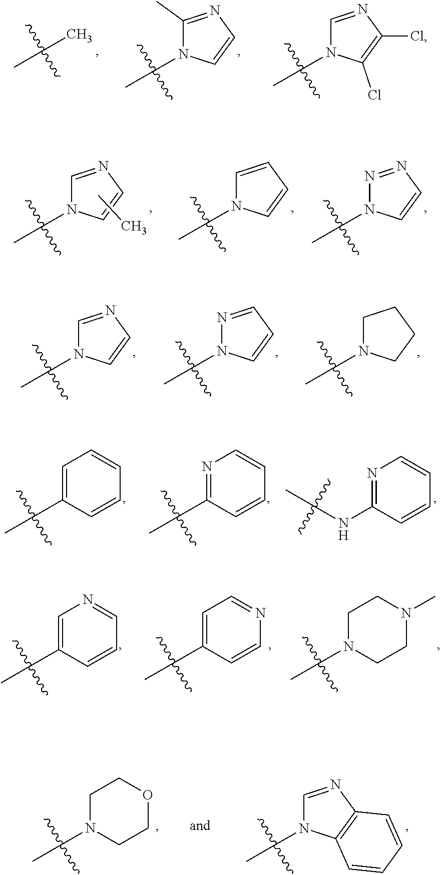

n is an integer selected from 0, 1, 2, 3, 4, and 5; wherein the GMPs remain substantially morphologically unchanged after undergoing multiple cell passages and/or clonal expansion. In one embodiment, the GMPs are derived or obtained from stem cells. In a further embodiment, the stem cells are genetically engineered prior to or during culturing. In yet another or further embodiment, the stem cells are hematopoietic stem cells. In yet another or further embodiment, the hematopoietic stem cells are isolated from the bone marrow of a subject. In a further embodiment, the subject is a mammalian subject. In yet another or further embodiment, the subject is a human, a rat or a mouse. In yet another or further embodiment, the culture medium comprises DMEM/F12 and Neural Basal Medium. In yet another or further embodiment, the culture medium comprises DMEM/F12 and Neural Basal Medium in a ratio of about 5:1 to about 1:5. In yet another or further embodiment, the culture medium comprises DMEM/F12 and Neural Basal Medium in a ratio of about 1:1. In yet another or further embodiment, the culture medium comprises one or more supplements selected from insulin, transferrin, BSA fraction V, putrescine, sodium selenite, DL-α tocopherol, linolenic acid and/or linoleic acid. In yet another or further embodiment, the culture medium is supplemented with insulin, transferrin, BSA fraction V, putrescine, sodium selenite, DL-α tocopherol, and linolenic acid and/or linoleic acid. In a certain embodiment, the compound having the structure of Formula I is selected from:

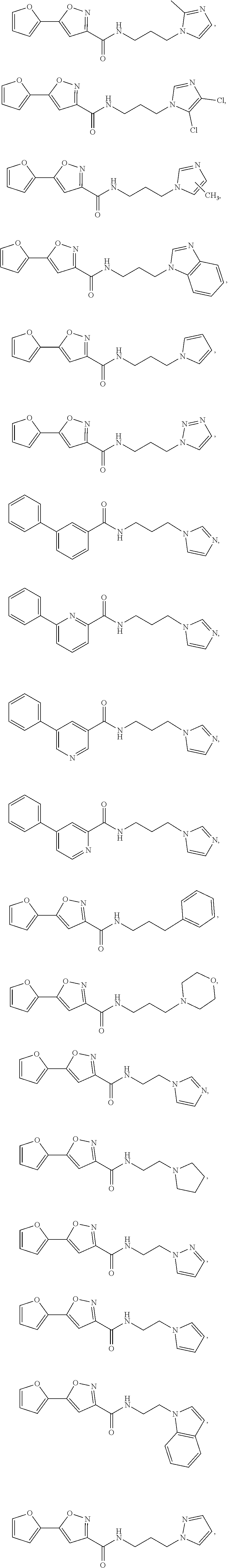

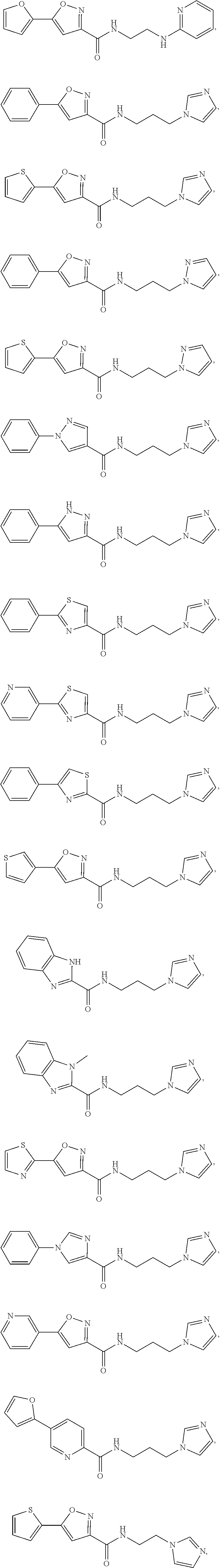

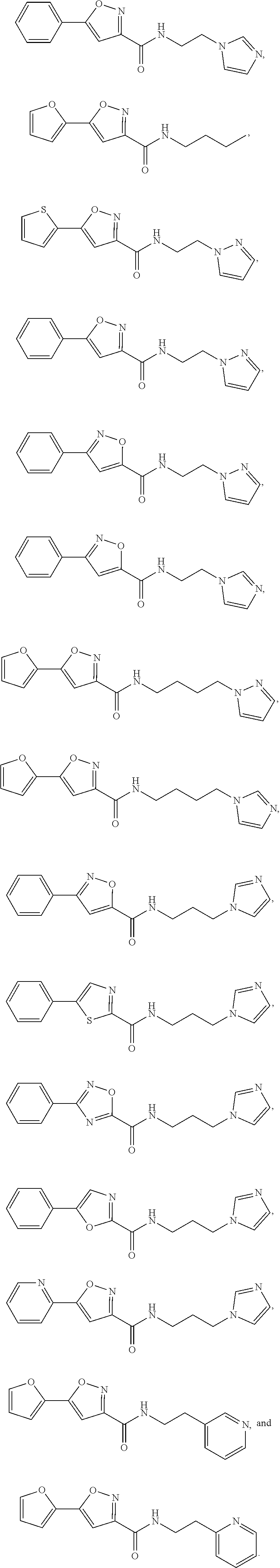

In yet another embodiment of any of the foregoing embodiments, the one or more agents that inhibit the mitogen-activated protein kinase interacting protein kinases 1 and 2 (Mnk1/2) is selected from CGP-57380, cercosporamide, BAY 1143269, tomivosertib, ETC-206, SLV-2436 and any combination thereof. In yet another or further embodiment, one or more agents that inhibit the PI3K pathway are selected from 3-methyladenine, LY294002, alpelisib, wortmannin, quercetin, hSMG-1 inhibitor 11j, zandelisib, alpelisib hydrochloride, idelalisib, buparlisib, copanlisib, IPI549, dactolisib, pictilisib, SAR405, duvelisib, fimepinostat, GDC-0077, PI-103, YM-20163, PF-04691502, Taselisib, omipalisib, samotolisib, isorhamnetin, ZATK474, parsaclisib, rigosertib, AZD8186, GSK2636771, disitertide, TG100-115, AS-605240, PI3K-IN-1, dactolisib tosylate, gedatolisib, TGX-221, umbralisib, AZD 6482, serabelisib, bimiralisib, apitolisib, alpha-linolenic acid, Vps34-PIK-III, PIK-93, Vps34-IN-1, CH5132799, leniolisib, voxtalisib, GSK1059615, sonolisib, PKI-402, PI4KIIIbeta-IN-9, HS-173, BGT226 maleate, pictilisib dimethane sulfonate, VS-5584, IC-87114, quercetin dihydrate, CNX-1351, SF2523, GDC-0326, seletalisib, acalisib, SAR-260301, ZAD-8835, GNE-317, AMG319, nemiralisib, IITZ-01, PI-103 hydrochloride, oroxin B, pilaralisib, AS-252424, cpanlisib dihydrochloride, AMG 511, disitertide TFA, PIK-90, tenalisib, esculetin, CGS 15943, GNE-477, PI-3065, A66, AZD3458, ginsenoside Rk1, sophocarpine, buparlisib hydrochloride, Vps34-IN-2, linperlisib, arnicolide D, KP372-1, CZC24832, PF-4989216, (R)-Duvelisib, PQR530, P11δ-IN-1, umbralisib hydrochloride, MTX-211, PI3K/mTOR Inhibitor-2, LX2343, PF-04979064, polygalasaponin F, glaucocalyxin A, NSC781406, MSC2360844, CAY10505, IPI-3063, TG 100713, BEBT-908, PI-828, brevianamide F, ETP-46321, PIK-294, SRX3207, sophocarpine monohydrate, AS-604850, desmethylglycitein, SKI V, WYE-687, NVP-QAV-572, GNE-493, CAL-130 hydrochloride, GS-9901, BGT226, IHMT-PI3K6-372, PI3Kα-IN-4, parsaclisib hydrochloride, PF-06843195, PI3K-IN-6, (S)-PI3Kα-IN-4, PI3K(gamma)-IN-8, BAY1082439, CYH33, PI3Kγ inhibitor 2, PI3K6 inhibitor 1, PARP/PI3K-IN-1, LAS191954, PI3K-IN-9, CHMFL-PI3KD-317, PI3K/HDAC-IN-1, MSC2360844 hemifumarate, PI3K-IN-2, PI3K/mTOR Inhibitor-1, PI3K6-IN-1, euscaphic acid, KU-0060648, AZD 6482, WYE-687 dihydrochloride, GSK2292767, (R)-Umbralisib, PIK-293, idelalisib D5, PIK-75, hirsutenone, quercetin D5, PIK-108, hSMG-1 inhibitor Ile, PI3K-IN-10, NVP-BAG956, PI3Kγ inhibitor 1, CAL-130, ON 146040, PI3kδ inhibitor 1, PI3Kα/mTOR-IN-1, and any combination thereof. In yet another or further embodiment, the growth factor is stem cell factor (SCF). In yet another or further embodiment, the B-Raf kinase inhibitor is selected from GDC-0879, PLX4032, GSK2118436, BMS-908662, LGX818, PLX3603, RAF265, RO5185426, vemurafenib, PLX8394, SB590885 and any combination thereof. In yet another or further embodiment, the B-Raf kinase inhibitor is GDC-0879. In yet another or further embodiment, the GMPs have the uniform morphology of being small, round-shaped, and/or non-adherent.

The disclosure provides a method for the expansion of a population of granulocyte/macrophage progenitor cells (GMPs) in a culture medium comprising: (i) a growth factor; (ii) a B-Raf kinase inhibitor; (iii) an agent that inhibits the mitogen-activated kinase interacting protein kinases 1 and 2 (Mnk1/2); (iv) an agent that inhibits the PI3K pathway; (v) optionally, one or more serum components; and (vi) a compound having the structure of Formula I:

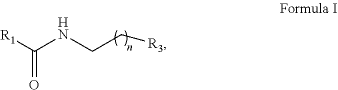

wherein, R¹ is selected from:

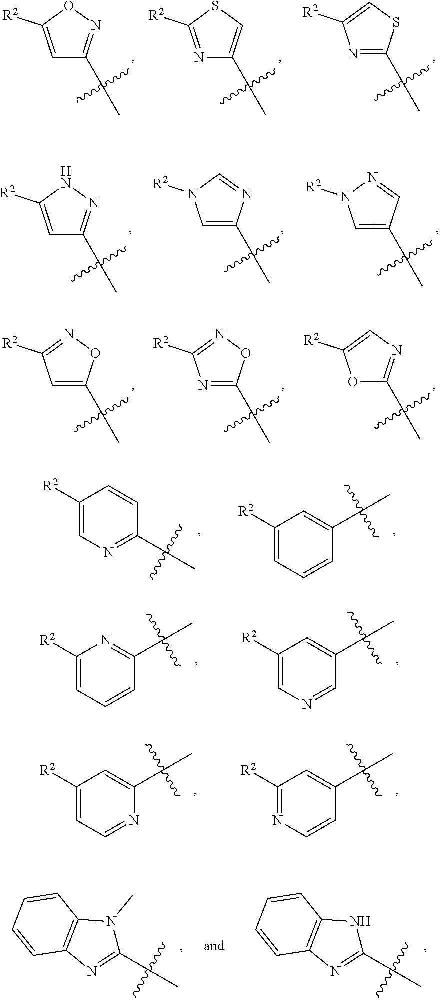

and R² is selected from:

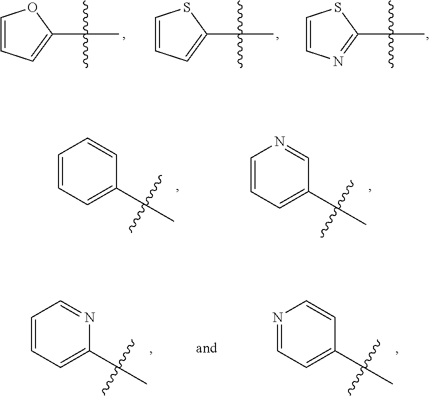

R³ is selected from:

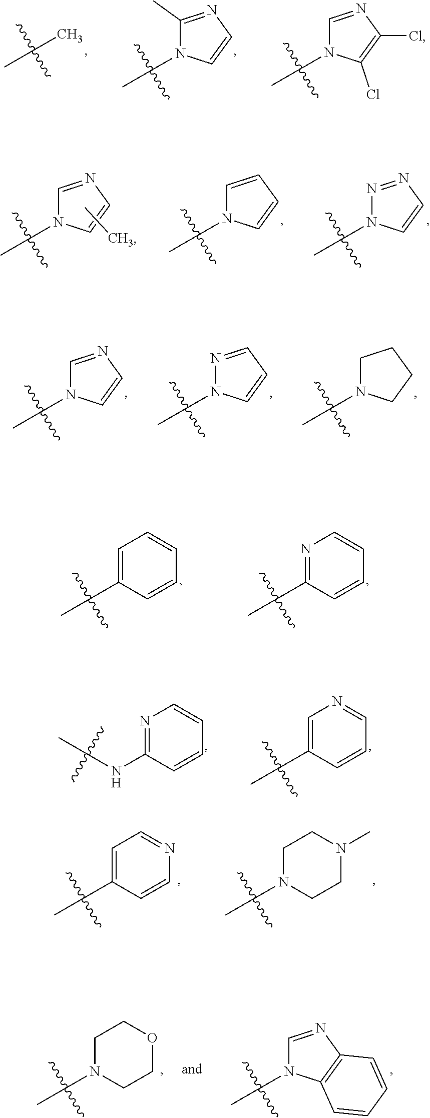

n is an integer selected from 0, 1, 2, 3, 4, and 5;
wherein the GMPs remain substantially morphologically unchanged after undergoing multiple cell passages and/or clonal expansion. In one embodiment, the GMPs are derived or obtained from stem cells. In a further embodiment, the stem cells are genetically engineered prior to or during culturing. In yet another or further embodiment, the stem cells are hematopoietic stem cells. In yet another or further embodiment, the hematopoietic stem cells are isolated from the bone marrow of a subject. In a further embodiment, the subject is a mammalian subject. In yet another or further embodiment, the subject is a human, a rat or a mouse. In yet another or further embodiment, the culture medium comprises DMEM/F12 and Neural Basal Medium. In yet another or further embodiment, the culture medium comprises DMEM/F12 and Neural Basal Medium in a ratio of about 5:1 to about 1:5. In yet another or further embodiment, the culture medium comprises DMEM/F12 and Neural Basal Medium in a ratio of about 1:1. In yet another or further embodiment, the culture medium comprises one or more supplements selected from insulin, transferrin, BSA fraction V, putrescine, sodium selenite, DL-α tocopherol, linolenic acid and/or linoleic acid. In yet another or further embodiment, the culture medium is supplemented with insulin, transferrin, BSA fraction V, putrescine, sodium selenite, DL-α tocopherol, and linolenic acid and/or linoleic acid. In a certain embodiment, the compound having the structure of Formula I is selected from:

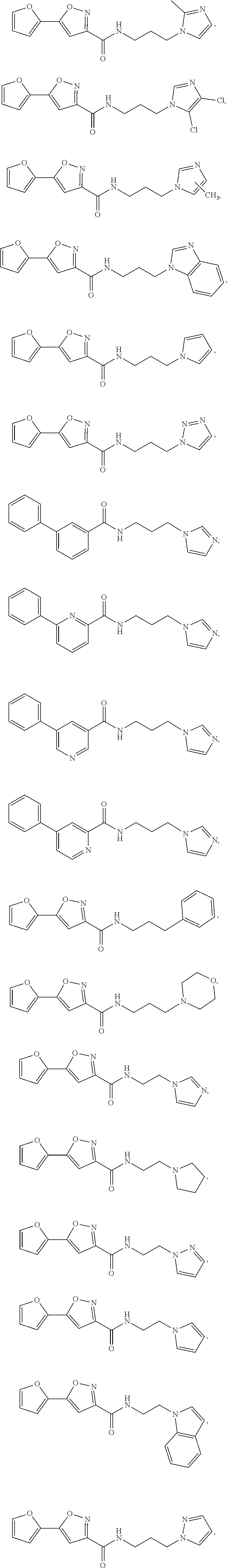

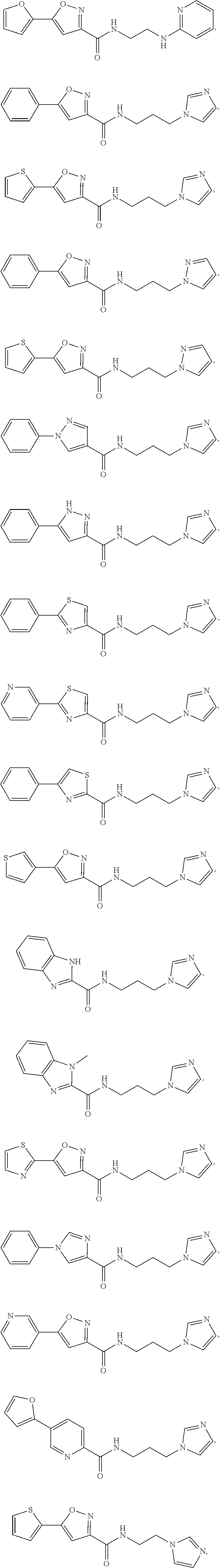

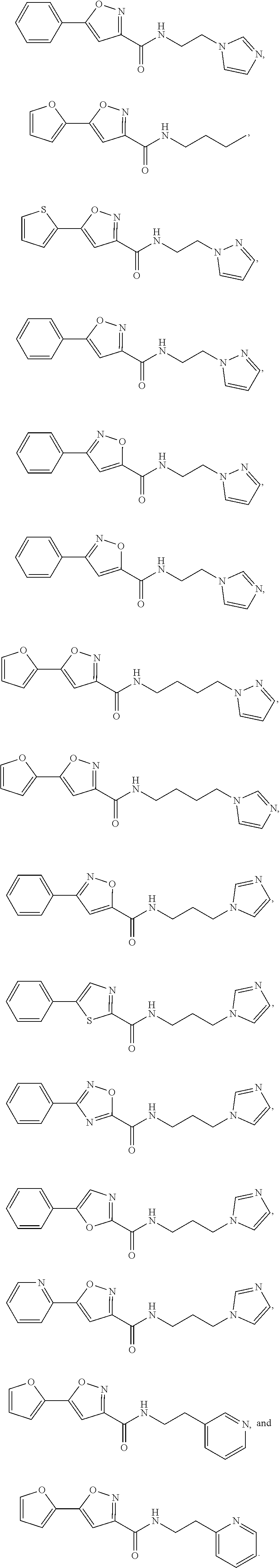

In yet another embodiment of any of the foregoing embodiments, the one or more agents that inhibit the mitogen-activated protein kinase interacting protein kinases 1 and 2 (Mnk1/2) is selected from CGP-57380, cercosporamide, BAY 1143269, tomivosertib, ETC-206, SLV-2436 and any combination thereof. In yet another or further embodiment, one or more agents that inhibit the PI3K pathway are selected from 3-methyladenine, LY294002, alpelisib, wortmannin, quercetin, hSMG-1 inhibitor 11j, zandelisib, alpelisib hydrochloride, idelalisib, buparlisib, copanlisib, IPI549, dactolisib, pictilisib, SAR405, duvelisib, fimepinostat, GDC-0077, PI-103, YM-20163, PF-04691502, Taselisib, omipalisib, samotolisib, isorhamnetin, ZATK474, parsaclisib, rigosertib, AZD8186, GSK2636771, disitertide, TG100-115, AS-605240, PI3K-IN-1, dactolisib tosylate, gedatolisib, TGX-221, umbralisib, AZD 6482, serabelisib, bimiralisib, apitolisib, alpha-linolenic acid, Vps34-PIK-III, PIK-93, Vps34-IN-1, CH5132799, leniolisib, voxtalisib, GSK1059615, sonolisib, PKI-402, PI4KIIIbeta-IN-9, HS-173, BGT226 maleate, pictilisib dimethane sulfonate, VS-5584, IC-87114, quercetin dihydrate, CNX-1351, SF2523, GDC-0326, seletalisib, acalisib, SAR-260301, ZAD-8835, GNE-317, AMG319, nemiralisib, IITZ-01, PI-103 hydrochloride, oroxin B, pilaralisib, AS-252424, cpanlisib dihydrochloride, AMG 511, disitertide TFA, PIK-90, tenalisib, esculetin, CGS 15943, GNE-477, PI-3065, A66, AZD3458, ginsenoside Rk1, sophocarpine, buparlisib hydrochloride, Vps34-IN-2, linperlisib, arnicolide D, KP372-1, CZC24832, PF-4989216, (R)-Duvelisib, PQR530, P11δ-IN-1, umbralisib hydrochloride, MTX-211, PI3K/mTOR Inhibitor-2, LX2343, PF-04979064, polygalasaponin F, glaucocalyxin A, NSC781406, MSC2360844, CAY10505, IPI-3063, TG 100713, BEBT-908, PI-828, brevianamide F, ETP-46321, PIK-294, SRX3207, sophocarpine monohydrate, AS-604850, desmethylglycitein, SKI V, WYE-687, NVP-QAV-572, GNE-493, CAL-130 hydrochloride, GS-9901, BGT226, IHMT-PI3K6-372, PI3Kα-IN-4, parsaclisib hydrochloride, PF-06843195, PI3K-IN-6, (S)-PI3Kα-IN-4, PI3K(gamma)-IN-8, BAY1082439, CYH33, PI3Kγ inhibitor 2, PI3K6 inhibitor 1, PARP/PI3K-IN-1, LAS191954, PI3K-IN-9, CHMFL-PI3KD-317, PI3K/HDAC-IN-1, MSC2360844 hemifumarate, PI3K-IN-2, PI3K/mTOR Inhibitor-1, PI3K6-IN-1, euscaphic acid, KU-0060648, AZD 6482, WYE-687 dihydrochloride, GSK2292767, (R)-Umbralisib, PIK-293, idelalisib D5, PIK-75, hirsutenone, quercetin D5, PIK-108, hSMG-1 inhibitor Ile, PI3K-IN-10, NVP-BAG956, PI3Kγ inhibitor 1, CAL-130, ON 146040, PI3kδ inhibitor 1, PI3Kα/mTOR-IN-1, and any combination thereof. In yet another or further embodiment, the growth factor is stem cell factor (SCF). In yet another or further embodiment, the B-Raf kinase inhibitor is selected from GDC-0879, PLX4032, GSK2118436, BMS-908662, LGX818, PLX3603, RAF265, RO5185426, vemurafenib, PLX8394, SB590885 and any combination thereof. In yet another or further embodiment, the B-Raf kinase inhibitor is GDC-0879. In yet another or further embodiment, the GMPs have the uniform morphology of being small, round-shaped, and/or non-adherent.

The disclosure also provides a method to genetically modify granulocyte/macrophage progenitor (GMPs) cells, comprising genetically engineering a modification into GMPs made by any of the foregoing methods using a gene editing system, homologous recombination, or site directed mutagenesis. In one embodiment, the gene editing system is a TALEN- or CRISPR-based system. In yet another or further embodiment, the genetically engineering modification comprises replacing or disrupting an existing gene (knockout), or altering a genetic locus to contain sequence information not found at the genetic locus (knock-in). In yet another or further embodiment, the genetically engineering modification of the GMPs comprises a knockout SIRPα and/or PI3Kγ gene. In another embodiment, of any for the foregoing, the method further comprises differentiating the GMPs into macrophages comprising culturing the GMPs with a macrophage differentiation medium comprising macrophage colony-stimulating factor (MCSF). In one embodiment, the macrophage differentiation medium comprises RPMI 1640, fetal bovine serum (FBS) and MCSF. In yet another or further embodiment, the differentiation medium comprises RPMI 1640, 10% FBS and 20 ng/mL of MCSF. In yet another or further embodiment of any of the foregoing, the method further comprises differentiating the GMPs into granulocytes comprising culturing the GMPs with a granulocyte differentiation medium comprising granulocyte colony-stimulating factor (GCSF). In yet another or further embodiment, the granulocyte differentiation medium comprises RPMI 1640, FBS and GCSF. In yet another or further embodiment, the granulocyte differentiation medium comprises RPMI 1640, 10% FBS and 20 ng/mL of GCSF.

The disclosure also provides a population of granulocyte/macrophage progenitor cells (GMPs) expanded by a method of the disclosure.

The disclosure also provides genetically modified granulocyte/macrophage progenitor cells (GMPs) prepared by a method of the disclosure.

The disclosure provides macrophages prepared by a method of the disclosure.

The disclosure provides granulocytes prepared by a method of the disclosure.

The disclosure also provides a pharmaceutical composition comprising an effective amount of the population of the GMPs, the genetically modified GMPs, macrophages, or granulocytes made by the methods disclosed herein, and a pharmaceutically acceptable carrier or excipient.

The disclosure also provides a method for treating or preventing a disease or condition in a subject in need comprising administering to said subject an effective amount of the population of the GMPs, the genetically modified GMPs, macrophages, or granulocytes made by the methods of the disclosure, and a pharmaceutically acceptable carrier or excipient.

The disclosure also provides for the use of an effective amount of the population of the GMPs, the genetically modified GMPs, macrophages, or granulocytes of the disclosure in the manufacture of a medicament for treating or preventing a disease or condition in a subject in need.

### DESCRIPTION OF DRAWINGS

FIG. 1 provides exemplary compounds of the disclosure that can be used in the methods of disclosure for the expansion of human granulocyte-macrophage progenitors and applications thereof.

FIG. 2 shows an overall strategy for the generation and expansion of GMPs from human iPSCs.

FIG. 3A-G shows the development of defined conditions for the long-term ex vivo expansion of mouse bone marrow-derived stem cells. (A) Schematic of the experimental design to identify growth factors and small molecules that can promote expansion of mouse bone marrow-derived stem cells. (B) Representative phase-contrast images of mouse bone marrow cells cultured in N2B27 supplemented with the indicated small molecule inhibitors for 7 days. (C) Representative phase-contrast images of mouse bone marrow cells cultured in N2B27 supplemented with SCF plus GDC0879 or SB590885 for 3 passages. (D) Cell growth curves. Bone marrow cells isolated from C57BL/6J mice were plated into 24-well plates at a density of 2×10⁵ cells/well and cultured in B7 medium supplemented with the indicated small molecules/growth factor. Cells were counted and passaged every 3 days. Data are represented as mean±SD from three independent experiments. (E) Bone marrow cells isolated from C57BL/6J mice were cultured in B7 medium supplemented with SCF/2i. Cells were passaged every 3 days. Representative phase-contrast pictures showing SCF/2i cells at different passages. (F) Cytogenetic analysis of SCF/2i cells using the GWT banding method. Bone marrow cells isolated from a female C57BL/6J mouse were expanded in SCF/2i for 8 passages before used for cytogenetic analysis. Out of 21 metaphases examined, all had a normal 40, XX karyotype. (G) Sequential images of individual bone marrow cells during expansion in B7 medium supplemented with SCF/2i. SCF/2i-expanded bone marrow cells were plated into 96-well plates at a density of 50 cells/well and cultured in SCF/2i. Sequential images were taken using the Keyence BZ-X710 microscopy every 24 hours. 15.7±4.0 colonies formed per well on day 4 (n=3×96). Data are represented as mean±SD from three independent experiments.

FIG. 4A-D provides for the characterization of SCF/2i-expanded cells. (A) Representative flow cytometry histograms showing the expression pattern of the indicated markers in SCF/2i-expanded cells (passage 3). Blue filled histograms, isotype control; red filled histograms, antibody staining. (B) t-SNE analysis of gene expression in SCF/2i-expanded cells (passage 3) and the indicated cell types freshly sorted from adult C57BL/6J mouse bone marrow. (C) Heatmap analysis showing the differential gene expression profiles among SCF/2i-expanded cells and the 5 primary cell types. (D) Violin plots from scRNA-seq showing the expression of lineage marker genes. Fcgr2b, Spi1 and Cebpa are markers for GMP; Ly6a, a marker for HSC; Ly6d, a marker for CLP; Epor, a marker for MEP.

FIG. 5A-G demonstrates SCF/2i GMPs can efficiently differentiate into macrophages and granulocytes in vitro. (A) Immunofluorescence and flow cytometry analysis of CD11b and F4/80 expression in cells differentiated from SCF/2i GMPs after treatment with M-CSF for 7 days. Flow cytometry data are represented as mean±SD from three independent experiments. (B) ELISA analysis of cytokine secretion in bone marrow (BM) and SCF/2i GMP-derived macrophages stimulated with 500 ng/ml LPS for 6 hours. Data are represented as mean±SD from three independent experiments. (C) Phagocytosis analysis of SCF/2i GMP-derived macrophages by incubating with FITC-labeled latex beads for 1 hour. Upper panel, a representative fluorescent image (green, FITC-labeled beads; blue, macrophage cell nuclei). Lower panel, flow cytometry analysis of SCF/2i GMP-derived macrophages incubated with (red) or without (blue) FITC-labeled latex beads. Flow cytometry data are represented as mean±SD from three independent experiments. (D) Time-lapse images of tdTomato-positive SCF/2i GMP-derived macrophages incubated with GFP labeled E. coli. The numbers on the images indicate the times in minutes. Arrows and arrowheads indicate the bacteria engulfed by macrophages. (E) Giemsa staining (upper panel) and flow cytometry analysis (lower panel) of SCF/2i GMPs treated with PBS or G-CSF for 3 days. Flow cytometry data are represented as mean±SD (n=5). (F) ELISA analysis of cytokine secretion and MPO activity measurement in the indicated cells stimulated with 500 ng/ml LPS for 6 hours (for ELISA assay) or 100 nM PMA for 2 hours (for MPO assay). Data are represented as mean±SD from three independent experiments. Ctrl, no treatment control; ns, not significant. (G) Single-cell colony forming assay of SCF/2i GMPs and GMPs freshly sorted from mouse bone marrow. Images showing representative colonies formed from individual SCF/2i GMPs 7 days after plating (M, macrophage only colony; G, granulocyte only colony; GM, granulocyte/macrophage colony). The histogram shows the percentage of each colony type. A total of 192×3 wells from three independent experiments were counted for each group. The average numbers of colonies formed from each experiment for SCF/2i and sorted GMP groups were 110±8.66 and 96±5.57, respectively. Data are represented as mean±SD.

FIG. 6A-E shows that SCF/2i GMPs differentiate into functional granulocytes and macrophages after transplantation. (A) Representative plots of flow cytometry analysis of peripheral blood samples collected from C57BL/6 mice transplanted with 1×10⁷ tdTomato-positive SCF/2i GMPs per mouse. G, granulocytes (CD11b⁺CD115⁻Ly6G⁺); M, macrophages (CD11b⁺CD115⁺). (B) Representative plots of flow cytometry analysis of peripheral blood samples collected from sublethally irradiated mice 4 days after transplantation of 1×10⁷ tdTomato-positive SCF/2i GMPs. Data are represented as mean±SD (n=3 mice). (C) Immuno-staining of liver tissue sections with anti-F4/80 and anti-tdToamto antibodies. Liver tissues were isolated from mice 7 days after transplantation with tdTomato-positive SCF/2i GMPs. Arrow indicates a F4/80 and tdTomato double positive cell. (D) Fluorescent images and flow cytometry analysis of peritoneal macrophages collected from C57BL/6 mice 4 days after transplantation of tdTomato-positive SCF/2i GMPs and intraperitoneal injection (IP) of PBS or 1 ml 2% bio-gel. Flow cytometry data are represented as mean±SD from three independent experiments. (E) Flow cytometry analysis of bone marrow and spleen cells collected from C57BL/6 mice one day after transplantation with 1×10⁷ tdTomato-positive SCF/2i GMPs per mouse. Data are represented as mean±SD (n=3 mice). M, macrophage; G, granulocyte.

FIG. 7A-F demonstrates that SCF/2i-expanded GMPs elicit therapeutic effects in a mouse model of bacterial infection. (A) Schematic diagram showing the timeline for irradiation, SCF/2i GMP transplantation, and bacteria inoculation. (B-D) CGD mice were injected with PBS or SCF/2i GMPs via tail vein at the indicated timepoints (A) and challenged with S. aureus. Mice were sacrificed 7 days after bacteria inoculation. The numbers of liver abscesses were counted (B), the weights of spleen masses were measured (C), and the survival rates of mice were calculated (D) (n=10 mice for each group). (E) Survival rates of CGD mice inoculated with B. Cepacia and transplanted with SCF/2i GMPs (n=10 mice for each group). (F) Collection and inoculation of blood samples from CGD mice inoculated with B. Cepacia and transplanted with SCF/2i GMPs or PBS. 50 μl of blood sample was collected from each CGD mouse 7 days after inoculation with B. Cepacia and was inoculated onto a 10 cm agar plate and incubated at 37° C. for 16 hours. Left, representative images of blood culture. Right, quantification of colony-forming units (CFU) for bacteria.

FIG. 8A-H shows the expansion, differentiation, and genetic engineering of human GMPs. (A) Growth curves of human GMPs. Human GMPs were FACS sorted from cord blood and cultured in the designated conditions. Cells were passaged every 3 days and replated into 12-well plates at a density of 1×10⁵ cells/well. Data are represented as mean±SD from three independent experiments. (B) Structure of TN-2-30. (C) Representative phase-contrast images of human GMPs cultured in modified SCF/2i. P, passage number. (D) Sorted human GMPs were expanded in modified SCF/2i and induced to differentiate towards granulocytes by treatment with 30 ng/ml human G-CSF for 10 days. Differentiated cells were analyzed by Giemsa staining (upper panel) and flow cytometry (lower panel). Flow cytometry data are represented as mean±SD from three independent experiments. (E) Relative MPO activities of human GMPs and human GMP-derived granulocytes stimulated with or without PMA for 2 hours. Differentiated cells generated in (D) were plated into 96-well plates at a density of 2×10⁴ cells/well and stimulated with or without PMA for 2 hours, after which MPO activities in the supernatants were measured. Data are represented as mean±SD from three independent experiments. (F) Immunofluorescence and flow cytometry analysis of the expression of human macrophage markers CD68 and CD14 in cells differentiated from ex vivo expanded human GMPs. Human GMPs were induced to differentiate towards macrophages by culturing in DMEM/10% FBS supplemented with 20 ng/ml human M-CSF for 10 days. Flow cytometry data are represented as mean±SD from three independent experiments. (G) Phagocytosis analysis of human GMP-derived macrophages by incubating with GFP-labeled E. coli for one hour. Representative phase-contrast and fluorescent images showing the GFP-labeled bacteria engulfed by macrophages and a representative plot of flow cytometry analysis of human GMP-derived macrophages incubated with (red) or without (blue) GFP-labeled bacteria. Flow cytometry data are represented as mean±SD from three independent experiments. (H) Differentiated cells generated in (D) and (F) were plated into 96-well plates at a density of 2×10⁴ cells/well and stimulated with or without 500 ng/ml LPS for 6 hours, after which cytokine secretion in the supernatants was measured by ELISA. Data are represented as mean±SD from three independent experiments.

### DETAILED DESCRIPTION

As used herein and in the appended claims, the singular forms “a,” “an,” and “the” include plural referents unless the context clearly dictates otherwise. Thus, for example, reference to “a cell” includes a plurality of cells and reference to “the granulocyte-macrophage progenitor” includes reference to one or more granulocyte-macrophage progenitors and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this invention is not limited to the particular methodology, protocols, and reagents, etc., described herein and as such may vary. The terminology used herein is for the purpose of describing particular embodiments only and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%. The term “about” includes an amount or ratio that would be expected to be within experimental error.

As used herein, the term “administering,” refers to the placement an agent (e.g., an engineered GMP or macrophage or granulocyte derived therefrom) as disclosed herein into a subject by a method or route which results in at least partial localization of the agents at a desired site.

“Autologous cells” as used herein refers to cells derived from the same individual as to whom the cells are later to be re-administered.

A “B-Raf kinase inhibitor” refers to a substance, e.g., a compound or molecule, that blocks or reduces an activity of a protein called B-Raf kinase, or reduces an amount of B-Raf kinase. B-Raf is a kinase enzyme that helps control cell growth and signaling. It may be found in a mutated (changed) form in some types of cancer, including melanoma and colorectal cancer. Some B-Raf kinase inhibitors are used to treat cancer. Examples of B-Raf kinase inhibitor include, but are not limited to, GDC-0879, PLX4032, GSK2118436, BMS-908662, LGX818, PLX3603, RAF265, R05185426, vemurafenib, PLX8394, and SB590885. In a particular embodiment, a method disclosed herein comprises use of the B-Raf kinase inhibitor GDC-0879.

The term “effective amount” or “therapeutically effective amount” as used herein refers to the amount of a composition comprising GMPs (or macrophages or granulocytes derived therefrom) that decrease at least one or more symptom of the disease or disorder, and relates to a sufficient amount of the composition to provide the desired effect. The phrase “therapeutically effective amount” as used herein means a sufficient amount of the composition to treat a disorder, at a reasonable benefit/risk ratio applicable to any medical treatment.

In certain instances, a therapeutically or prophylactically significant reduction in a symptom includes, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more increased, enhanced, or elevated in a measured parameter as compared to a control or non-treated subject or the state of the subject prior to administering the cellular compositions described herein. In some instances, a therapeutically or prophylactically significant reduction in a symptom includes, e.g., at least about 40%, at least about 50%, at least about 60%, at least about 70%, at least about 80%, at least about 90% or more reduced, suppressed, inhibited in a measured parameter as compared to a control or non-treated subject or the state of the subject prior to administering the cellular compositions described herein. Measured or measurable parameters include clinically detectable markers of disease, for example, elevated or depressed levels of a biological marker. The exact amount required will vary depending on factors such as the type of disease being treated, gender, age, and weight of the subject.

“Granulocyte colony-stimulating factor” or “GCSF” (also known as colony-stimulating factor 3 (CSF 3)), is a glycoprotein that stimulates the bone marrow to produce granulocytes and stem cells. The gene sequence, protein sequence and orthologs across various species are known in the art (see, e.g., NCBI Reference Sequence: NP_000750.1, which is incorporated herein by reference).

A “growth factor” refers to a substance, e.g., a compound or molecule, that is effective to promote cell growth, cell proliferation, or cell differentiation, e.g., stem cells, and which, unless added to the culture medium as a supplement, is not otherwise a component of the basal medium. Growth factors include, but are not limited to, stem cell factor (SCF), basic fibroblast growth factor (bFGF), acidic fibroblast growth factor (aFGF), epidermal growth factor (EGF), insulin-like growth factor-I (IGF-I), insulin-like growth factor-II (IGF-II), platelet-derived growth factor-AB (PDGF), and vascular endothelial cell growth factor (VEGF), activin-A, Wnt and bone morphogenic proteins (BMPs), insulin, cytokines, chemokines, morphogens, neutralizing antibodies, other proteins, and small molecules. Exogenous growth factors may also be added to a medium according to the disclosure to assist in the maintenance of cultures of GMPs in a substantially undifferentiated state. Such factors and their effective concentrations can be identified as described elsewhere herein or using techniques known to those of skill in the art of culturing cells. In a particular embodiment, the GMPs are cultured in a culture medium which comprises SCF.

The term “isolated” as used herein refers to molecules, biologicals, cells or cellular materials being substantially free from other materials for which it is normally associated. In one aspect, the term “isolated” refers to nucleic acid, such as DNA or RNA, or protein or polypeptide (e.g., an antibody or derivative thereof), or cell or cellular organelle, separated from other DNAs or RNAs, or proteins or polypeptides, or cells or cellular organelles, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both, cultured and engineered cells or tissues.

As used herein a “long term culture” or “long term expansion” refers to the propagation of cells under controlled conditions such that the cells expand in number and/or maintain substantial viability and substantially similar morphology. In some embodiments the term refers to the time period of culture while maintaining a desired morphology and cell number (e.g., for about two months or longer) or may be associated with the number of passages (e.g., media changes) of at least 10 media passages. In other embodiments the term refers to the increase in number over a period of time (e.g., an increase by at least one million times in a about a two-month period). In some embodiments, the long-term cultures are cultured for more than 4 months, more than 6 months or more than 1 year. In other embodiments, the long-term cultures are passaged for more than 15 passages, more than 18 passages or more than 20 passages.

“Macrophage colony-stimulating factor” or “MCSF” (also known as colony-stimulating factor 1 (CSF 1)), is involved in the proliferation, differentiation, and survival of monocytes, macrophages, and bone marrow progenitor cells. The gene sequence, protein sequence and orthologs across various species are known in the art (see, e.g., NCBI Reference Sequence: NP 000748.4, which is incorporated herein by reference).

“Polynucleotide” as used herein includes but is not limited to DNA, RNA, cDNA (complementary DNA), mRNA (messenger RNA), rRNA (ribosomal RNA), shRNA (small hairpin RNA), snRNA (small nuclear RNA), snoRNA (short nucleolar RNA), miRNA (microRNA), genomic DNA, synthetic DNA, synthetic RNA, and/or tRNA.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A typical alignment program is BLAST, using default parameters. In particular, typical programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

It is to be inferred without explicit recitation and unless otherwise intended, that when the present disclosure relates to a polypeptide, protein, polynucleotide, antibody or fragment thereof, an equivalent or a biologically equivalent of such is intended within the scope of this disclosure. As used herein, the term “biological equivalent thereof” is intended to be synonymous with “equivalent thereof” when referring to a reference protein, antibody or fragment thereof, polypeptide or nucleic acid, intends those having minimal homology while still maintaining desired structure or functionality. Unless specifically recited herein, it is contemplated that any of the above also includes equivalents thereof. For example, an equivalent intends at least about 70% homology or identity, or at least 80% homology or identity and alternatively, or at least about 85%, or alternatively at least about 90%, or alternatively at least about 95%, or alternatively at least 98% percent homology or identity and exhibits substantially equivalent biological activity to the reference protein, polypeptide, antibody or fragment thereof or nucleic acid. Alternatively, when referring to polynucleotides, an equivalent thereof is a polynucleotide that hybridizes under stringent conditions to the reference polynucleotide or its complement. Alternatively, when referring to polypeptides or proteins, an equivalent thereof is a expressed polypeptide or protein from a polynucleotide that hybridizes under stringent conditions to the polynucleotide or its complement that encodes the reference polypeptide or protein.

“Stem Cell Factor” or “SCF” (also known as KIT-ligand, KL, or steel factor) is a cytokine that binds to the c-KIT receptor (CD117). SCF can exist both as a transmembrane protein and a soluble protein. This cytokine plays an important role in hematopoiesis (formation of blood cells), spermatogenesis, and melanogenesis. The gene sequence, protein sequence and orthologs across various species are known in the art (see, e.g., NCBI Reference Sequence NP_000890.1, which is incorporated herein by reference).

As used herein a “substantially uniform population” refers to a population of cells in which at least 80% of the cells are of the indicated type, preferably at least 90%, 95%, or even 98% or more.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder, such as cancer. Treatment is generally “effective” if one or more symptoms or clinical markers are reduced. Additionally or alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment). In some embodiments, treatment of cancer includes decreasing tumor volume, decreasing the number of cancer cells, inhibiting cancer metastases, increasing life expectancy, decreasing cancer cell proliferation, decreasing cancer cell survival, or amelioration of various physiological symptoms associated with the cancerous condition.

A “Wnt activator” refers to compound or molecule that induces Wnt signaling pathways. The Wnt signaling pathways are a group of signal transduction pathways which begin with proteins that pass signals into a cell through cell surface receptors. Three Wnt signaling pathways have been characterized: the canonical Wnt pathway, the noncanonical planar cell polarity pathway, and the noncanonical Wnt/calcium pathway. All three pathways are activated by the binding of a Wnt-protein ligand to a Frizzled family receptor, which passes the biological signal to the Disheveled protein inside the cell. Wnt comprises a diverse family of secreted lipid-modified signaling glycoproteins that are 350-400 amino acids in length. The type of lipid modification that occurs on these proteins is palmitoylation of cysteines in a conserved pattern of 23-24 cysteine residues. Palmitoylation is necessary because it initiates targeting of the Wnt protein to the plasma membrane for secretion and it allows the Wnt protein to bind its receptor due to the covalent attachment of fatty acids. Wnt proteins also undergo glycosylation, which attaches a carbohydrate in order to ensure proper secretion. In Wnt signaling, these proteins act as ligands to activate the different Wnt pathways via paracrine and autocrine routes. These proteins are highly conserved across species. They can be found in mice, humans, Xenopus, zebrafish, Drosophila and many others. Examples of Wnt activators includes, but are not limited to, SKL 2001, BML-284, WAY 262611, CAS 853220-52-7, and QS11. In a particular embodiment, methods disclosed herein comprise use of a compound of the disclosure which has Wnt activator activity.

Granulocytes, macrophages, and dendritic cells originate from a common progenitor in the bone marrow, the granulocyte-macrophage progenitor (GMP). Despite the immense therapeutic potential of innate immune cells, their application in the clinic has been greatly limited because of the current inability to effectively expand and genetically modify these cells or their progenitors GMPs. Provided herein are methods for the long-term expansion and/or maintenance of mouse and human GMPs. These conditions can likely also be used for the expansion of GMPs derived from other species. Ex vivo expanded GMPs can efficiently differentiate into mature and functional granulocytes, macrophages, and dendritic cells both in vitro and in vivo. These ex vivo expanded GMPs can also be genetically modified. The methods disclosed herein for the production of GMPs, and the GMPs produced therefrom, have great utility because: (1) long-term expansion of human GMPs provide unlimited homogenous cell populations for both basic research and clinical applications; (2) long-term expansion of human GMPs allows for the study of regulation of an immune response by modifying GMP genes, and their expression thereof; and (3) ex vivo expanded human GMPs can be used for clinical applications, including transplantation. For example, ex vivo expanded human GMPs can readily be used to treat neutropenia. Moreover, the disclosure also provides for the genetic modification of human GMPs (e.g., knockout SIRPα and/or PI3Kγ gene; overexpression of angiotensin converting enzyme), which can be further induced to differentiate into macrophages and dendritic cells. These engineered macrophages and dendritic cells are expected to have enhanced antitumor effects and can be used clinically to treat cancer, either as monotherapy or combination therapy with other immunological agents, such as anti-PD-1/PD-L1 antibodies and chimeric antigen receptor T (CAR-T) cells.

Macrophages display divergent phenotypes that were originally classified as M1 or M2 polarity. M1 polarized macrophages display the capacity to present antigen, produce IL-12, IL-23, interferon gamma (IFNγ), and reactive oxygen species (ROS). M1 macrophages are more effective at antitumor and skewing T cell responses toward a T helper type 1 (Th1) or cell mediated immune response. In contrast, M2 macrophages produce IL-10 and TGF-β and participate in tissue remodeling, have immunosuppressive qualities, and promote Th2 or antibody mediated immune responses. Tumor-associated macrophages (TAMs) constitute a major component of the tumor microenvironment. These cells are predominant M2 phenotype macrophages which promote tumor immunosuppression. Recent studies support their contribution to the suppression of T cell function, which is not abolished by the use of Immune checkpoint blockage. Macrophages have therefore become an attractive therapeutic target to combat cancer. Despite the huge therapeutic potential of macrophages, their application in clinic has been greatly limited because currently there is no effective method to expand and genetically modify macrophages or their progenitors GMPs. Long-term expansion of human GMPs allows for genetic modification to make these cells more therapeutically applicable.

It was found that inhibition of two protein kinases, the mitogen-activated protein kinase kinase (MEK) and glycogen synthase kinase 3 (GSK3), allows long-term self-renewal of mouse and rat embryonic stem cells (ESCs). Based on this finding, it was postulated that many, if not all, types of stem cells can be maintained during long-term in vitro culture by inhibiting signaling pathways responsible for initiating differentiation. In an attempt to identify inhibitors that can promote self-renewal of stem/progenitor cells of the hematopoietic system, bone marrow cells isolated from adult C57BL/6 mice were plated into 96-well plates in serum free N2B27 medium and then screened with small molecule libraries. A B-Raf kinase inhibitor (GDC-0879; “GDC”) was identified that could significantly promote the formation of colonies containing uniform bright, small and round-shaped cells. After passaging, however, these cells gradually attached and differentiated. Accordingly, another round screening was performed. The second screening identified a Wnt activator (SKL2001; “SKL”) that acted synergistically with the B-Raf kinase inhibitor, GDC-0879, to promote the expansion of the uniform round-shaped cells. The combination of GDC and SKL, however, was not found to be sufficient for the long-term expansion of the cells. A third screen was performed. In this screen, a panel of growth factors were identified as possibly being important for the long-term expansion of the cells. After further experimentation with mouse cells, it was found that methods which utilized stem cell factor (SCF) in combination with the B-Raf kinase inhibitor (GDC-0879) and the Wnt activator (SKL2001) allowed for the production of a uniform mouse GMP cell population of bright, small and round-shaped cells which could further undergo long term cell expansion. While this formulation was found to be very effective with mouse GMPs, it had limited effectiveness on human GMP cells. Accordingly, there was a need to identify compounds which could effectively expand human GMPs.

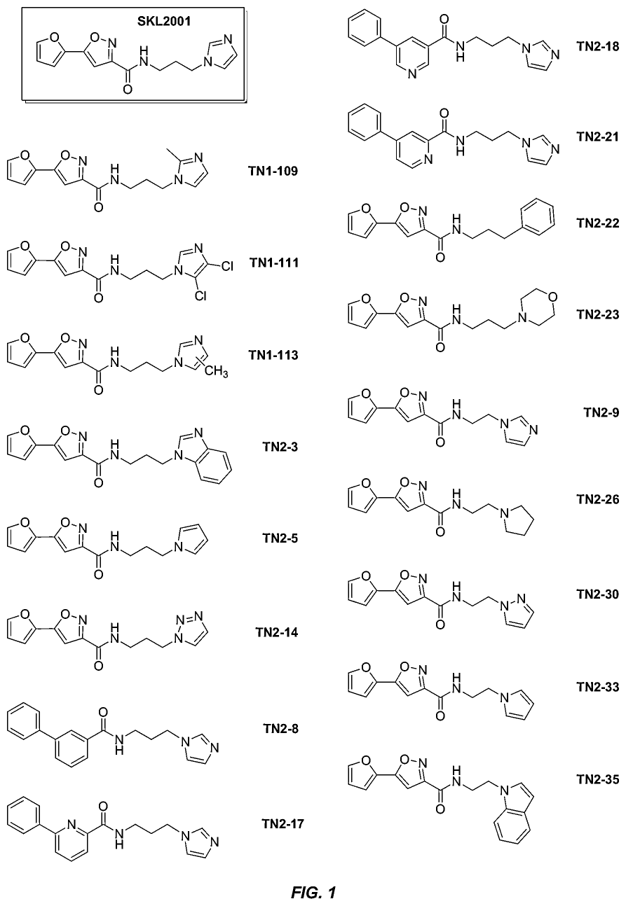

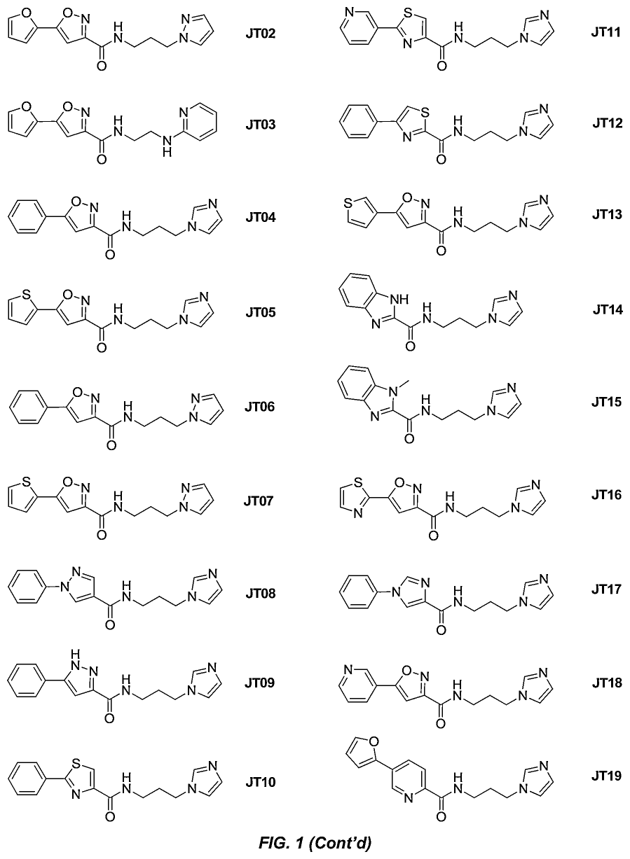

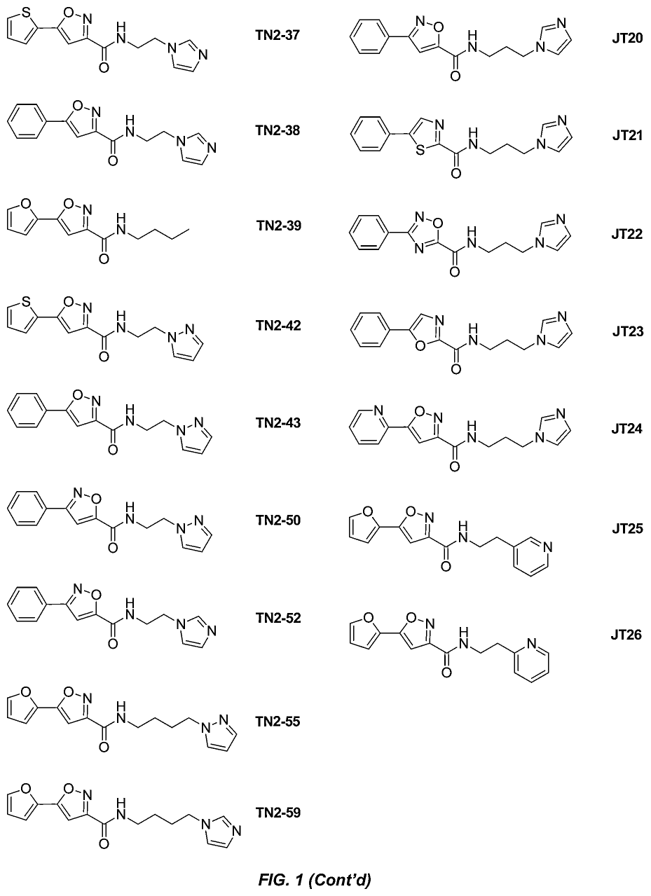

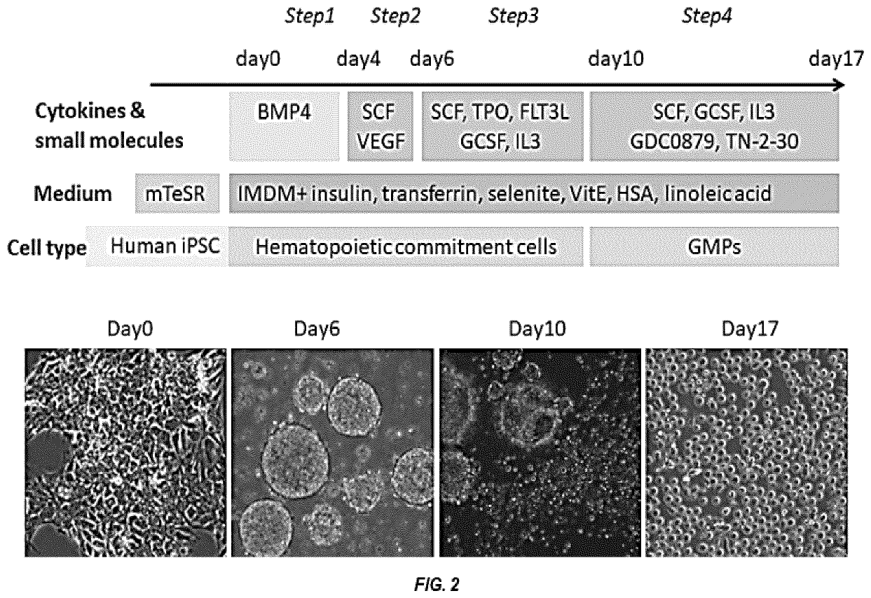

The disclosure provides new compounds which can be used in the methods of the disclosure for the expansion of human GMP cells (see, Formula I and FIG. 1). Thus, in various embodiments presented herein, methods of the disclosure utilize the compounds disclosed herein with other agents to promote the long-term maintenance and/or expansion of GMPs. In yet a further embodiment, the methods of the disclosure utilize the compounds of the disclosure to promote the long-term expansion and/or maintenance of human and other GMPs.

In a particular embodiment, the disclosure provides a method for the long-term expansion and/or maintenance of a uniform cell population of granulocyte/macrophage progenitor cells (GMPs) that remain morphologically unchanged (e.g., substantially maintain the morphological characteristics such as shape, size and the like) after undergoing multiple cell passages and clonal expansion. In a further embodiment, a method disclosed herein comprises culturing GMPs in a culture medium which includes a combination of at least two, at least three, at least four factors and agents including, but not limited to, a growth factor (e.g., SCF), a B-Raf kinase inhibitor (e.g., GDC-0879), a compound of the disclosure (see, e.g., formula I and FIG. 1), an agent that inhibits Mnk1/2, an agent that inhibits the PI3K pathway, and optionally, one or more serum components.

In one embodiment, the GMPs disclosed herein are derived or produced from stem cells. Stem cells can include embryonic stem cells, induced pluripotent stem cells, non-embryonic (adult) stem cells, and cord blood stem cells. Stem cell types that can be cultured using the media of the disclosure include stem cells derived from any mammalian species including humans, mice, rats, monkeys, and apes (see, e.g., Okita et al., Nature 448:313-318, July 2007; and Takahashi et al., Cell 131(5):861-872; which are incorporated herein by reference).

Stem cells are cells capable of differentiation into other cell types, including those having a particular, specialized function (e.g., tissue specific cells, parenchymal cells and progenitors thereof). Progenitor cells (i.e., “multipotent”) are cells that can give rise to different terminally differentiated cell types, and cells that are capable of giving rise to various progenitor cells. Cells that give rise to some or many, but not all, of the cell types of an organism are often termed “pluripotent” stem cells, which are able to differentiate into any cell type in the body of a mature organism, although without reprogramming they are unable to de-differentiate into the cells from which they were derived. As will be appreciated, “multipotent” stem/progenitor cells (e.g., granulocyte/macrophage progenitor cells (GMPs)) have a narrower differentiation potential than do pluripotent stem cells. Prior to derivation into GMPs, the stem cells disclosed herein can be genetically modified by use of any number of genetic engineering techniques, e.g., such as gene therapy, gene editing systems, homologous recombination, etc. Such modified stem cells may provide for enhanced therapies (e.g., see Nowakowski et al., Acta Neurobiol Exp (Wars) 73(1):1-18 (2013)).

In a particular embodiment, the GMPs of the disclosure are derived from induced pluripotent stem cells (iPSs, or iPSCs). iPSCs are pluripotent stem cells obtained from non-pluripotent cells by selective gene expression (of endogenous genes) or by transfection with a heterologous gene. Induced pluripotent stem cells are described by Shinya Yamanaka's team at Kyoto University, Japan. Yamanaka had identified genes that are particularly active in embryonic stem cells, and used retroviruses to transfect mouse fibroblasts with a selection of those genes. Eventually, four key pluripotency genes essential for the production of pluripotent stem cells were isolated; Oct-3/4, SOX2, c-Myc, and Klf4. Cells were isolated by antibiotic selection for Fbx15⁺ cells. The same group published a study along with two other independent research groups from Harvard, MIT, and the University of California, Los Angeles, showing successful reprogramming of mouse fibroblasts into iPS and even producing a viable chimera. The process of inducing pluripotent stem cells is well characterized in the art. Moreover, on going research has reduced the number of factors necessary to induced pluripotency.

In some embodiments, the GMPs disclosed herein are derived from embryonic stem cells (ESCs). ESCs are stem cells derived from the undifferentiated inner mass cells of a human embryo. Embryonic stem cells are pluripotent, meaning they are able to grow and differentiate into all derivatives of the three primary germ layers: ectoderm, endoderm and mesoderm. Pluripotency distinguishes embryonic stem cells from adult stem cells found in adults; while embryonic stem cells can generate all cell types in the body, adult stem cells are multipotent and can produce only a limited number of cell types. Additionally, under defined conditions, embryonic stem cells are capable of propagating themselves indefinitely. This allows embryonic stem cells to be employed as useful tools for both research and regenerative medicine, because they can produce limitless numbers of themselves for continued research or clinical use.

In some embodiments, the GMPs disclosed herein are derived from cord blood stem cells. Umbilical cord blood is the blood left over in the placenta and in the umbilical cord after the birth of the baby. The cord blood is composed of all the elements found in whole blood. It contains red blood cells, white blood cells, plasma, platelets and is also rich in hematopoietic stem cells. Hematopoietic stem cells can be isolated from cord blood using any number of isolation methods taught in the art, including those taught in Chularojmontri et al., J Med Assoc Thai 92(3):S88-94 (2009). Moreover, commercial kits are available for isolation of CD34⁺ cells (i.e., hematopoietic stem cells) from human umbilical cord blood. These kits are available from multiple vendors, including STEMCELL Technologies, Thermo Fisher Scientific, Zen-Bio, etc.

In some embodiments, the GMPs disclosed herein are derived from non-embryonic stem cells. The non-embryonic stem cell can renew and can differentiate to yield some or all of the major specialized cell types of a tissue or organ. The primary roles of non-embryonic stem cells in a living organism are to maintain and repair the tissue in which they are found. Scientists also use the term somatic stem cell instead of non-embryonic stem cell, where somatic refers to cells of the body (not the germ cells, sperm or eggs). Non-embryonic stem cells have been identified in many organs and tissues, including brain, bone marrow, peripheral blood, blood vessels, skeletal muscle, skin, teeth, heart, gut, liver, ovarian epithelium, and testis. They are thought to reside in a specific area of each tissue (called a “stem cell niche”). In a living animal, non-embryonic stem cells are available to divide for a long period, when needed, and can give rise to mature cell types that have characteristic shapes and specialized structures and functions of a particular tissue.

In certain embodiments, the GMPs disclosed herein are derived from hematopoietic stem cells (HSCs). HSCs can be isolated from umbilical cord blood and bone marrow. In some instances, the HSCs can be isolated using isolation protocols that are known in the art, which typically use CD34⁺ as a cell selection marker for the isolation of HSCs (e.g., see Lagasse et al., Nat Med. 6:1229-1234 (2000), which is incorporated herein by reference).

In the methods disclosed herein, the GMPs can be grown and expanded in a culture medium which includes a combination of at least two, at least three, at least four factors and agents including, but not limited to, a growth factor (e.g., SCF), a B-Raf kinase inhibitor (e.g., GDC-0879), a compound of the disclosure (see, e.g., formula I and FIG. 1), an agent that inhibits Mnk1/2, an agent that inhibits the PI3K pathway, and optionally, one or more serum components. In some instances, the culture medium comprises a compound of the disclosure (see, e.g., formula I and FIG. 1). In some instances, the culture medium comprises a compound of the disclosure (see, e.g., formula I and FIG. 1), and further comprises at least one of a growth factor (e.g., SCF), a B-Raf kinase inhibitor (e.g., GDC-0879), an agent that inhibits Mnk1/2, an agent that inhibits the PI3K pathway. In some instances, the culture medium comprises a growth factor (e.g., SCF), a B-Raf kinase inhibitor (e.g., GDC-0879), a compound of the disclosure (see, e.g., formula I and FIG. 1), an agent that inhibits Mnk1/2, and an agent that inhibits the PI3K pathway. Optionally, the culture medium comprises one or more serum components. In some instances, the culture medium comprises a modified basal medium that is supplemented with various other biological agents. A basal medium refers to a solution of amino acids, vitamins, salts, and nutrients that is effective to support the growth of cells in culture, although normally these compounds will not support cell growth unless supplemented with additional compounds. The nutrients include a carbon source (e.g., a sugar such as glucose) that can be metabolized by the cells, as well as other compounds necessary for the cell's survival. These are compounds that the cells themselves cannot synthesize, due to the absence of one or more of the gene(s) that encode the protein(s) necessary to synthesize the compound (e.g., essential amino acids) or, with respect to compounds which the cells can synthesize, because of their particular developmental state the gene(s) encoding the necessary biosynthetic proteins are not being expressed as sufficient levels. Various basal media are known in the art of mammalian cell culture, such as Dulbecco's Modified Eagle Media (DMEM), RPMI 1640, Knockout-DMEM (KO-DMEM), and DMEM/F12, although any base medium that can be supplemented with agents which supports the growth of stem cells in a substantially undifferentiated state can be employed. The disclosure further demonstrates, that a culture medium that comprises a ratio of one of the basal medias exemplified above (e.g., DMEM/F12) with a neural basal medium (or alternatively other basal medium such as IMDM and/or StemSpan™ SFEMII) unexpectedly provided for improved growth of the GMPs. In particular, a ratio of about 5:1 to about 1:5 of one of the basal medias exemplified above (e.g., DMEM/F12) to a neural basal medium can be used to culture the GMPs. In a further embodiment, the culture medium for growing GMPs comprises about 1:1 of DMEM/F12 to a neural basal media.

The culture medium disclosed herein for growing GMPs may be supplemented with one or more additional agents, including, but not limited to insulin, transferrin, BSA fraction V, putrescine, sodium selenite, DL-α tocopherol, and linolenic acid and/or linoleic acid. In a certain embodiment, the culture medium disclosed herein for growing GMPs is supplemented with insulin, transferrin, BSA fraction V, putrescine, sodium selenite, DL-α tocopherol, and linolenic acid and/or linoleic acid.

As will be appreciated, it is desirable to replace spent culture medium with fresh culture medium either continually, or at periodic intervals, typically every 1 to 3 days. One advantage of using fresh medium is the ability to adjust conditions so that the cells expand more uniformly and rapidly than they do when cultured on feeder cells according to conventional techniques, or in conditioned medium.

Populations of GMPs can be obtained that are 4-, 10-, 20-, 50-, 100-, 1000-, or more fold expanded when compared to the initial or a previous starting cell population. Under suitable conditions, cells in the expanded population will be 50%, 70%, or more in the undifferentiated state, as compared to the GMPs used to initiate the culture. The degree of expansion per passage can be calculated by dividing the approximate number of cells harvested at the end of the culture by the approximate number of cells originally seeded into the culture. Where geometry of the growth environment is limiting or for other reasons, the cells may optionally be passaged into a similar growth environment for further expansion. The total expansion is the product of all the expansions in each of the passages. Of course, it is not necessary to retain all the expanded cells on each passage. For example, if the cells expand two-fold in each culture, but only about 50% of the cells are retained on each passage, then approximately the same number of cells will be carried forward. But after four cultures, the cells are said to have undergone an expansion of 16-fold. Cells may be stored by cryogenic freezing techniques known in the art.

As indicated in more detail herein, the GMPs can be grown and expanded in a culture medium which includes a combination of at least two, at least three, at least four factors and agents including, but not limited to, a growth factor (e.g., SCF), a B-Raf kinase inhibitor (e.g., GDC-0879), a compound of the disclosure, an agent that inhibits Mnk1/2, an agent that inhibits the PI3K pathway, and optionally, one or more serum components.

The disclosure provides for methods and/or compositions for cell culture or expansion, which comprise one or more compounds having the structure of Formula I:

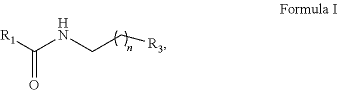

wherein,
R¹ is selected from:

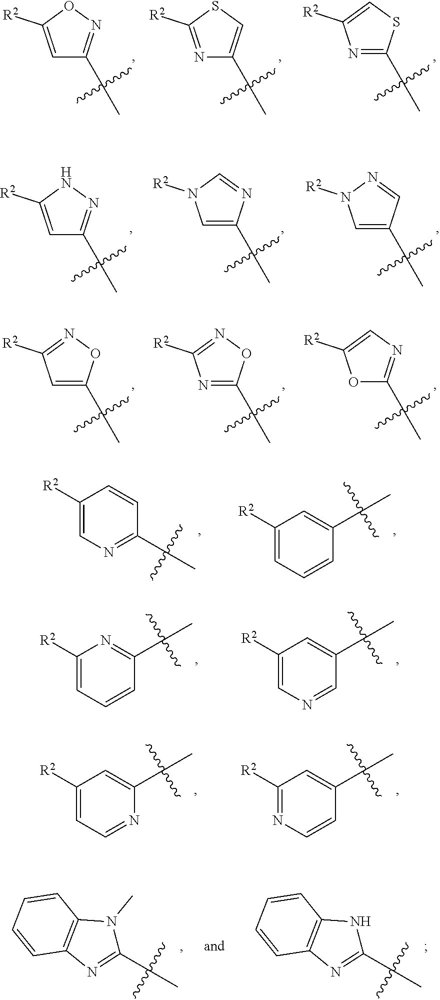

R² is selected from:

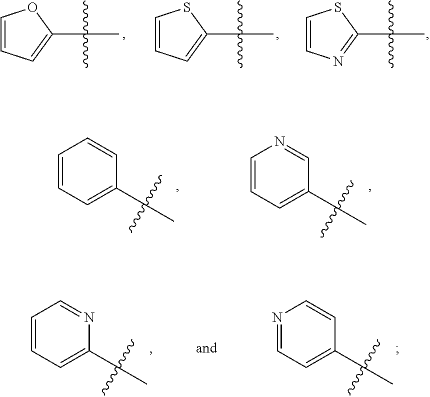

R³ is selected from:

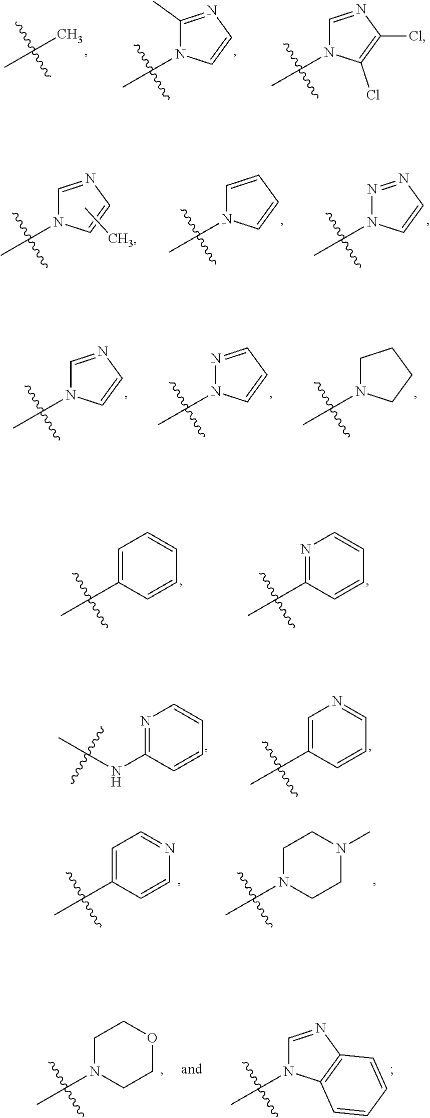

n is an integer selected from 0, 1, 2, 3, 4, and 5. In a further embodiment, a compound having the structure of Formula I is not

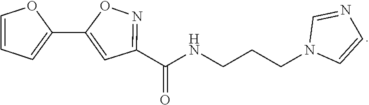 [SMILES: O=C(NCCCn1ccnc1)c1cc(-c2ccco2)on1]

In some embodiments, the disclosure provides for methods and/or compositions for cell culture or expansion, which comprise one or more compounds having the structure of:

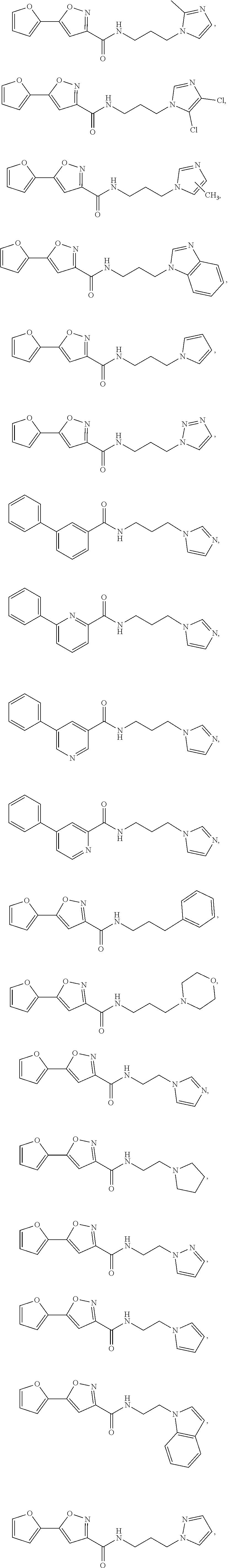

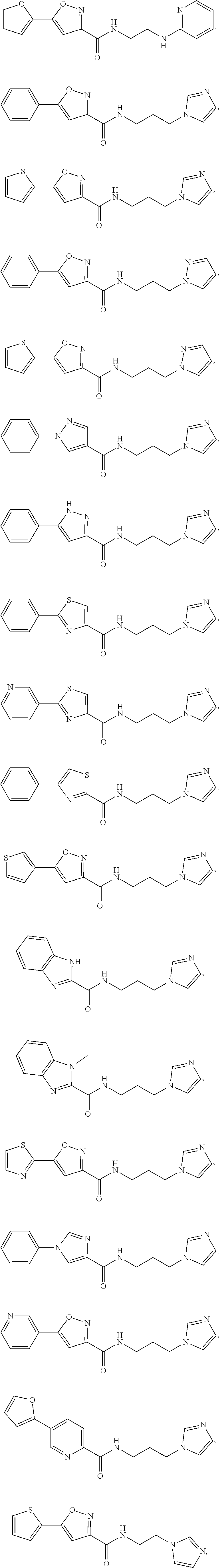

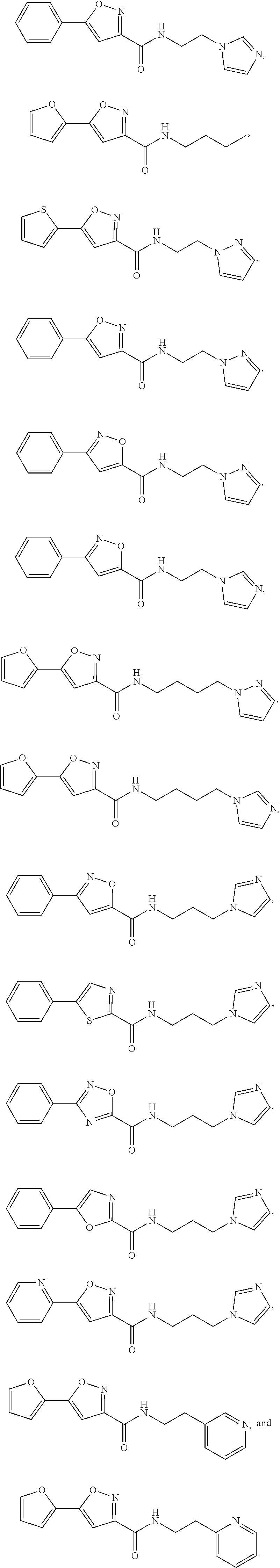

The disclosure further provides methods to genetically modify the GMPs disclosed herein using genetic engineering techniques. In particular it was shown herein that the GMPs of the disclosure are susceptible to genetic modification techniques, thereby allowing for the use of the GMPs in basic scientific research and clinical therapeutic applications. Thus, expanded and genetically modified GMPs can be readily translated into broad clinical applications. Accordingly, the disclosure further provides methods to genetically modify GMPs disclosed herein. Such methods, can include the step of genetically engineering modifications into GMPs by using a gene editing system, homologous recombination, or site directed mutagenesis. Particular examples of gene editing systems include zing finger nucleases, TALEN and CRISPR.

In a certain embodiment, the CRISPR system is a type II CRISPR system and the Cas enzyme is Cas9, which catalyzes DNA cleavage. Enzymatic action by Cas9 derived from Streptococcus pyogenes or any closely related Cas9 generates double stranded breaks at target site sequences which hybridize to 20 nucleotides of the guide sequence and that have a protospacer-adjacent motif (PAM) sequence (examples include NGG/NRG or a PAM that can be determined as described herein) following the 20 nucleotides of the target sequence. CRISPR activity through Cas9 for site-specific DNA recognition and cleavage is defined by the guide sequence, the tracr sequence that hybridizes in part to the guide sequence and the PAM sequence. More aspects of the CRISPR system are described in Karginov and Hannon, The CRISPR system: small RNA-guided defence in bacteria and archaea, Mole Cell 2010, January 15; 37(1): 7.

The type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). In this system, targeted DNA double-strand break (DSB) is generated in four sequential steps. First, two non-coding RNAs, the pre-crRNA array and tracrRNA, are transcribed from the CRISPR locus. Second, tracrRNA hybridizes to the direct repeats of pre-crRNA, which is then processed into mature crRNAs containing individual spacer sequences. Third, the mature crRNA:tracrRNA complex directs Cas9 to target sequences comprising the protospacer and the corresponding PAM via heteroduplex formation between the spacer region of the crRNA and the protospacer DNA. Finally, Cas9 mediates cleavage of target sequence of PAM to create a DSB within the protospacer. In a certain embodiment, the RNA polymerase Ill-based U6 promoter is to drive the expression of tracrRNA.

Typically, in the context of an endogenous CRISPR system, formation of a CRISPR complex (comprising a guide sequence hybridized to a target sequence and complexed with one or more Cas proteins) results in cleavage of one or both strands in or near (e.g., within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, one or more vectors driving expression of one or more elements of a CRISPR system are introduced into a host cell (e.g., a GMP or stem cell) such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a CRISPR expression vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. In some embodiments, a vector comprises an insertion site upstream of a tracr mate sequence, and optionally downstream of a regulatory element operably linked to the tracr mate sequence, such that following insertion of a guide sequence into the insertion site and upon expression the guide sequence directs sequence-specific binding of a CRISPR complex to a target sequence in a eukaryotic cell (e.g., a GMP or stem cell). In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these. When multiple different guide sequences are used, a single expression construct may be used to target CRISPR activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to a cell.

In some embodiments, a vector comprises a regulatory element operably linked to an enzyme-coding sequence encoding a CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In some embodiments, the unmodified CRISPR enzyme has DNA cleavage activity, such as Cas9. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9a nickase include, without limitation, H840A, N854A, and N863A. As a further example, two or more catalytic domains of Cas9 (RuvC I, RuvC II, and RuvC III or the HNH domain) may be mutated to produce a mutated Cas9 substantially lacking all DNA cleavage activity. In some embodiments, a D10A mutation is combined with one or more of H840A, N854A, or N863A mutations to produce a Cas9 enzyme substantially lacking all DNA cleavage activity. In some embodiments, a CRISPR enzyme is considered to substantially lack all DNA cleavage activity when the DNA cleavage activity of the mutated enzyme is less than about 25%, 10%, 5%, 1%, 0.1%, 0.01%, or lower with respect to its non-mutated form. Where the enzyme is not SpCas9, mutations may be made at any or all residues corresponding to positions 10, 762, 840, 854, 863 and/or 986 of SpCas9 (which may be ascertained for instance by standard sequence comparison tools. In particular, any or all of the following mutations are preferred in SpCas9: D10A, E762A, H840A, N854A, N863A and/or D986A; as well as conservative substitution for any of the replacement amino acids is also envisaged. The same (or conservative substitutions of these mutations) at corresponding positions in other Cas9s are also indicated.

Indicated orthologs are also described herein. A Cas enzyme may be identified Cas9 as this can refer to the general class of enzymes that share homology to the biggest nuclease with multiple nuclease domains from the type II CRISPR system. Most preferably, the Cas9 enzyme is from, or is derived from, spCas9 or saCas9. By derived, it is meant that the derived enzyme is largely based, in the sense of having a high degree of sequence homology with, a wildtype enzyme, but that it has been mutated (modified) in some way as described herein.

It will be appreciated that the terms Cas and CRISPR enzyme are generally used herein interchangeably, unless otherwise apparent. As mentioned above, many of the residue numberings used herein refer to the Cas9 enzyme from the type II CRISPR locus in Streptococcus pyogenes. However, it will be appreciated that this disclosure includes many more Cas9s from other species of microbes, such as SpCas9, SaCa9, St1Cas9 and so forth.

The gene editing systems (e.g., zing finger nucleases, CRISPR and TALEN) can be used to genetically engineer modifications into the GMP or stem cells, such as replacing or disrupting an existing gene found in the GMP or stem cell (knockout). As shown in the Examples presented herein, the GMPs of the disclosure are particular susceptible to knockout mutations. Moreover, it is expected that additional knockouts could be easily created from the GMPs of the disclosure such as SIRPα gene knockouts and/or a PI3Kγ gene knockouts. Alternatively, the same editing systems (e.g., CRISPR and TALEN) can be used to alter a genetic locus to contain sequence information not found at the genetic locus (a knock-in mutation). Such modifications, can be used to create GMP's that have “gained a function.” Such modified GMPs are particular useful for mimicking a disease state, e.g., by expressing biomolecules associated with a disease or disorder.

The disclosure further provides for the differentiation of the GMPs into myeloid and lymphoid lineages of blood cells, such as monocytes, macrophages, granulocytes, neutrophils, basophils, eosinophils, erythrocytes, megakaryocytes to platelets, T cells, B cells, and natural killer cells. In a particular embodiment, a method disclosed herein further comprises differentiating the GMPs of the disclosure into macrophages by culturing the GMPs with a macrophage differentiation medium comprising MCSF. In yet a further embodiment, the macrophage differentiation medium comprises RPMI 1640, 10% FBS and 20 ng/mL of MCSF. In an alternate embodiment, a method disclosed herein further comprises differentiating the GMPs of the disclosure into granulocytes comprising: culturing the GMPs with a granulocyte differentiation medium comprising GCSF. In yet a further embodiment, the granulocyte differentiation medium comprises RPMI 1640, 10% FBS and 20 ng/mL of GCSF.

The following examples are intended to illustrate but not limit the disclosure. While they are typical of those that might be used, other procedures known to those skilled in the art may alternatively be used.

### EXAMPLES

### Synthetic Methods and Characterizations of SKL2001 Analogs.

Compounds described were prepared according to the method depicted in Schemes 1 to 6

General Procedure 1: Amide Formation from Carboxylic Acid with Propanephosphonic Acid (T3P)

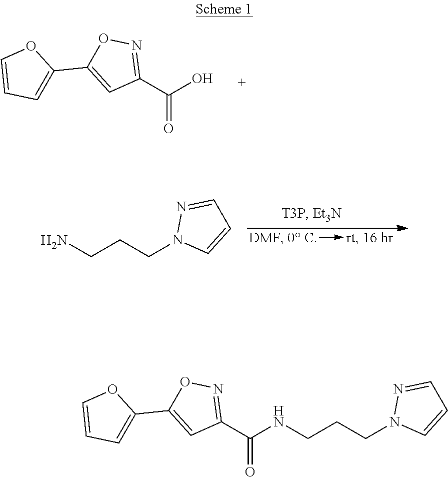

### N-(3-(1H-pyrazol-1-yl)propyl)-5-(furan-2-yl)isoxazole-3-carboxamide

To a mixture of 5-furan-2-yl-isoxazole-3-carboxylic acid (100 mg, 0.56 mmol), 3-(1H-pyrazol-1-yl)propan-1-amine (73 PL, 0.61 mmol) and triethylamine (233 μL, 1.67 mmol) in DMF (1.5 mL), propanephosphonic acid anhydride (T3P, 50% w/w in DMF) (393 μL, 0.61 mmol) was slowly added at 0° C. The solution was warmed to room temperature and stirred for 16 hours. Brine was added, then the aqueous layer was extracted with EtOAc three times. The combined organic layer was dried over NaSO₄, filtered, and evaporated. The crude material was purified via flash column chromatography (100% EtOAc) to provide the title compound (137 mg, 86% yield) as a white solid.

¹H NMR (400 MHz, Chloroform-d) δ 7.56 (dd, J=1.8, 0.8 Hz, 1H), 7.52 (dd, J=1.9, 0.7 Hz, 1H), 7.42 (dd, J=2.3, 0.7 Hz, 1H), 7.30 (t, J=5.7 Hz, 1H), 6.93 (dd, J=3.5, 0.7 Hz, 1H), 6.83 (s, 1H), 6.54 (dd, J=3.5, 1.8 Hz, 1H), 6.24 (t, J=2.1 Hz, 1H), 4.25 (t, J=6.5 Hz, 2H), 3.45 (q, J=6.4 Hz, 2H), 2.17 (p, J=6.5 Hz, 3H). MS (ESI):287.11[M+H]+.

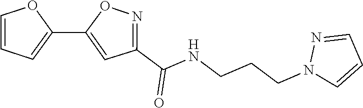 [SMILES: O=C(NCCCn1cccn1)c1cc(-c2ccco2)on1]

### N-(3-(1H-pyrazol-1-yl)propyl)-5-(thiophen-2-yl)isoxazole-3-carboxamide

General Procedure 1 (Scheme 1): Flash chromatography (Hexane/EtOAc=30/70). 88% yield as a yellow solid.

¹H NMR (400 MHz, Chloroform-d) δ 7.53 (m, 2H), 7.47 (dd, J=5.0, 1.2 Hz, 1H), 7.43 (d, J=2.1 Hz, 1H), 7.31 (t, J=6.0 Hz, 1H), 7.13 (dd, J=5.0, 3.7 Hz, 1H), 6.79 (s, 1H), 6.24 (t, J=2.1 Hz, 1H), 4.25 (t, J=6.5 Hz, 2H), 3.45 (q, J=6.4 Hz, 2H), 2.17 (p, J=6.5 Hz, 2H). MS (ESI):303.09[M+H]+.

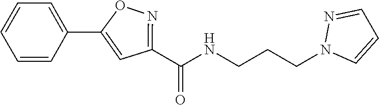 [SMILES: O=C(NCCCn1cccn1)c1cc(-c2ccccc2)on1]

### N-(3-(1H-pyrazol-1-yl)propyl)-5-phenylisoxazole-3-carboxamide

General Procedure 1 (Scheme 1): Flash chromatography (Hexane/EtOAc=30/70). 84% yield as a pale-yellow solid.

¹H NMR (400 MHz, Chloroform-d) δ 7.79 (m, 2H), 7.54 (dd, J=1.9, 0.7 Hz, 1H), 7.47 (m, 4H), 7.30 (t, J=5.5 Hz, 1H), 6.95 (s, 1H), 6.26 (t, J=2.1 Hz, 1H), 4.27 (t, J=6.5 Hz, 2H), 3.47 (q, J=6.4 Hz, 2H), 2.19 (p, J=6.5 Hz, 2H). MS (ESI):297.13 [M+H]⁺.

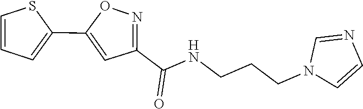 [SMILES: O=C(NCCCn1ccnc1)c1cc(-c2cccs2)on1]

### N-(3-(1H-imidazol-1-yl)propyl)-5-(thiophen-2-yl)isoxazole-3-carboxamide

General Procedure 1 (Scheme 1): Flash chromatography (EtOAc/MeOH=90/10). 68% yield as a yellow solid.

¹H NMR (400 MHz, Chloroform-d) δ 7.53 (s, 1H), 7.52 (dd, J=2.6, 1.1 Hz, 1H), 7.48 (dd, J=5.0, 1.1 Hz, 1H), 7.31 (t, J=6.2 Hz, 1H), 7.13 (dd, J=5.0, 3.7 Hz, 1H), 7.06 (s, 1H), 6.96 (s, 1H), 6.81 (s, 1H), 4.05 (t, J=7.0 Hz, 2H), 3.47 (q, J=6.6 Hz, 2H), 2.12 (p, J=6.9 Hz, 2H). MS (ESI):303.09[M+H]⁺.

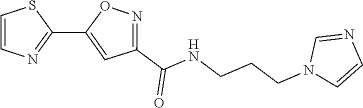 [SMILES: O=C(NCCCn1ccnc1)c1cc(-c2nccs2)on1]

### N-(3-(1H-imidazol-1-yl)propyl)-5-(thiazol-2-yl)isoxazole-3-carboxamide

General Procedure 1 (Scheme 1): Flash chromatography: (EtOAc/MeOH=90/10). 52% yield as a pale-yellow solid.

¹H NMR (400 MHz, Chloroform-d) δ 8.02 (d, J=3.1 Hz, 1H), 7.57 (d, J=3.2 Hz, 1H), 7.56 (s, 1H), 7.25 (s, 1H), 7.10 (m, 2H), 6.99 (s, 1H), 4.07 (t, J=7.0 Hz, 2H), 3.49 (q, J=6.6 Hz, 2H), 2.15 (p, J=6.9 Hz, 2H).

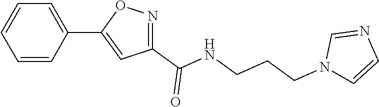 [SMILES: O=C(NCCCn1ccnc1)c1cc(-c2ccccc2)on1]

### N-(3-(1H-imidazol-1-yl)propyl)-5-phenylisoxazole-3-carboxamide

General Procedure 1 (Scheme 1): Flash chromatography (EtOAc/MeOH=90/10). 89% yield as a pale-yellow solid.

¹H NMR (400 MHz, Chloroform-d) δ 7.76 (m, 2H), 7.52 (s, 1H), 7.45 (m, 3H), 7.06 (s, 1H), 6.96 (s, 1H), 6.95 (s, 1H), 4.04 (t, J=7.0 Hz, 2H), 3.47 (q, J=6.6 Hz, 2H), 2.12 (p, J=6.9 Hz, 2H). MS (ESI):297.13 [M+H]⁺.

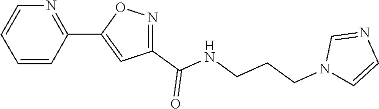 [SMILES: O=C(NCCCn1ccnc1)c1cc(-c2ccccn2)on1]

### N-(3-(1H-imidazol-1-yl)propyl)-5-(pyridin-2-yl)isoxazole-3-carboxamide

General Procedure 1 (Scheme 1): Flash chromatography: (EtOAc/MeOH=90/10). 43% yield as a white solid.

¹H NMR (400 MHz, Chloroform-d) δ 8.72 (ddd, J=4.8, 1.7, 1.0 Hz, 1H), 7.86 (m, 2H), 7.62 (s, 1H), 7.37 (ddd, J=7.3, 4.8, 1.5 Hz, 1H), 7.30 (s, 1H), 7.14 (m, 2H), 7.03 (s, 1H), 4.07 (t, J=7.0 Hz, 2H), 3.49 (q, J=6.6 Hz, 2H), 2.15 (p, J=6.9 Hz, 2H).

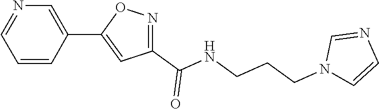 [SMILES: O=C(NCCCn1ccnc1)c1cc(-c2cccnc2)on1]

### N-(3-(1H-imidazol-1-yl)propyl)-5-(pyridin-3-yl)isoxazole-3-carboxamide

General Procedure 1 (Scheme 1): Flash chromatography: (EtOAc/MeOH=80/20). 41% yield as a yellow solid.

¹H NMR (400 MHz, Chloroform-d) δ 9.06 (dd, J=2.3, 0.9 Hz, 1H), 8.72 (dd, J=4.9, 1.7 Hz, 1H), 8.09 (ddd, J=8.0, 2.3, 1.6 Hz, 1H), 7.63 (s, 1H), 7.45 (ddd, J=8.0, 4.9, 0.9 Hz, 1H), 7.10 (s, 1H), 7.07 (s, 1H), 7.05 (t, J=5.4 Hz, 1H), 6.99 (s, 1H), 4.08 (t, J=7.0 Hz, 2H), 3.50 (q, J=6.6 Hz, 2H), 2.15 (p, J=6.9 Hz, 2H).

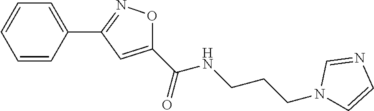 [SMILES: O=C(NCCCn1ccnc1)c1cc(-c2ccccc2)no1]

### N-(3-(1H-imidazol-1-yl)propyl)-3-phenylisoxazole-5-carboxamide

General Procedure 1 (Scheme 1): Flash chromatography: (EtOAc/MeOH=90/10). 62% yield as a yellow solid.

¹H NMR (400 MHz, Chloroform-d) δ 7.80 (m, 2H), 7.61 (s, 1H), 7.47 (m, 3H), 7.43 (t, J=5.7 Hz, 1H), 7.23 (s, 1H), 7.11 (s, 1H), 7.01 (s, 1H), 4.08 (t, J=6.9 Hz, 2H), 3.49 (q, J=6.6 Hz, 2H), 2.16 (p, J=6.8 Hz, 2H). MS (ESI):297.13[M+H]⁺.

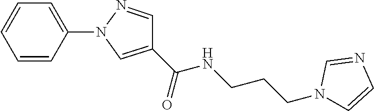 [SMILES: O=C(NCCCn1ccnc1)c1cnn(-c2ccccc2)c1]

### N-(3-(1H-imidazol-1-yl)propyl)-1-phenyl-1H-pyrazole-4-carboxamide

General Procedure 1 (Scheme 1): Flash chromatography: (EtOAc/MeOH=90/10). 67% yield as a white solid.

¹H NMR (600 MHz, Chloroform-d) δ 8.48 (s, 1H), 7.97 (s, 1H), 7.84 (s, 1H), 7.67 (m, 2H), 7.44 (t, J=7.7 Hz, 2H), 7.32 (t, J=7.4 Hz, 1H), 7.11 (s, 1H), 7.01 (s, 1H), 6.55 (s, 1H), 4.11 (t, J=6.7 Hz, 2H), 3.46 (q, J=6.3 Hz, 2H), 2.15 (p, J=6.6 Hz, 2H).

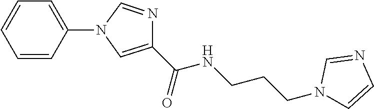 [SMILES: O=C(NCCCn1ccnc1)c1cn(-c2ccccc2)cn1]

### N-(3-(1H-imidazol-1-yl)propyl)-1-phenyl-1H-imidazole-4-carboxamide

General Procedure 1 (Scheme 1): Flash chromatography: (EtOAc/MeOH=90/10). 32% yield as colorless oil.

¹H NMR (400 MHz, Chloroform-d) δ 7.92 (d, J=1.4 Hz, 1H), 7.77 (d, J=1.4 Hz, 1H), 7.56 (s, 1H), 7.51 (m, 2H), 7.41 (m, 3H), 7.07 (s, 1H), 6.99 (s, 1H), 4.06 (t, J=7.1 Hz, 2H), 3.48 (td, J=6.4, 3.0 Hz, 2H), 2.11 (p, J=6.8 Hz, 2H).

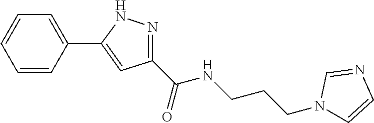 [SMILES: O=C(NCCCn1ccnc1)c1cc(-c2ccccc2)[nH]n1]

### )N-(3-(1H-imidazol-1-yl)propyl)-5-phenyl-1H-pyrazole-3-carboxamide

General Procedure 1 (Scheme 1): Flash chromatography: (EtOAc/MeOH=90/10). 81% yield as a white solid.

¹H NMR (600 MHz, Chloroform-d) δ 7.88 (s, 1H), 7.65 (d, J=7.6 Hz, 2H), 7.42 (t, J=7.6 Hz, 2H), 7.36 (m, 2H), 7.11 (s, 1H), 7.08 (s, 1H), 7.00 (s, 1H), 4.10 (t, J=6.8 Hz, 2H), 3.49 (s, 1H), 3.46 (q, J=6.3 Hz, 2H), 2.14 (p, J=6.7 Hz, 2H).

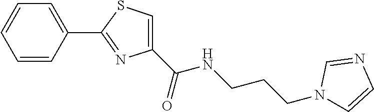 [SMILES: O=C(NCCCn1ccnc1)c1csc(-c2ccccc2)n1]

### N-(3-(1H-imidazol-1-yl)propyl)-2-phenylthiazole-4-carboxamide

General Procedure 1 (Scheme 1): Flash chromatography: (EtOAc/MeOH=90/10). 79% yield as a pale-yellow solid.

¹H NMR (600 MHz, Chloroform-d) δ 8.11 (s, 1H), 7.96 (m, 2H), 7.69 (s, 1H), 7.56 (t, J=6.4 Hz, 1H), 7.48 (m, 3H), 7.12 (s, 1H), 7.04 (s, 1H), 4.10 (t, J=7.0 Hz, 2H), 3.52 (q, J=6.6 Hz, 2H), 2.17 (p, J=6.8 Hz, 2H).

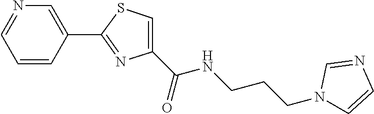 [SMILES: O=C(NCCCn1ccnc1)c1csc(-c2cccnc2)n1]

### N-(3-(1H-imidazol-1-yl)propyl)-2-(pyridin-3-yl)thiazole-4-carboxamide

General Procedure 1 (Scheme 1): Flash chromatography: (EtOAc/MeOH=90/10). 71% yield as a pale-yellow solid.

¹H NMR (600 MHz, Chloroform-d) δ 9.17 (d, J=2.3 Hz, 1H), 8.69 (dd, J=4.9, 1.6 Hz, 1H), 8.21 (dt, J=8.0, 2.1 Hz, 1H), 8.16 (s, 1H), 7.56 (m, 2H), 7.41 (dd, J=7.7, 4.9 Hz, 1H), 7.07 (s, 1H), 7.00 (s, 1H), 4.07 (t, J=7.0 Hz, 2H), 3.51 (q, J=6.6 Hz, 2H), 2.15 (p, J=6.9 Hz, 2H).

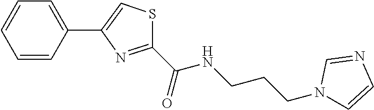 [SMILES: O=C(NCCCn1ccnc1)c1nc(-c2ccccc2)cs1]

### N-(3-(1H-imidazol-1-yl)propyl)-4-phenylthiazole-2-carboxamide

General Procedure 1 (Scheme 1): Flash chromatography: (EtOAc/MeOH=90/10). 54% yield as colorless oil.

¹H NMR (600 MHz, Chloroform-d) δ 7.87 (m, 2H), 7.70 (s, 1H), 7.61 (m, 2H), 7.43 (t, J=7.4 Hz, 2H), 7.36 (tt, J=7.3, 1.3 Hz, 1H), 7.08 (s, 1H), 6.99 (s, 1H), 4.05 (t, J=7.0 Hz, 2H), 3.49 (q, J=6.6 Hz, 2H), 2.14 (p, J=6.9 Hz, 2H).

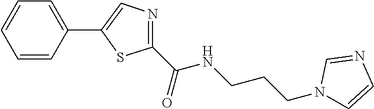 [SMILES: O=C(NCCCn1ccnc1)c1ncc(-c2ccccc2)s1]

### N-(3-(1H-imidazol-1-yl)propyl)-5-phenylthiazole-2-carboxamide

General Procedure 1 (Scheme 1): Flash chromatography: (EtOAc/MeOH=90/10). 47% yield as a pale-yellow solid.

¹H NMR (400 MHz, Chloroform-d) δ δ 7.99 (s, 1H), 7.80 (s, 1H), 7.61 (m, 2H), 7.44 (m, 3H), 7.36 (t, J=6.5 Hz, 1H), 7.18 (s, 1H), 7.11 (s, 1H), 4.12 (t, J=6.9 Hz, 2H), 3.51 (q, J=6.5 Hz, 2H), 2.17 (p, J=6.8 Hz, 2H). MS (ESI):313.11[M+H]⁺.

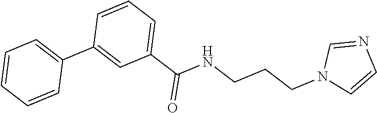 [SMILES: O=C(NCCCn1ccnc1)c1cccc(-c2ccccc2)c1]

### N-(3-(1H-imidazol-1-yl) propyl)-[1,1′-biphenyl]-3-carboxamide

General Procedure 1 (Scheme 1): Flash chromatography: (EtOAc/MeOH=90/10). 51% yield as a white solid.

¹H NMR (600 MHz, Chloroform-d) δ 8.00 (t, J=1.9 Hz, 1H), 7.73-7.70 (m, 2H), 7.68 (s, 1H), 7.61 (t, J=1.7 Hz, 1H), 7.60 (dd, J=2.2, 0.9 Hz, 1H), 7.49 (t, J=7.7 Hz, 1H), 7.47-7.43 (m, 2H), 7.39-7.35 (m, 1H), 7.08 (s, 1H), 6.99 (s, 1H), 6.60 (t, J=6.1 Hz, 1H), 4.08 (t, J=6.8 Hz, 2H), 3.51 (q, J=6.5 Hz, 3H), 2.15 (p, J=6.8 Hz, 2H).

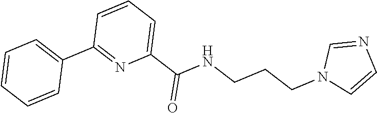 [SMILES: O=C(NCCCn1ccnc1)c1cccc(-c2ccccc2)n1]

### N-(3-(1H-imidazol-1-yl)propyl)-6-phenylpicolinamide

General Procedure 1 (Scheme 1): Flash chromatography: (EtOAc/MeOH=90/10). 18% yield as colorless oil.

¹H NMR (400 MHz, Chloroform-d) δ 8.76 (dd, J=2.3, 0.9 Hz, 1H), 8.25 (dd, J=8.1, 0.9 Hz, 1H), 8.17 (t, J=6.3 Hz, 1H), 8.04 (ddd, J=8.0, 2.3, 0.7 Hz, 1H), 7.73-7.58 (m, 3H), 7.53-7.48 (m, 2H), 7.47-7.44 (m, 1H), 7.15-6.97 (m, 2H), 4.08 (t, J=7.0 Hz, 2H), 3.53 (q, J=6.6 Hz, 2H), 2.16 (p, J=6.8 Hz, 2H).

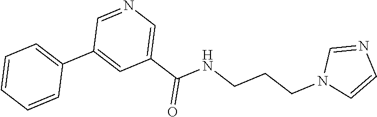 [SMILES: O=C(NCCCn1ccnc1)c1cncc(-c2ccccc2)c1]

### N-(3-(1H-imidazol-1-yl)propyl)-5-phenylnicotinamide

General Procedure 1 (Scheme 1): Flash chromatography: (EtOAc/MeOH=90/10). 32% yield as a white solid.

¹H NMR (400 MHz, Chloroform-d) δ 8.95 (d, J=38.1 Hz, 2H), 8.36 (q, J=2.1 Hz, 1H), 7.82 (t, J=5.6 Hz, 1H), 7.73 (s, 1H), 7.62-7.55 (m, 2H), 7.49-7.36 (m, 3H), 7.02 (d, J=20.4 Hz, 2H), 4.10 (t, J=6.6 Hz, 2H), 3.48 (q, J=6.4 Hz, 2H), 2.23-2.09 (m, 2H).

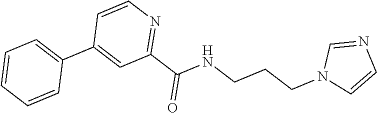 [SMILES: O=C(NCCCn1ccnc1)c1cc(-c2ccccc2)ccn1]

### N-(3-(1H-imidazol-1-yl)propyl)-4-phenylpicolinamide

General Procedure 1 (Scheme 1): Flash chromatography: (EtOAc/MeOH=90/10). 50% yield as a white solid.

¹H NMR (400 MHz, Chloroform-d) δ 8.57 (dq, J=5.1, 0.8 Hz, 1H), 8.43 (dq, J=1.5, 0.7 Hz, 1H), 8.24 (s, 1H), 7.93 (s, 1H), 7.73-7.67 (m, 2H), 7.65 (ddd, J=5.1, 1.9, 0.7 Hz, 1H), 7.53-7.42 (m, 3H), 7.13 (s, 1H), 7.07 (s, 1H), 4.15-4.10 (m, 2H), 3.53 (q, J=6.5 Hz, 2H), 2.16 (p, J=6.8 Hz, 3H).

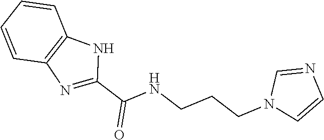 [SMILES: O=C(NCCCn1ccnc1)c1nc2ccccc2[nH]1]

### N-(3-(1H-imidazol-1-yl)propyl)-1H-benzo[d]imidazole-2-carboxamide

General Procedure 1 (Scheme 1): Flash chromatography: (EtOAc/MeOH=90/10). 65% yield as a white solid.

¹H NMR (600 MHz, Chloroform-d) δ 8.37 (t, J=7.8 Hz, 1H), 7.67 (s, 1H), 7.61 (s, 2H), 7.31 (dh, J=8.2, 4.1 Hz, 2H), 7.09 (s, 1H), 6.95 (s, 1H), 4.05 (td, J=7.0, 2.8 Hz, 2H), 3.51 (qd, J=6.3, 2.7 Hz, 2H), 3.48 (d, J=3.4 Hz, 1H), 2.12 (p, J=6.7 Hz, 2H).

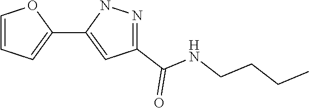 [SMILES: CCCCNC(=O)c1cc(-c2ccco2)[nH]n1]

### N-butyl-5-(furan-2-yl) isoxazole-3-carboxamide

General Procedure 1 (Scheme 1): Flash chromatography: (Hexane/EtOAc: up to 75% EtOAc). 42% yield as a white solid.

¹H NMR (400 MHz, Chloroform-d) δ 7.57 (dd, J=1.8, 0.7 Hz, 1H), 6.94 (d, J=3.5 Hz, 1H), 6.85 (s, 1H), 6.78 (s, 1H), 6.55 (dd, J=3.5, 1.8 Hz, 1H), 3.45 (q, J=7.3 Hz, 2H), 1.61 (p, J=7.4 Hz, 2H), 1.42 (p, J=7.4 Hz, 2H), 0.96 (t, J=7.3 Hz, 3H).

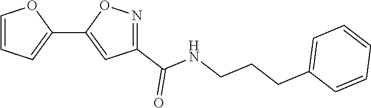 [SMILES: O=C(NCCCc1ccccc1)c1cc(-c2ccco2)on1]

### 5-(furan-2-yl)-N-(3-phenylpropyl)isoxazole-3-carboxamide

General Procedure 1 (Scheme 1): Flash chromatography: (Hexane/EtOAc: up to 100% EtOAc). 42% yield as a white solid.

¹H NMR (600 MHz, Chloroform-d) δ 7.58 (d, J=2.2 Hz, 1H), 7.46 (d, J=2.6 Hz, 1H), 7.41 (d, J=2.7 Hz, 1H), 6.95 (d, J=3.4 Hz, 1H), 6.90 (s, 1H), 6.85 (d, J=2.6 Hz, 1H), 6.56 (dt, J=4.3, 2.0 Hz, 1H), 4.15 (t, J=6.7 Hz, 3H), 3.47 (q, J=6.7 Hz, 3H), 1.95 (p, J=7.0 Hz, 3H), 1.62 (p, J=7.3 Hz, 3H). MS (ESI):297.13 [M+H]⁺.

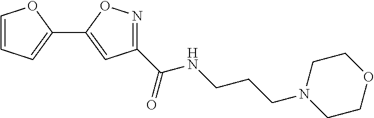 [SMILES: O=C(NCCCN1CCOCC1)c1cc(-c2ccco2)on1]

### 5-(furan-2-yl)-N-(3-morpholinopropyl)isoxazole-3-carboxamide

General Procedure 1 (Scheme 1): Flash chromatography: (EtOAc/MeOH=90/10). 22% yield as a pink solid.

¹H NMR (400 MHz, Chloroform-d) δ 8.63 (s, 1H), 7.54 (dd, J=1.8, 0.8 Hz, 1H), 6.91 (dd, J=3.5, 0.8 Hz, 1H), 6.82 (s, 1H), 6.55-6.50 (m, 1H), 3.80 (t, J=4.7 Hz, 4H), 3.55 (q, J=5.2 Hz, 2H), 2.53 (dd, J=14.9, 8.8 Hz, 6H), 1.77 (p, J=6.1 Hz, 3H).

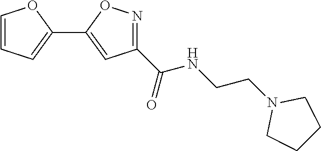 [SMILES: O=C(NCCN1CCCC1)c1cc(-c2ccco2)on1]

### 5-(furan-2-yl)-N-(2-(pyrrolidin-1-yl)ethyl)isoxazole-3-carboxamide

General Procedure 1 (Scheme 1): Flash chromatography: (EtOAc/MeOH=90/10). 16% yield as a pink solid.

¹H NMR (400 MHz, Chloroform-d) δ 7.57 (dt, J=1.8, 0.7 Hz, 1H), 6.94 (dt, J=3.5, 0.7 Hz, 1H), 6.86 (s, 1H), 6.55 (ddt, J=3.5, 1.8, 0.6 Hz, 1H), 3.58 (q, J=5.9 Hz, 2H), 2.74 (t, J=6.1 Hz, 2H), 2.60 (s, 4H), 1.82 (p, J=3.6 Hz, 5H).

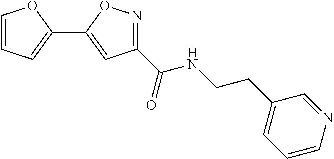 [SMILES: O=C(NCCc1cccnc1)c1cc(-c2ccco2)on1]

### 5-(Furan-2-yl)-N-(2-(pyridin-3-yl)ethyl)isoxazole-3-carboxamide

General Procedure 1 (Scheme 1): Flash chromatography: (Hexane/EtOAc: up to 100% EtOAc). 38% yield as a pale-yellow solid.

¹H NMR (600 MHz, Chloroform-d) δ 8.50 (s, 2H), 7.57 (m, 2H), 7.25 (dd, J=7.7, 5.0 Hz, 1H), 7.02 (t, J=5.6 Hz, 1H), 6.93 (d, J=3.5 Hz, 1H), 6.84 (s, 1H), 6.54 (dd, J=3.5, 1.8 Hz, 1H), 3.72 (q, J=6.9 Hz, 2H), 2.95 (t, J=7.2 Hz, 2H). MS (ESI):284.10 [M+H]⁺.

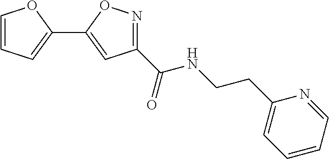 [SMILES: O=C(NCCc1ccccn1)c1cc(-c2ccco2)on1]

### 5-(Furan-2-yl)-N-(2-(pyridin-2-yl)ethyl)isoxazole-3-carboxamide

General Procedure 1 (Scheme 1): Flash chromatography: (Hexane/EtOAc=20/80). 52% yield as a pale-yellow solid.

¹H NMR (600 MHz, Chloroform-d) δ 8.58 (ddd, J=4.9, 1.9, 1.0 Hz, 1H), 7.75 (s, 1H), 7.62 (td, J=7.7, 1.8 Hz, 1H), 7.56 (dd, J=1.8, 0.8 Hz, 1H), 7.19 (d, J=7.8 Hz, 1H), 7.16 (ddd, J=7.6, 4.8, 1.1 Hz, 1H), 6.92 (dd, J=3.5, 0.9 Hz, 1H), 6.84 (s, 1H), 6.54 (dd, J=3.5, 1.8 Hz, 1H), 3.89 (q, J=6.2 Hz, 2H), 3.11 (t, J=6.4 Hz, 2H). MS (ESI):284.10[M+H]⁺.

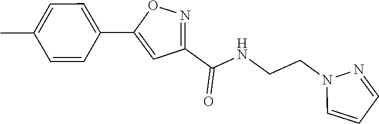 [SMILES: Cc1ccc(-c2cc(C(=O)NCCn3cccn3)no2)cc1]

### N-(2-(1H-pyrazol-1-yl)ethyl)-5-(p-tolyl)isoxazole-3-carboxamide

General Procedure 1 (Scheme 1): Flash chromatography: (Hexane/EtOAc=30/70). 83% yield as a pale-yellow solid.

¹H NMR (600 MHz, Chloroform-d) δ 7.74 (d, J=8.2 Hz, 2H), 7.64 (d, J=1.9 Hz, 1H), 7.51 (t, J=6.0 Hz, 1H), 7.48 (d, J=2.3 Hz, 1H), 7.34 (d, J=8.9 Hz, 2H), 6.96 (s, 1H), 6.33 (t, J=2.1 Hz, 1H), 4.44 (t, J=5.6 Hz, 2H), 3.98 (q, J=6.0 Hz, 2H), 2.47 (s, 3H). MS (ESI):197.14[M+H]⁺.

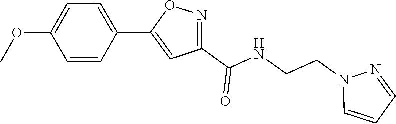 [SMILES: COc1ccc(-c2cc(C(=O)NCCn3cccn3)no2)cc1]

### N-(2-(1H-pyrazol-1-yl)ethyl)-5-(4-methoxyphenyl)isoxazole-3-carboxamide

General Procedure 1 (Scheme 1): Flash chromatography: (Hexane/EtOAc=30/70). 79% yield as a white solid.

¹H NMR (600 MHz, Chloroform-d) δ 7.71 (d, J=9.0 Hz, 2H), 7.56 (d, J=4.8 Hz, 1H), 7.43 (t, J=5.9 Hz, 1H), 7.40 (d, J=4.2 Hz, 1H), 6.97 (d, J=9.1 Hz, 2H), 6.82 (s, 1H), 6.25 (t, J=4.0 Hz, 1H), 4.37 (t, J=7.2 Hz, 2H), 3.91 (p, J=5.6 Hz, 2H), 3.85 (s, 3H).

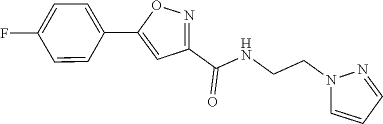 [SMILES: O=C(NCCn1cccn1)c1cc(-c2ccc(F)cc2)on1]

### N-(2-(1H-pyrazol-1-yl)ethyl)-5-(4-fluorophenyl)isoxazole-3-carboxamide

General Procedure 1 (Scheme 1): Flash chromatography: (Hexane/EtOAc=30/70). 65% yield as a white solid.

¹H NMR (600 MHz, Chloroform-d) δ 7.78 (dd, J=8.8, 5.2 Hz, 2H), 7.59 (d, J=1.9 Hz, 1H), 7.41 (d, J=2.3 Hz, 1H), 7.40 (s, 1H), 7.18 (t, J=8.5 Hz, 2H), 6.90 (s, 1H), 6.28 (t, J=2.1 Hz, 1H), 4.38 (m, 2H), 3.93 (q, J=5.7 Hz, 2H). MS (ESI): 301.11 [M+H]⁺.

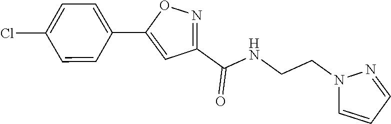 [SMILES: O=C(NCCn1cccn1)c1cc(-c2ccc(Cl)cc2)on1]

### N-(2-(1H-pyrazol-1-yl)ethyl)-5-(4-chlorophenyl)isoxazole-3-carboxamide

General Procedure 1 (Scheme 1): Flash chromatography: (Hexane/EtOAc=20/80). 32% yield as a pale-yellow solid.

¹H NMR (600 MHz, Chloroform-d) δ 7.72 (d, J=8.7 Hz, 2H), 7.58 (d, J=1.6 Hz, 1H), 7.46 (d, J=8.8 Hz, 2H), 7.41 (m, 2H), 6.94 (s, 1H), 6.27 (t, J=2.1 Hz, 1H), 4.38 (t, J=5.7 Hz, 2H), 3.93 (q, J=5.8 Hz, 2H). MS (ESI):317.08[M+H]⁺.

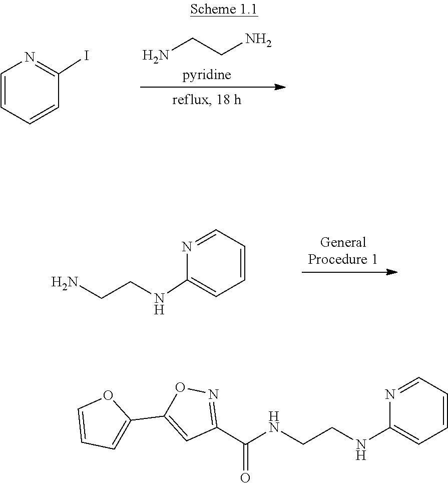

### 5-(furan-2-yl)-N-(2-(pyridin-2-ylamino)ethyl)isoxazole-3-carboxamide

To a solution of 2-iodopyridine (101 μL, 0.98 mmol) in pyridine (2.5 mL), ethylenediamine (326 μL, 4.88 mmol) was added. The mixture was heated at 115° C. (reflux) for 18 hours. After cooling to room temperature, excess of ethylenediamine and solvent were evaporated under reduced pressure. The crude residue was directly used for second step without further purification. General Procedure 1 (Scheme 1): 13% yield of two steps as a pale-yellow solid. Flash chromatography: (100% EtOAc).

¹H NMR (400 MHz, Chloroform-d) δ 8.42 (s, 1H), 8.14 (d, J=4.2 Hz, 1H), 7.56 (dd, J=1.8, 0.8 Hz, 1H), 7.41 (ddd, J=8.5, 7.1, 1.9 Hz, 1H), 6.93 (d, J=3.4 Hz, 1H), 6.84 (s, 1H), 6.61 (ddd, J=7.1, 5.2, 0.9 Hz, 1H), 6.55 (dd, J=3.5, 1.8 Hz, 1H), 6.47 (d, J=8.4 Hz, 1H), 5.05 (s, 1H), 3.67 (m, 5H).

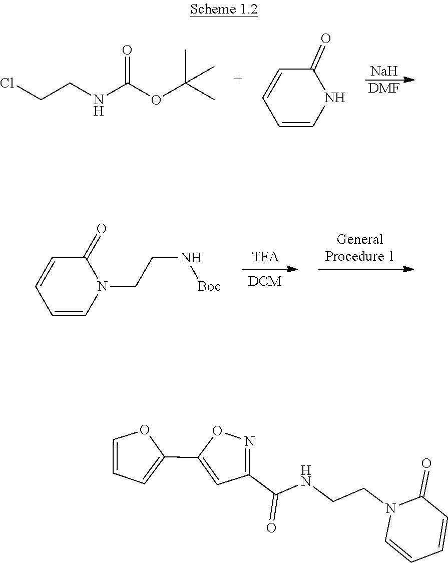

### tert-butyl (2-(2-oxopyridin-1(2H)-yl)ethyl)carbamate

To a solution of pyridine-2(1H)-one (155 mg, 1.62 mmol) in DMF at 0° C. was added sodium hydride (58.7 mg, 2.44 mmol). The mixture was stirred at 80° C. under nitrogen for 2 hours and tert-butyl (2-chloroethyl)carbamate was added slowly. The resulting mixture was stirred at 80° C. for 16 hours before being poured into DIW and extracted with EtOAc three times. Combined organic was washed with DIW, saturated brine and dried with Na₂SO₄. Solvent was removed in vacuo to yield a white solid. White solid was washed with diethyl ether twice and collected as crude for subsequent steps.

### 5-(furan-2-yl)-N-(2-(2-oxopyridin-1(2H)-yl)ethyl)isoxazole-3-carboxamide

tert-butyl (2-(2-oxopyridin-1(2H)-yl)ethyl)carbamate crude was dissolved in TFA:DCM (1:1) solution at 0° C. The mixture was allowed to warm to room temperature and stirred for 4 hours. Solvent was removed in vacuo. Resulting residue was used following General Procedure 1 (Scheme 1). Flash chromatography (EtOAc/MeOH: up to 20% MeOH) to yield 18.1% over 3 steps as an off-white solid.

1H NMR (600 MHz, Chloroform-d) δ 7.69 (t, J=5.8 Hz, 1H), 7.55 (dd, J=1.9, 0.7 Hz, 1H), 7.33 (ddd, J=8.9, 6.6, 2.0 Hz, 1H), 7.26 (dd, J=6.7, 1.9 Hz, 1H), 6.92 (dd, J=3.5, 0.7 Hz, 1H), 6.81 (s, 1H), 6.58 (dt, J=9.1, 0.9 Hz, 1H), 6.53 (dd, J=3.5, 1.8 Hz, 1H), 6.15 (td, J=6.7, 1.3 Hz, 1H), 4.22-4.15 (m, 2H), 3.80 (q, J=5.9 Hz, 2H).

General Procedure 2: Amide Formation from Ester

### General Procedure 2.1: Hydrolysis Followed by T3P Amide Coupling

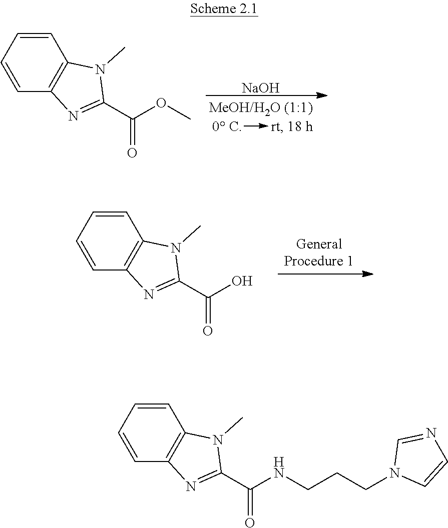

### N-(3-(1H-imidazol-1-yl)propyl)-1-methyl-1H-benzo[d]imidazole-2-carboxamide

To the solution of methyl 1-methyl-1H-benzo[d]imidazole-2-carboxylate (118 mg, 0.62 mmol) in methanol (0.75 mL) and water (0.75 mL), sodium hydroxide (50 mg, 1.24 mmol) was added at 0° C. The mixture was stirred at room temperature for 18 hours. The reaction was concentrated to remove methanol and diluted with water. After acidifying to pH 2, the mixture was cooled over ice, during which white solid was formed. The solid was filtered and dried on high vacuum to give 1-methyl-1H-benzo[d]imidazole-2-carboxylic acid as a white fluffy solid. The acid was used for amide formation (General Procedure 1) to provide the title compound (30 mg, 17% yield of two steps). Flash chromatography: (EtOAc/MeOH=90/10).

¹H NMR (400 MHz, Chloroform-d) δ 8.04 (t, J=5.8 Hz, 1H), 7.76 (d, J=7.7 Hz, 1H), 7.71 (s, 1H), 7.40 (m, 3H), 7.10 (s, 2H), 4.23 (s, 3H), 4.09 (t, J=7.0 Hz, 2H), 3.49 (q, J=6.6 Hz, 2H), 2.15 (p, J=6.8 Hz, 2H).

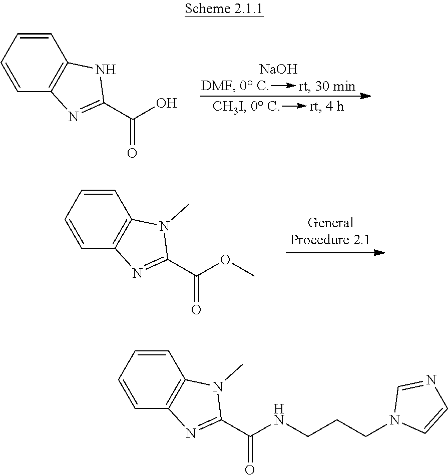

### Methyl 1-methyl-1H-benzo[d]imidazole-2-carboxylate

To a solution of 1H-benzo[d]imidazole-2-carboxylic acid (100 mg, 0.62 mmol) in DMF (1.5 mL), sodium hydride (60% dispersion in mineral oil) (62 mg, 1.54 mmol) was added slowly at 0° C. The mixture was warmed to room temperature and stirred for 30 minutes. After cooling to 0° C., iodomethane (192 μL, 3.08 mmol) was added dropwise. After 4 hours at room temperature, the reaction was quenched with saturated ammonium chloride. The aqueous layer was extracted with EtOAc three times. The combined organic layers were dried over NaSO₄, filtered, and evaporated. The crude material was purified via flash column chromatography (Hexane/EtOAc=60/40) to provide the methylated compound (73 mg, 63% yield) as a white solid, which was used for amide bond formation reaction via General Procedure 2.1 (Scheme 2.1).

¹H NMR (400 MHz, Chloroform-d) δ 7.90 (dt, J=8.1, 1.0 Hz, 1H), 7.45 (m, 2H), 7.37 (ddd, J=8.2, 5.4, 2.9 Hz, 1H), 4.19 (s, 3H), 4.05 (s, 3H).

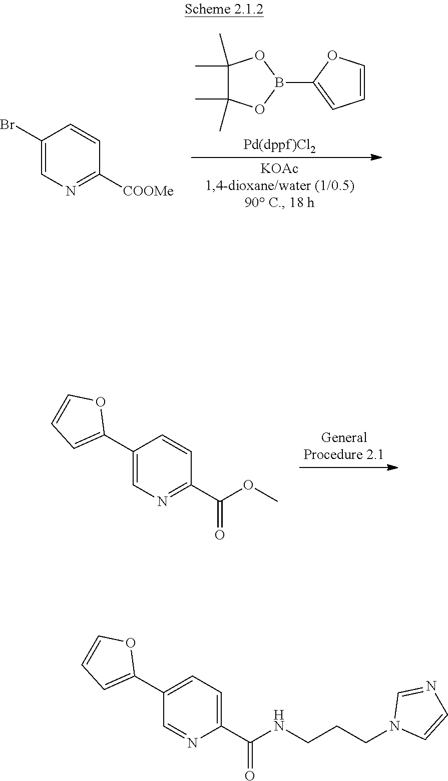

### Methyl 5-(furan-2-yl)picolinate

To a solution of methyl 5-bromopicolinate (50 mg, 0.23 mmol) in 1,4-dioxane (0.6 mL) and water (0.3 mL), furanyl dioxaborolane (52.17 μL, 0.28 mmol), potassium acetate (45 mg, 0.46 mmol) and (1,1′-Bis(diphenylphosphino)ferrocene)palladium(II) dichloride (Pd(dppf)Cl₂) (19 mg, 0.02 mmol, 10 mol %) were added. The mixture was heated at 90° C. for 18 hours. After cooling to room temperature, the solvent was evaporated, and the residue was dissolved in DCM. The organics were washed with water three times, dried over NaSO₄, filtered, and evaporated. The crude material was purified via flash column chromatography (Hexane/EtOAc=50/50) to provide the title compound (36 mg, 77% yield) as a white solid.

¹H NMR (400 MHz, Chloroform-d) δ 9.00 (dd, J=2.3, 0.8 Hz, 1H), 8.13 (dd, J=8.3, 0.8 Hz, 1H), 8.03 (dd, J=8.2, 2.2 Hz, 1H), 7.56 (dd, J=1.8, 0.7 Hz, 1H), 6.88 (dd, J=3.5, 0.7 Hz, 1H), 6.53 (dd, J=3.5, 1.8 Hz, 1H), 4.00 (s, 3H).

### N-(3-(1H-imidazol-1-yl)propyl)-5-(furan-2-yl)picolinamide

Methyl 5-(furan-2-yl)picolinate was used for amide bond formation reaction via General Procedure 2.1 to provide the title compound as a pale-yellow solid (54% yield of two steps). Flash chromatography: (EtOAc/MeOH=90/10).

¹H NMR (400 MHz, Chloroform-d) δ 8.84 (dd, J=2.2, 0.8 Hz, 1H), 8.19 (dd, J=8.2, 0.8 Hz, 1H), 8.07 (m, 2H), 7.57 (m, 2H), 7.09 (s, 1H), 7.00 (s, 1H), 6.85 (dd, J=3.4, 0.7 Hz, 1H), 6.54 (dd, J=3.4, 1.8 Hz, 1H), 4.06 (t, J=7.0 Hz, 2H), 3.51 (q, J=6.6 Hz, 2H), 2.15 (p, J=6.8 Hz, 2H).

General Procedure 2.2: Direct Amide Formation with Trimethylaluminum (TMA) Activation

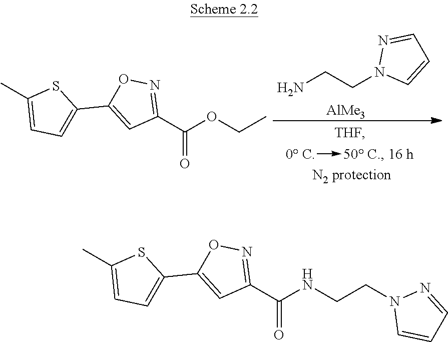

### N-(2-(1H-pyrazol-1-yl)ethyl)-5-(5-methylthiophen-2-yl)isoxazole-3-carboxamide

To a solution of ethyl 5-(5-methylthiophen-2-yl)isoxazole-3-carboxylate (30 mg, 0.13 mmol) and 2-(1H-pyrazol-1-yl)ethan-1-amine (16 mg, 0.14 mmol) in THF (1 mL), trimethylaluminum (2M in toluene) (128 μL, 0.26 mmol) was added at 0° C. under N₂ balloon protection. The mixture was heated at 50° C. for 16 hours, during which pale-yellow gel was formed. After cooling to room temperature, the reaction mixture was dissolved in EtOAc. The organics was washed with brine, dried over NaSO₄, filtered, and evaporated. The crude material was purified via flash column chromatography (100% EtOAc) to provide the title compound (27 mg, 68% yield) as a white solid.

¹H NMR (600 MHz, Chloroform-d) δ 7.56 (d, J=1.9 Hz, 1H), 7.39 (d, J=2.2 Hz, 1H), 7.35 (t, J=5.9 Hz, 1H), 7.32 (d, J=3.6 Hz, 1H), 6.78 (d, J=3.7 Hz, 1H), 6.70 (s, 1H), 6.25 (t, J=2.2 Hz, 1H), 4.36 (t, J=5.8 Hz, 2H), 3.90 (q, J=5.8 Hz, 2H), 2.53 (s, 3H). MS (ESI):303.09[M+H]⁺.

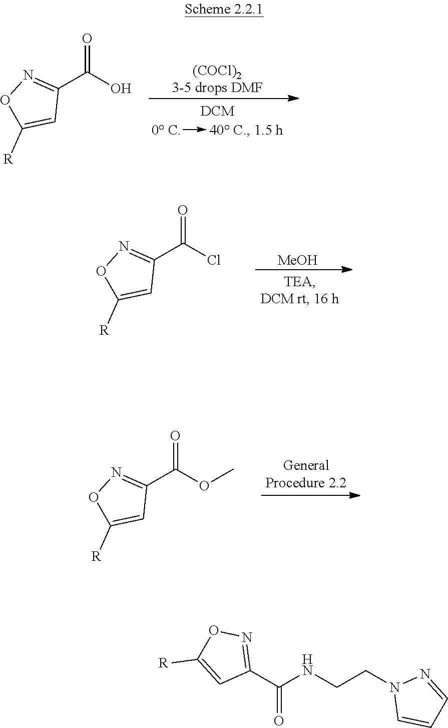

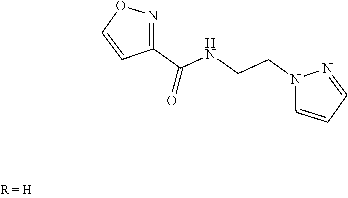 [SMILES: O=C(NCCn1cccn1)c1ccon1]

### Methyl isoxazole-3-carboxylate

To a solution of isoxazole-3-carboxylic acid (75 mg, 0.66 mmol) in DCM (2 mL), 3-5 drops DMF was added. Then oxalyl chloride (2M in DCM) (1.0 mL, 1.99 mmol) was added dropwise at 0° C. The reaction was heated at 40° C. for 1.5 hours. After cooling to room temperature, the solvent and excess oxalyl chloride were removed under reduced pressure. The residue was dissolved in DCM (4 mL). The resulting mixture was neutralized with triethylamine (306 μL, 2.19 mmol) at 0° C. to pH 6-7 and tested by pH paper. Methanol (35 μL, 0.86 mmol) was added. After stirring at room temperature for 16 hours, the reaction was washed with saturated NaHCO₃ and water, dried over NaSO₄, filtered, and evaporated. The crude material was purified via flash column chromatography (Hexane/EtOAc: 10-20% EtOAc) to provide the title compound (60 mg, 71% yield).

¹H NMR (400 MHz, Chloroform-d) 8.52 (d, J=1.7 Hz, 1H), 6.76 (d, J=1.7 Hz, 1H), 3.95 (s, 3H).

### N-(2-(1H-pyrazol-1-yl)ethyl)isoxazole-3-carboxamide

Methyl isoxazole-3-carboxylate (30 mg, 0.21 mmol) was used for amide bond formation reaction via General Procedure 2.2 to provide title compound as a white solid (34 mg, 79% yield). Flash chromatography: (100% EtOAc).

¹H NMR (400 MHz, Chloroform-d) δ 8.45 (d, J=1.7 Hz, 1H), 7.55 (d, J=1.9 Hz, 1H), 7.44 (s, 1H), 7.39 (d, J=2.3 Hz, 1H), 6.80 (d, J=1.6 Hz, 1H), 6.25 (t, J=2.1 Hz, 1H), 4.35 (t, J=5.5 Hz, 2H), 3.89 (q, J=5.9 Hz, 2H).

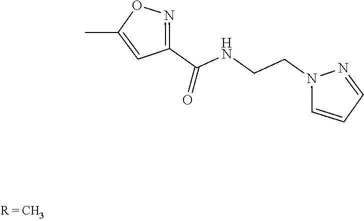 [SMILES: Cc1cc(C(=O)NCCn2cccn2)no1]

### N-(2-(1H-pyrazol-1-yl)ethyl)-5-methylisoxazole-3-carboxamide

Methyl 5-methylisoxazole-3-carboxylate (40 mg, 0.26 mmol) was synthesized by the same method (Scheme 2.2.1) (55% yield) and was used for amide bond formation reaction via General Procedure 2.2 to provide the title compound (53 mg, 53% yield) as a white solid. Flash chromatography: (100% EtOAc).

¹H NMR (400 MHz, Chloroform-d) δ 7.55 (d, J=1.9 Hz, 1H), 7.38 (d, J=2.3 Hz, 1H), 7.29 (s, 1H), 6.41 (s, 1H), 6.24 (t, J=2.1 Hz, 1H), 4.34 (t, J=5.5 Hz, 2H), 3.87 (q, J=5.9 Hz, 2H), 2.46 (s, 3H).

### General Procedure 3: Gabriel Synthesis for Amine Precursors

### General Procedure 3.1: Use of Crude Amine Product

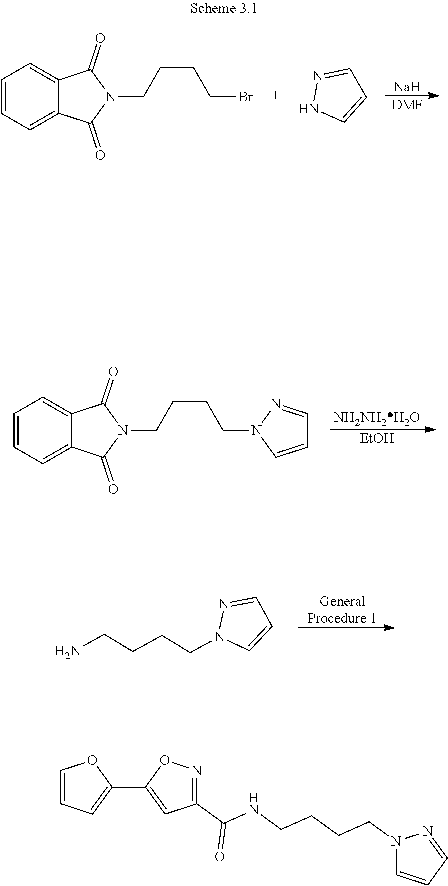

### 2-(4-(1H-pyrazol-1-yl)butyl)isoindoline-1,3-dione

To a solution of pyrazole (276 mg, 4.05 mmol) in DMF at 0° C. was added sodium hydride (60% in paraffin) (97.1 mg, 4.05 mmol). The solution was stirred at room temperature for 1 hour and cooled to 0° C. To the cool solution, N-(4-Bromobutyl)phthalimide (1.00 g, 3.52 mmol) in DMF was added dropwise and the solution was heated 90° C. for 16 hours. The resulting mixture was extracted with EtOAc. Combined organic was washed with water and brine, dry with Na₂SO₄, and filtered. Organic crude was purified using flash column chromatography (Hexane/EtOAc: up to 75% EtOAc) to yield a white solid (312 mg, 32.9%).

¹H NMR (400 MHz, Chloroform-d) δ 7.81 (dd, J=5.4, 3.1 Hz, 2H), 7.69 (dd, J=5.4, 3.0 Hz, 2H), 7.46-7.44 (m, 1H), 7.36 (d, J=2.1 Hz, 1H), 6.20 (t, J=2.1 Hz, 1H), 4.16 (t, J=7.0 Hz, 2H), 3.68 (t, J=7.1 Hz, 2H), 1.89 (p, J=6.9 Hz, 2H), 1.65 (p, J=7.7 Hz, 2H). MS (ESI):301.13[M+H]⁺.

### N-(4-(1H-pyrazol-1-yl)butyl)-5-(furan-2-yl)isoxazole-3-carboxamide

To a solution of 2-(4-(1H-pyrazol-1-yl)butyl)isoindoline-1,3-dione (250, 0.928 mmol) in 200 proof EtOH was added hydrazine monohydrate (60% w/w in H₂O) (139 mg, 2.78 mmol). The solution was refluxed for 16 hours. Reaction mixture was filtered through celite and concentrated in vacuo to afford a yellow oil (183 mg, quant.). Crude oil was used for subsequent amide coupling (Scheme 1) without further purification. Flash column chromatography (Hexane/EtOAc: up to 100% EtOAc) yield yellow solid (39.8 mg, 19%)

¹H NMR (400 MHz, Chloroform-d) δ 7.55 (dd, J=1.8, 0.7 Hz, 1H), 7.50 (dd, J=1.9, 0.7 Hz, 1H), 7.37 (dd, J=2.3, 0.7 Hz, 1H), 6.95-6.88 (m, 2H), 6.83 (s, 1H), 6.53 (dd, J=3.5, 1.8 Hz, 1H), 6.23 (t, J=2.1 Hz, 1H), 4.17 (t, J=6.9 Hz, 2H), 3.44 (q, J=7.2 Hz, 2H), 1.95 (dt, J=8.5, 7.1 Hz, 2H), 1.60 (p, J=7.6 Hz, 2H).

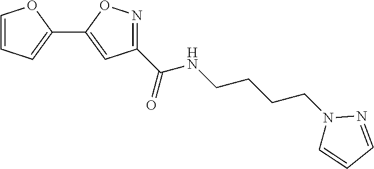 [SMILES: O=C(NCCCCn1cccn1)c1cc(-c2ccco2)on1]

### N-(4-(1H-pyrazol-1-yl)butyl)-5-(thiophen-2-yl)isoxazole-3-carboxamide

General Procedure 3.1 (Scheme 3.1): Flash chromatography (Hexane/EtOAc: up to 100% EtOAc). 19.9% yield as a white solid.

¹H NMR (400 MHz, Chloroform-d) δ 7.51 (dt, J=3.7, 0.8 Hz, 1H), 7.49 (d, J=1.9 Hz, 1H), 7.46 (dt, J=5.0, 0.8 Hz, 1H), 7.37 (d, J=2.2 Hz, 1H), 7.12 (ddd, J=5.0, 3.7, 0.5 Hz, 1H), 6.95 (d, J=6.3 Hz, 1H), 6.78 (d, J=0.5 Hz, 1H), 6.22 (t, J=2.0 Hz, 1H), 4.17 (t, J=6.9 Hz, 2H), 3.44 (q, J=6.9 Hz, 2H), 1.95 (p, J=6.8 Hz, 2H), 1.60 (p, J=7.4 Hz, 2H).

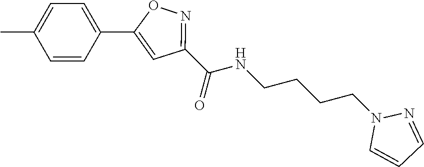 [SMILES: Cc1ccc(-c2cc(C(=O)NCCCCn3cccn3)no2)cc1]

### N-(4-(1H-pyrazol-1-yl)butyl)-5-phenylisoxazole-3-carboxamide

General Procedure 3.1 (Scheme 3.1): Flash chromatography (Hexane/EtOAc: up to 100% EtOAc). 11.4% yield as a white solid.

¹H NMR (400 MHz, Chloroform-d) δ 7.82-7.77 (m, 2H), 7.53 (dd, J=1.9, 0.7 Hz, 1H), 7.50-7.46 (m, 3H), 7.40 (dd, J=2.3, 0.7 Hz, 1H), 6.98-6.91 (m, 2H), 6.26 (t, J=2.1 Hz, 1H), 4.21 (t, J=6.9 Hz, 2H), 3.47 (q, J=7.0 Hz, 2H), 1.99 (p, J=7.0 Hz, 2H), 1.63 (p, J=7.5 Hz, 2H).

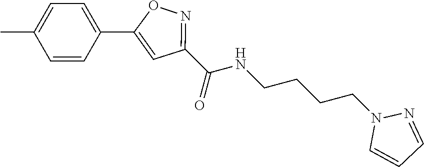 [SMILES: Cc1ccc(-c2cc(C(=O)NCCCCn3cccn3)no2)cc1]

### N-(4-(1H-pyrazol-1-yl)butyl)-5-(p-tolyl)isoxazole-3-carboxamide

General Procedure 3.1 (Scheme 3.1): Flash chromatography (Hexane/EtOAc: up to 75% EtOAc). 11.6% yield as a white solid.

¹H NMR (600 MHz, Chloroform-d) δ 7.66 (d, J=8.2 Hz, 2H), 7.50 (s, 1H), 7.41-7.36 (m, 1H), 7.27 (d, J=8.1 Hz, 2H), 6.94 (s, 1H), 6.88 (s, 1H), 6.26-6.22 (m, 1H), 4.18 (t, J=6.9 Hz, 2H), 3.45 (q, J=7.1 Hz, 2H), 2.40 (s, 3H), 1.96 (p, J=7.0 Hz, 2H), 1.61 (p, J=7.3 Hz, 2H). MS (ESI):325.16[M+H]⁺.

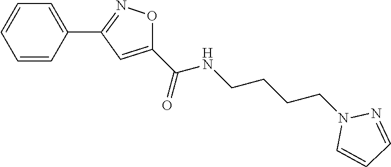 [SMILES: O=C(NCCCCn1cccn1)c1cc(-c2ccccc2)no1]

### N-(4-(1H-pyrazol-1-yl)butyl)-3-phenylisoxazole-5-carboxamide

General Procedure 3.1 (Scheme 3.1): Flash chromatography (Hexane/EtOAc: up to 100% EtOAc). 6.0% yield as a white solid.

¹H NMR (600 MHz, Chloroform-d) δ 7.82 (ddt, J=5.4, 2.8, 1.6 Hz, 2H), 7.54 (s, 1H), 7.48 (dtt, J=5.5, 3.5, 1.8 Hz, 3H), 7.40 (s, 1H), 7.21 (d, J=1.3 Hz, 1H), 6.91 (s, 1H), 6.26 (t, J=2.1 Hz, 1H), 4.21 (t, J=6.8 Hz, 2H), 3.48 (q, J=7.0 Hz, 2H), 1.99 (p, J=6.8 Hz, 2H), 1.65 (p, J=7.1 Hz, 2H). MS (ESI):311.15 [M+H]⁺.

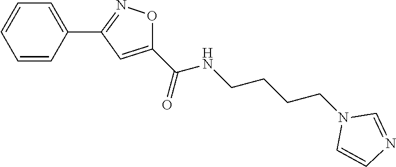 [SMILES: O=C(NCCCCn1ccnc1)c1cc(-c2ccccc2)no1]

### N-(4-(1H-imidazol-1-yl)butyl)-3-phenylisoxazole-5-carboxamide

General Procedure 3.1 (Scheme 3.1): Flash chromatography (EtOAc/MeOH=80/20). 8.2% yield as white oil.

¹H NMR (600 MHz, Chloroform-d) δ 7.87-7.81 (m, 2H), 7.75 (s, 1H), 7.51 (t, J=2.9 Hz, 3H), 7.26 (s, 1H), 7.14 (s, 1H), 7.01-6.94 (m, 2H), 4.07 (t, J=7.1 Hz, 3H), 3.53 (q, J=6.8 Hz, 3H), 1.93 (p, J=7.2 Hz, 3H), 1.68 (p, J=7.2 Hz, 3H). MS (ESI):311.15 [M+H]⁺.

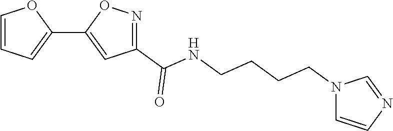 [SMILES: O=C(NCCCCn1ccnc1)c1cc(-c2ccco2)on1]

### N-(4-(1H-imidazol-1-yl)butyl)-5-(furan-2-yl)isoxazole-3-carboxamide

General Procedure 3.1 (Scheme 3.1): Flash chromatography (Hexane/EtOAc: up to 75% EtOAc). 26.3% yield as a yellow solid.

¹H NMR (400 MHz, Chloroform-d) δ 7.70 (s, 1H), 7.58 (dd, J=1.8, 0.8 Hz, 1H), 7.13 (s, 1H), 6.95 (t, J=3.8 Hz, 1H), 6.91 (s, 1H), 6.86 (s, 1H), 6.57-6.55 (m, 1H), 4.05 (t, J=7.1 Hz, 2H), 3.49 (q, J=6.8 Hz, 2H), 1.89 (p, J=7.2 Hz, 2H), 1.64 (p, J=7.1 Hz, 2H). MS (ESI):301.13[M+H]⁺.

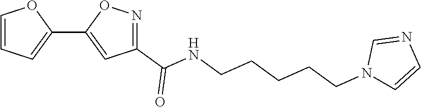 [SMILES: O=C(NCCCCCn1ccnc1)c1cc(-c2ccco2)on1]

### N-(5-(1H-imidazol-1-yl)pentyl)-5-(furan-2-yl)isoxazole-3-carboxamide

General Procedure 3.1 (Scheme 3.1): Flash chromatography (EtOAc/MeOH=80/20). 16.9% yield as a yellow solid.

¹H NMR (400 MHz, Chloroform-d) δ 7.57 (dd, J=1.8, 0.8 Hz, 1H), 7.54 (s, 1H), 7.07 (s, 1H), 6.95 (dd, J=3.5, 0.8 Hz, 1H), 6.93-6.87 (m, 2H), 6.85 (s, 1H), 6.55 (dd, J=3.5, 1.8 Hz, 1H), 3.96 (t, J=7.1 Hz, 2H), 3.44 (q, J=7.3 Hz, 2H), 1.84 (p, J=7.6 Hz, 2H), 1.65 (p, J=7.2 Hz, 2H), 1.39 (p, J=7.8 Hz, 2H). MS (ESI):315.14 [M+H]⁺.

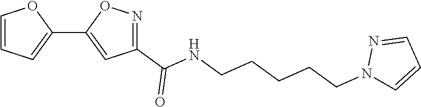 [SMILES: O=C(NCCCCCn1cccn1)c1cc(-c2ccco2)on1]

### N-(5-(1H-pyrazol-1-yl)pentyl)-5-(furan-2-yl)isoxazole-3-carboxamide

General Procedure 3.1 (Scheme 3.1): Flash chromatography (Hexane/EtOAc: up to 75% EtOAc). 27.3% yield as a white solid.

¹H NMR (400 MHz, Chloroform-d) δ 7.57 (dd, J=1.8, 0.8 Hz, 1H), 7.51 (dd, J=1.9, 0.7 Hz, 1H), 7.38 (d, J=2.3 Hz, 1H), 6.95 (dd, J=3.5, 0.8 Hz, 1H), 6.85 (s, 2H), 6.56 (dd, J=3.5, 1.8 Hz, 1H), 6.24 (t, J=2.1 Hz, 1H), 4.16 (t, J=7.0 Hz, 3H), 3.44 (q, J=7.0 Hz, 3H), 1.92 (p, J=7.8 Hz, 3H), 1.65 (p, J=7.6 Hz, 2H), 1.38 (p, J=7.7 Hz, 2H). MS (ESI):315.14[M+H]⁺.

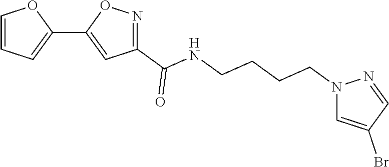 [SMILES: O=C(NCCCCn1cc(Br)cn1)c1cc(-c2ccco2)on1]

### N-(4-(4-bromo-1H-pyrazol-1-yl)butyl)-5-(furan-2-yl)isoxazole-3-carboxamide

General Procedure 3.1 (Scheme 3.1): Flash chromatography (Hexane/EtOAc: up to 75% EtOAc). 14% yield as a white solid.

¹H NMR (600 MHz, Chloroform-d) δ 7.58 (d, J=2.2 Hz, 1H), 7.46 (d, J=2.6 Hz, 1H), 7.41 (d, J=2.7 Hz, 1H), 6.95 (d, J=3.4 Hz, 1H), 6.90 (s, 1H), 6.85 (d, J=2.6 Hz, 1H), 6.56 (dt, J=4.3, 2.0 Hz, 1H), 4.15 (t, J=6.7 Hz, 3H), 3.47 (q, J=6.7 Hz, 3H), 1.95 (p, J=7.0 Hz, 3H), 1.62 (p, J=7.3 Hz, 3H). MS (ESI):379.04[M+H]⁺.

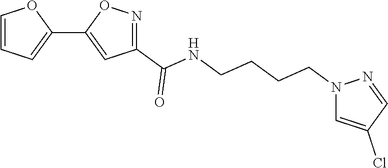 [SMILES: O=C(NCCCCn1cc(Cl)cn1)c1cc(-c2ccco2)on1]

### N-(4-(4-chloro-1H-pyrazol-1-yl)butyl)-5-(furan-2-yl)isoxazole-3-carboxamide

General Procedure 3.1 (Scheme 3.1): Flash chromatography (Hexane/EtOAc: up to 75% EtOAc). 21.3% yield as a white solid.

¹H NMR (600 MHz, Chloroform-d) δ 7.56 (d, J=1.9 Hz, 1H), 7.42 (s, 1H), 7.38 (s, 1H), 7.00-6.91 (m, 2H), 6.85 (s, 1H), 6.55 (dd, J=3.5, 1.8 Hz, 1H), 4.12 (t, J=6.9 Hz, 2H), 3.47 (q, J=7.1 Hz, 2H), 1.94 (p, J=6.9 Hz, 2H), 1.62 (p, J=7.4 Hz, 2H). MS (ESI):335.09[M+H]⁺.

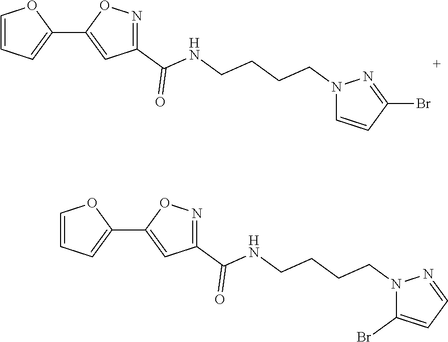 [SMILES: O=C(NCCCCn1ccc(Br)n1)c1cc(-c2ccco2)on1 ; O=C(NCCCCn1nccc1Br)c1cc(-c2ccco2)on1]

### N-(4-(3-bromo-1H-pyrazol-1-yl)butyl)-5-(furan-2-yl)isoxazole-3-carboxamide & N-(4-(5-bromo-1H-pyrazol-1-yl)butyl)-5-(furan-2-yl)isoxazole-3-carboxamide (2:1 Ratio)

General Procedure 3.1 (Scheme 3.1): 5-bromo-1H-pyrazole was used as starting material. Flash chromatography (Hexane/EtOAc: up to 100% EtOAc). 18.2% yield mixture as a pink solid.

3-bromo: ¹H NMR (400 MHz, Chloroform-d) δ 7.56 (dd, J=1.8, 0.8 Hz, 1H), 7.48 (d, J=1.9 Hz, 1H), 6.98 (s, 1H), 6.93 (d, J=3.6 Hz, 1H), 6.83 (s, 1H), 6.57-6.51 (m, 1H), 6.26 (d, J=1.9 Hz, 1H), 4.11 (dt, J=10.5, 7.0 Hz, 2H), 3.49-3.41 (m, 2H), 1.94 (p, J=7.1 Hz, 2H), 1.68-1.56 (m, 2H).

5-bromo: ¹H NMR (400 MHz, Chloroform-d) δ 7.56 (dd, J=1.8, 0.8 Hz, 1H), 7.28 (d, J=2.3 Hz, 1H), 6.93 (bd, J=3.6 Hz, 2H), 6.83 (s, 1H), 6.57-6.52 (m, 1H), 6.23 (d, J=2.2 Hz, 1H), 4.11 (dt, J=10.5, 7.0 Hz, 2H), 3.46 (qd, J=6.9, 3.5 Hz, 2H), 1.94 (p, J=7.1 Hz, 2H), 1.67-1.57 (m, 2H).

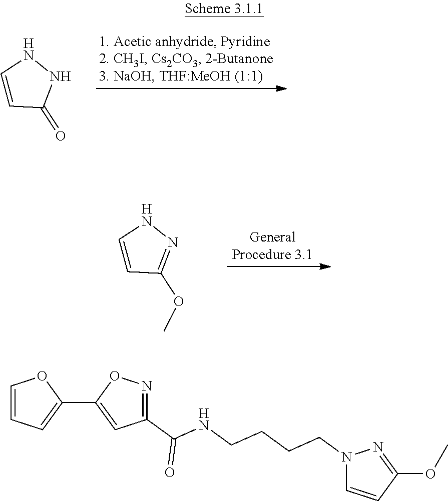

### 5-(furan-2-yl)-N-(4-(3-methoxy-1H-pyrazol-1-yl)butyl)isoxazole-3-carboxamide

To a solution of pyrazol-3-ol (253 mg, 3.00 mmol) in pyridine was added acetic anhydride (287 μL, 3.00 mmol) in pyridine dropwise. Resulting solution was stirred at 95° C. for 2 hours. Volatiles were removed in vacuo to yield dark yellow solid. Dry solid under high vacuum. Solid were dissolved with 2-Butanone. To the solution was added Iodomethane (843 μL, 13.5 mmol) and Cs₂CO₃ (1.03 g, 3.15 mmol). The resulting mixture was refluxed for 3 hours then filtered through celite. EtOAc was used to wash reaction flask. EtOAc was added to filtrate until no more precipitation form. Filtrate was filtered through celite then concentrated in vacuo to give a orange oil. Crude oil was taken up in a 1:1 mixture of THF:MeOH and 10 M aqueous NaOH (0.2 mL) and stirred at room temperature for 30 min. The reaction mixture was extracted with EtOAc. Combined organic was dried with Na₂SO₄, filtered, and concentrated in vacuo to afford 3-methoxy-1H-pyrazole as an orange oil (224 mg, 75.9% after 3 steps). Crude oil was used following General Procedure 3.1 (Scheme 3.1) to afford the title compound. Flash chromatography (Hexane/EtOAc: up to 75% EtOAc) to yield a white solid (25%). MS (ESI):331.14[M+H]⁺.

¹H NMR (600 MHz, Chloroform-d) δ 7.56 (dd, J=1.9, 0.8 Hz, 1H), 7.15 (d, J=2.4 Hz, 1H), 6.93 (dd, J=3.6, 0.9 Hz, 2H), 6.91 (s, 1H), 6.83 (s, 1H), 6.54 (dd, J=3.5, 1.8 Hz, 1H), 5.59 (d, J=2.3 Hz, 1H), 3.97 (t, J=6.8 Hz, 2H), 3.85 (s, 3H), 3.44 (q, J=7.2 Hz, 2H), 1.90 (p, J=6.9 Hz, 2H), 1.60 (p, J=7.6 Hz, 3H). HR-MS (QTOF): 331.142 [M+H]⁺.

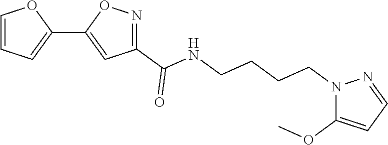 [SMILES: COc1ccnn1CCCCNC(=O)c1cc(-c2ccco2)on1]

### 5-(furan-2-yl)-N-(4-(5-methoxy-1H-pyrazol-1-yl)butyl)isoxazole-3-carboxamide

Pyrazol-3-ol was used as starting material following Scheme 3.1.1 to afford the tittle compound. General Procedure 3.1 (Scheme 3.1.1): Flash chromatography (Hexane/EtOAc: up to 75% EtOAc) to yield a white solid (12.3%).

¹H NMR (600 MHz, Chloroform-d) δ 7.56 (d, J=1.6 Hz, 1H), 7.30 (d, J=2.0 Hz, 1H), 6.93 (dd, J=3.4, 0.7 Hz, 2H), 6.92 (s, 1H), 6.83 (s, 1H), 6.54 (dd, J=3.5, 1.8 Hz, 1H), 5.48 (d, J=2.0 Hz, 1H), 3.98 (t, J=6.8 Hz, 2H), 3.86 (s, 3H), 3.44 (q, J=7.0 Hz, 3H), 1.87 (p, J=6.9 Hz, 2H), 1.60 (p, J=7.3 Hz, 2H). HR-MS (QTOF): 331.142 [M+H]⁺.

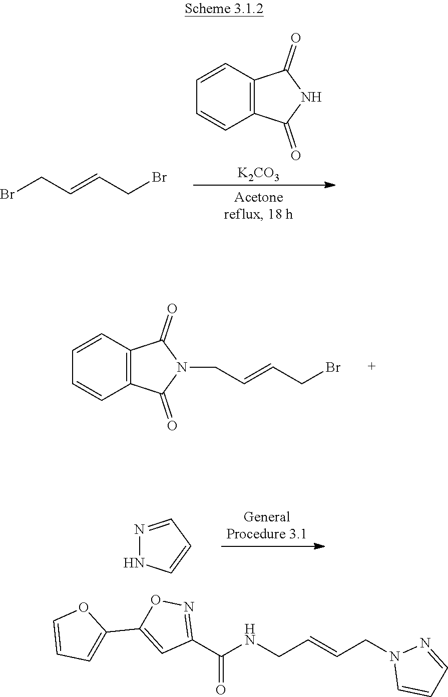

### (E)-2-(4-Bromobut-2-en-1-yl)isoindoline-1,3-dione

To a solution of phthalimide (378 mg, 2.57 mmol) in acetone (6 mL), potassium carbonate (420 mg, 3.04 mmol) was added. The mixture was stirred at room temperature for 30 minutes, then added (E)-1,4-Dibromobut-2-ene (500 mg, 2.34 mmol). The reaction was refluxed for 18 hours. After cooling to room temperature, the solvent was evaporated and the residue was dissolved in DCM. The organics were washed brine, dried over NaSO₄, filtered, and evaporated. The crude material was purified via flash column chromatography (Hexane/EtOAc=80/20) to provide the title compound (353 mg, 54% yield).

¹H NMR (400 MHz, Chloroform-d) δ 7.81 (m, 2H), 7.70 (m, 2H), 5.90 (m, 1H), 5.82 (m, 1H), 4.27 (dd, J=5.8, 1.1 Hz, 2H), 3.88 (dd, J=7.2, 0.8 Hz, 2H).

### (E)-2-(4-(1H-pyrazol-1-yl)but-2-en-1-yl)isoindoline-1,3-dione

Following General Procedure 3.1 (Scheme 3.1) to afford the tittle compound (259 mg, 77% yield). Flash column chromatography (Hexane/EtOAc=50/50).

¹H NMR (400 MHz, Chloroform-d) δ 7.85 (m, 2H), 7.72 (m, 2H), 7.49 (d, J=1.7 Hz, 1H), 7.37 (d, J=2.3 Hz, 1H), 6.25 (t, J=2.1 Hz, 1H), 5.90 (m, 1H), 5.71 (m, 1H), 4.74 (dd, J=6.0, 1.3 Hz, 2H), 4.31 (dd, J=6.1, 1.3 Hz, 2H).

### (E)-4-(1H-pyrazol-1-yl)but-2-en-1-amine

Following General Procedure 3.1 (Scheme 3.1) to afford the tittle compound. The crude material was used directly for next step without further purification.

### (E)-N-(4-(1H-pyrazol-1-yl)but-2-en-1-yl)-5-(furan-2-yl)isoxazole-3-carboxamide

Crude amine was used for amide bond formation reaction via General Procedure 1 to provide the title compound (24 mg, 42% yield of two steps) as a white solid. Flash chromatography: (Hexane/EtOAc=30/70).

¹H NMR (400 MHz, Chloroform-d) δ 7.57 (d, J=1.4 Hz, 1H), 7.52 (d, J=1.9 Hz, 1H), 7.40 (d, J=2.3 Hz, 1H), 7.00 (s, OH), 6.95 (d, J=3.5 Hz, 1H), 6.85 (s, 1H), 6.55 (m, 2H), 6.27 (t, J=2.1 Hz, 1H), 5.93 (m, 1H), 5.72 (m, 1H), 4.77 (dd, J=6.0, 1.4 Hz, 2H), 4.10 (td, J=5.9, 1.4 Hz, 2H). MS (ESI): 299.11 [M+H]⁺.

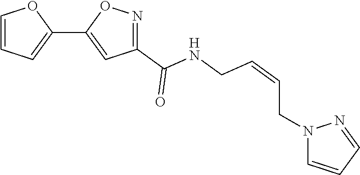 [SMILES: O=C(NC/C=C\Cn1cccn1)c1cc(-c2ccco2)on1]

### (Z)—N-(4-(1H-pyrazol-1-yl)but-2-en-1-yl)-5-(furan-2-yl)isoxazole-3-carboxamide

(Z)-1,4-dichlorobut-2-ene was used as the starting material for Gabriel amine synthesis by the same method (Scheme 3.1.2). Step 1: 82% yield; step 2: 55% yield; step 3 and 4: 40% yield of two steps, as a yellow solid. Flash chromatography: (up to 100% EtOAc).

¹H NMR (400 MHz, Chloroform-d) δ 7.62 (t, J=5.8 Hz, 1H), 7.56 (s, 2H), 7.45 (d, J=2.3 Hz, 1H), 6.94 (d, J=3.5 Hz, 1H), 6.86 (s, 1H), 6.54 (dd, J=3.5, 1.8 Hz, 1H), 6.26 (t, J=2.1 Hz, 1H), 5.85 (m, 2H), 4.90 (d, J=6.4 Hz, 2H), 4.20 (t, J=6.3 Hz, 2H). MS (ESI): 299.11 [M+H]⁺.

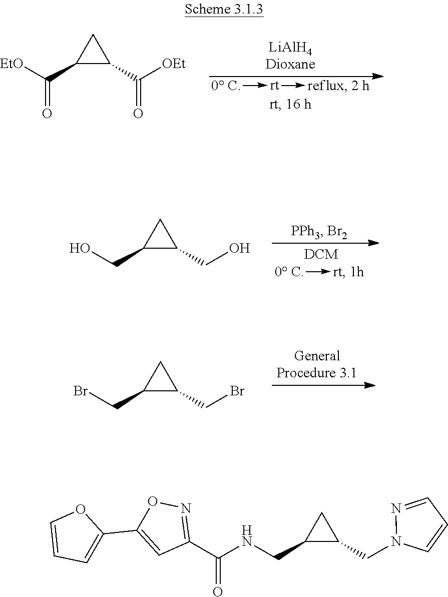

### ((1S,2S)-2-Ethylcyclopropyl)methanol

To a solution of diethyl (1S,2S)-cyclopropane-1,2-dicarboxylate (471 μL, 2.69 mmol) in dioxane (8 mL), lithium tetrahydridoaluminate (2M in THF) (2.95 mL, 5.91 mmol) was added slowly at 0° C. under N₂ balloon protection. The mixture was warmed to room temperature and stirred for 16 hours. After cooling to 0° C., the reaction was quenched with saturated ammonium chloride, diluted with EtOAc and stirred for 5 hours, during which light yellow gel was formed. The resulting mixture was filtered through a pad of celite. The celite layer was washed with EtOAc for three times. Combined organic layers were concentrated and purified via flash column chromatography (EtOAc/MeOH=90/10) to provide the title compound (204 mg, 75% yield).

¹H NMR (400 MHz, Chloroform-d) δ 3.79 (m, 2H), 3.41 (s, 1H), 3.31 (s, 1H), 3.12 (m, 2H), 1.03 (m, 2H), 0.45 (m, 2H).

### (1S,2S)-1,2-bis(Bromomethyl)cyclopropane

Bromine (317 μL, 6.15 mmol) was added slowly at 0° C. to the solution of triphenylphosphine (1612 mg, 6.15 mmol) in DCM (10 mL). The mixture was stirred for 15 mins and was added ((1S,2S)-2-ethylcyclopropyl)methanol (286 mg, 2.79 mmol). After stirring at room temperature for 1 hour, the solvent was evaporated under reduced pressure. The residue was purified via flash column chromatography (Hexane/EtOAc=90/10) to provide the title compound (291 mg, 46% yield).

¹H NMR (400 MHz, Chloroform-d) δ 3.33 (m, 4H), 1.33 (m, 2H), 0.84 (m, 2H).

### N-(((1S,2S)-2-((1H-pyrazol-1-yl)methyl)cyclopropyl)methyl)-5-(furan-2-yl)isoxazole-3-carboxamide

(1S,2S)-1,2-bis(bromomethyl)cyclopropane was used for Gabriel amine synthesis via General Procedure 3.1 (Scheme 3.1). Step 3: 23% yield; step 4: 30% yield; step 5 and 6: 48% yield of two steps, as a white solid. Flash chromatography: (100% EtOAc).

¹H NMR (400 MHz, Chloroform-d) δ 7.98 (s, 1H), 7.66 (d, J=1.9 Hz, 1H), 7.57 (d, J=1.5 Hz, 1H), 7.40 (d, J=2.2 Hz, 1H), 6.95 (dd, J=3.5, 0.7 Hz, 1H), 6.84 (s, 1H), 6.55 (dd, J=3.5, 1.8 Hz, 1H), 6.25 (t, J=2.1 Hz, 1H), 4.34 (dd, J=13.8, 5.1 Hz, 1H), 3.74 (m, 2H), 2.97 (ddd, J=13.6, 9.0, 4.2 Hz, 1H), 1.22 (m, 2H), 0.71 (dd, J=7.5, 6.2 Hz, 2H). MS (ESI): 313.13 [M+H]⁺.

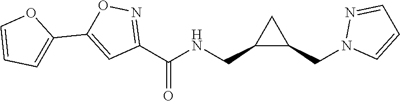 [SMILES: O=C(NC[C@H]1C[C@H]1Cn1cccn1)c1cc(-c2ccco2)on1]

### N-(((1S,2R)-2-((1H-pyrazol-1-yl)methyl)cyclopropyl)methyl)-5-(furan-2-yl)isoxazole-3-carboxamide

Diethyl (1R,2S)-cyclopropane-1,2-dicarboxylate was used as the starting material for Gabriel amine synthesis by the same method (Scheme 3.1.3). Step 1: 87% yield; step 2: 46% yield; step 3: 23% yield; step 4: 30% yield; step 5 and 6: 48% yield of two steps, as a white solid. Flash chromatography: (Hexane/EtOAc=30/70).

¹H NMR (400 MHz, Chloroform-d) δ 9.78 (d, J=9.0 Hz, 1H), 7.76 (d, J=1.9 Hz, 1H), 7.57 (dd, J=1.8, 0.7 Hz, 1H), 7.44 (d, J=2.1 Hz, 1H), 6.95 (d, J=3.5 Hz, 1H), 6.87 (s, 1H), 6.55 (dd, J=3.5, 1.8 Hz, 1H), 6.27 (t, J=2.1 Hz, 1H), 4.51 (m, 2H), 3.89 (m, 1H), 2.82 (ddd, J=14.5, 10.5, 2.7 Hz, 1H), 1.28 (m, 2H), 0.90 (td, J=8.6, 5.4 Hz, 1H), 0.26 (q, J=5.7 Hz, 1H). MS (ESI): 313.13 [M+H]⁺.

### General Procedure 3.2 Use of Purified Amide Product

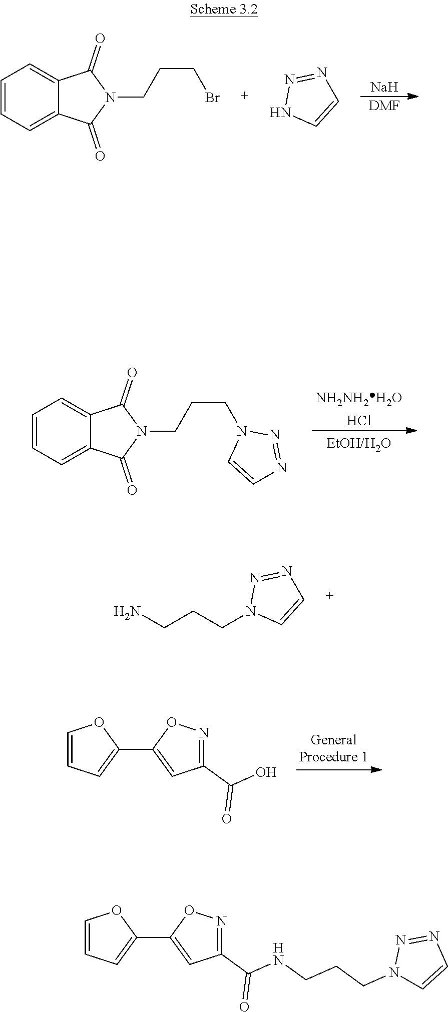

### 3-(1H-1,2,3-triazol-1-yl)propan-1-amine

To a solution of 2-(3-(1H-1,2,3-triazol-1-yl)propyl)isoindoline-1,3-dione (obtain through procedure describe above in Scheme 3.1) (350 mg, 1.37 mmol) in EtOH:DIW (3:1) mixture was added hydrazine monohydrate (60% w/w in H₂O) (150 mg, 3.00 mmol) and refluxed at 90° C. for 16 hours. To the resulting solution, 6M HCl was added to acidify to pH 2, refluxed for 2 hours and filtered through celite. Filtrate was concentrated in vacuo. Resulting residue was dissolved in DIW and washed with DCM twice. Combine aqueous was basified to pH 12 with 6M NaOH, washed with DCM twice, concentrated in vacuo and put under high vacuum for 3 hours. Resulted solid was grinded to powder and triturated with hot DCM three times to yield yellow oil (119 mg, 69.2%). Isolated amine was used for amide coupling with corresponding carboxylic acid following General Procedure 1 (Scheme 1).

¹H NMR (400 MHz, Chloroform-d) δ 7.70 (d, J=1.0 Hz, 1H), 7.56 (d, J=1.0 Hz, 1H), 4.51 (t, J=6.9 Hz, 2H), 2.72 (t, J=6.7 Hz, 2H), 2.03 (p, J=6.8 Hz, 2H).

### N-(3-(1H-1,2,3-triazol-1-yl)propyl)-5-(furan-2-yl)isoxazole-3-carboxamide

3-(1H-1,2,3-triazol-1-yl)propan-1-amine was used for amide coupling following General Procedure 1. Flash chromatography (EtOAc/MeOH: 80/20) to yield a white solid (49.2%).

¹H NMR (400 MHz, Chloroform-d) δ 7.73 (d, J=1.0 Hz, 1H), 7.69 (d, J=1.0 Hz, 1H), 7.58 (dd, J=1.8, 0.7 Hz, 1H), 7.07 (d, J=7.5 Hz, 1H), 6.96 (dd, J=3.5, 0.8 Hz, 1H), 6.85 (s, 1H), 6.56 (dd, J=3.5, 1.8 Hz, 1H), 4.51 (t, J=6.7 Hz, 2H), 3.50 (q, J=6.5 Hz, 2H), 2.27 (p, J=6.6 Hz, 2H). MS (ESI): 288.11 [M+H]⁺.

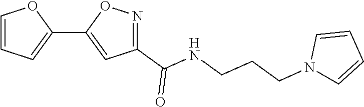 [SMILES: O=C(NCCCn1cccc1)c1cc(-c2ccco2)on1]

### N-(3-(1H-pyrrol-1-yl)propyl)-5-(furan-2-yl)isoxazole-3-carboxamide

General Procedure 3.2 (Scheme 3.2): Flash chromatography (EtOAc/MeOH=80/20). 31.2% yield as a white solid.

1H NMR (400 MHz, Chloroform-d) δ 7.58 (dd, J=1.8, 0.8 Hz, 1H), 6.95 (dd, J=3.5, 0.8 Hz, 1H), 6.85 (s, 1H), 6.76 (s, 1H), 6.68 (t, J=2.1 Hz, 2H), 6.57-6.55 (m, 1H), 6.16 (t, J=2.1 Hz, 2H), 4.00 (t, J=6.8 Hz, 2H), 3.44 (q, J=6.7 Hz, 2H), 2.11 (p, J=6.8 Hz, 2H). MS (ESI): 286.11 [M+H]⁺.

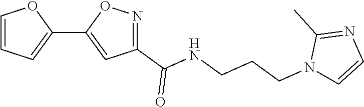 [SMILES: Cc1nccn1CCCNC(=O)c1cc(-c2ccco2)on1]

### 5-(furan-2-yl)-N-(3-(2-methyl-1H-imidazol-1-yl)propyl)isoxazole-3-carboxamide

General Procedure 3.2 (Scheme 3.2): Flash chromatography (EtOAc/MeOH=80/20). 54% yield as a white solid.

¹H NMR (400 MHz, Chloroform-d) δ 7.58 (td, J=1.6, 0.7 Hz, 1H), 6.96 (td, J=1.9, 0.8 Hz, 2H), 6.93 (s, 1H), 6.91 (d, J=1.4 Hz, 1H), 6.86 (s, 1H), 6.59-6.54 (m, 1H), 3.96 (t, J=7.2 Hz, 2H), 3.50 (q, J=7.0 Hz, 2H), 2.07 (p, J=7.0 Hz, 2H).

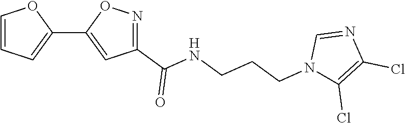 [SMILES: O=C(NCCCn1cnc(Cl)c1Cl)c1cc(-c2ccco2)on1]

### N-(3-(4,5-dichloro-1H-imidazol-1-yl)propyl)-5-(furan-2-yl)isoxazole-3-carboxamide

General Procedure 3.2 (Scheme 3.2): Flash chromatography (Hexane/EtOAc: up to 100% EtOAc). 30.5% yield as a white solid.

¹H NMR (400 MHz, Chloroform-d) δ 7.58 (dd, J=1.8, 0.6 Hz, 1H), 7.54 (s, 1H), 7.03 (s, 1H), 6.96 (dd, J=3.5, 0.7 Hz, 1H), 6.86 (s, 1H), 6.58-6.54 (m, 1H), 4.04 (t, J=7.0 Hz, 2H), 3.51 (q, J=6.5 Hz, 2H), 2.12 (p, J=6.8 Hz, 2H).

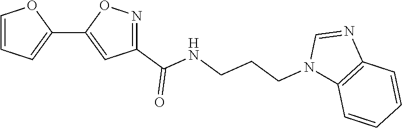 [SMILES: O=C(NCCCn1cnc2ccccc21)c1cc(-c2ccco2)on1]

### N-(3-(1H-benzo[d]imidazol-1-yl)propyl)-5-(furan-2-yl)isoxazole-3-carboxamide

General Procedure 3.2 (Scheme 3.2): Flash chromatography (EtOAc/MeOH=80/20). 35.3% yield as a white solid.

¹H NMR (400 MHz, Chloroform-d) δ 8.01 (s, 1H), 7.82-7.77 (m, 1H), 7.56 (dd, J=1.8, 0.7 Hz, 1H), 7.43-7.37 (m, 1H), 7.32-7.25 (m, 2H), 7.08 (s, 1H), 6.93 (dd, J=3.5, 0.8 Hz, 1H), 6.84 (s, 1H), 6.54 (ddd, J=3.5, 1.8, 0.6 Hz, 1H), 4.28 (t, J=7.0 Hz, 2H), 3.49 (q, J=6.6 Hz, 2H), 2.22 (p, J=6.9 Hz, 2H). MS (ESI): 337.13 [M+H]⁺.

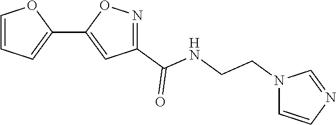 [SMILES: O=C(NCCn1ccnc1)c1cc(-c2ccco2)on1]

### N-(2-(1H-imidazol-1-yl)ethyl)-5-(furan-2-yl)isoxazole-3-carboxamide

General Procedure 3.2 (Scheme 3.2): Flash chromatography (EtOAc/MeOH=80/20). 12.9% yield as a white solid.

¹H NMR (600 MHz, Chloroform-d) δ 7.78 (s, 1H), 7.57 (dd, J=2.0, 0.8 Hz, 1H), 7.13 (s, 1H), 6.99 (s, 1H), 6.94 (d, J=3.5 Hz, 1H), 6.83 (s, 1H), 6.56-6.53 (m, 1H), 4.28 (t, J=5.5 Hz, 2H), 3.81 (q, J=6.1 Hz, 2H). MS (ESI): 273.10 [M+H]⁺.

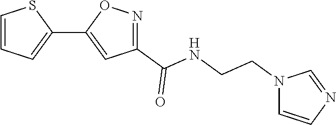 [SMILES: O=C(NCCn1ccnc1)c1cc(-c2cccs2)on1]

### N-(2-(1H-imidazol-1-yl)ethyl)-5-(thiophen-2-yl)isoxazole-3-carboxamide

General Procedure 3.2 (Scheme 3.2): Flash chromatography (EtOAc/MeOH=80/20). 14.2% yield as a yellow solid.

¹H NMR (600 MHz, Chloroform-d) δ 7.71 (s, 1H), 7.53 (dd, J=3.7, 1.1 Hz, 1H), 7.49 (dd, J=5.0, 1.1 Hz, 1H), 7.19 (s, 1H), 7.14 (dd, J=5.0, 3.7 Hz, 2H), 6.99 (s, 1H), 6.79 (s, 1H), 4.25 (t, J=6.3 Hz, 2H), 3.80 (q, J=6.2 Hz, 2H). MS (ESI): 289.07 [M+H]⁺.

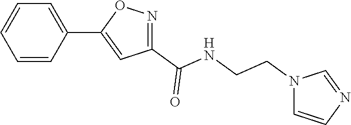 [SMILES: O=C(NCCn1ccnc1)c1cc(-c2ccccc2)on1]

### N-(2-(1H-imidazol-1-yl)ethyl)-5-phenylisoxazole-3-carboxamide

General Procedure 3.2 (Scheme 3.2): Flash chromatography (EtOAc/MeOH=80/20). 31.2% yield as a white solid.

¹H NMR (400 MHz, Chloroform-d) δ 7.81-7.76 (m, 2H), 7.57 (s, 1H), 7.51-7.47 (m, 3H), 7.16 (t, 1H), 7.11 (s, 1H), 6.98 (s, 1H), 6.95 (s, 1H), 4.24 (t, J=5.9 Hz, 2H), 3.80 (q, J=6.2 Hz, 2H). MS (ESI): 283.12 [M+H]⁺.

### General Procedure 4: Synthesis of Two Carbon Linker Analogs

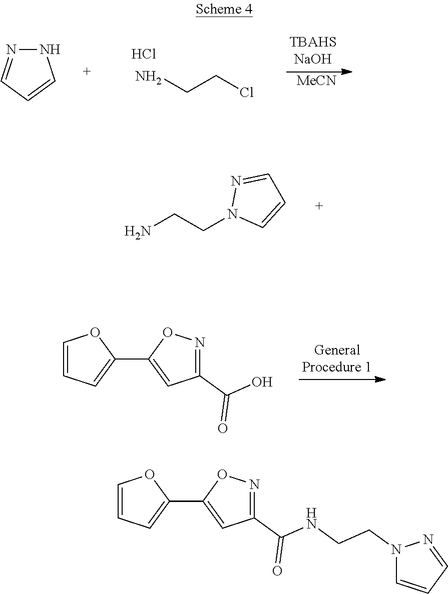

### N-(2-(1H-pyrazol-1-yl)ethyl)-5-(furan-2-yl)isoxazole-3-carboxamide

To a solution of pyrazole (250 mg, 3.67 mmol) in MeCN was added sodium hydroxide (734 mg, 18.4 mmol) powder and tetrabutylammonium hydrogen sulfate (TBAS) (62 mg, 0.184 mmol). After stirring at room temperature for 30 minutes, 2-chloroethylamine hydrochloride (511 mg, 4.41 mmol) was added. The reaction was refluxed for 18 hours before being cooled to room temperature and filtered through celite. Filtrate was concentrated in vacuo obtain 2-(1H-pyrazol-1-yl)ethan-1-amine (450 mg, quant.) crude as yellow oil. The crude was used for subsequent amide coupling (Scheme 1) without purification. Flash chromatography (EtOAc/MeOH=90/10) to yield a white solid (23.8%).

¹H NMR (600 MHz, Chloroform-d) δ 7.57 (dd, J=1.9, 0.9 Hz, 1H), 7.56 (dd, J=1.7, 0.8 Hz, 1H), 7.40 (d, J=1.6 Hz, 1H), 7.33 (s, 1H), 6.93 (dd, J=3.5, 0.8 Hz, 1H), 6.83 (s, 1H), 6.54 (dd, J=3.5, 1.8 Hz, 1H), 6.26 (t, J=2.1 Hz, 1H), 4.37 (t, J=5.5 Hz, 2H), 3.91 (q, J=5.9 Hz, 2H). MS (ESI): 273.10 [M+H]⁺.

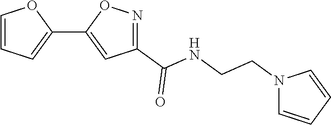 [SMILES: O=C(NCCn1cccc1)c1cc(-c2ccco2)on1]

### N-(2-(1H-pyrrol-1-yl)ethyl)-5-(furan-2-yl)isoxazole-3-carboxamide

General Procedure 4 (Scheme 4): Flash chromatography (Hexane/EtOAc: up to 75% EtOAc). 23.8% yield as a white solid.

¹H NMR (400 MHz, Chloroform-d) δ 7.57 (dd, J=1.8, 0.8 Hz, 1H), 6.94 (dd, J=3.5, 0.8 Hz, 1H), 6.85 (s, 1H), 6.68 (t, J=2.1 Hz, 2H), 6.56-6.54 (m, 1H), 6.20-6.16 (m, 2H), 4.13 (t, J=5.4 Hz, 2H), 3.76 (q, J=5.5 Hz, 2H). MS (ESI): 270.01 [M+H]⁺.

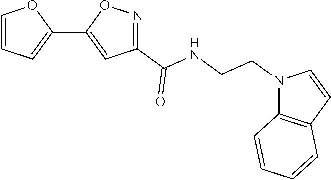 [SMILES: O=C(NCCn1ccc2ccccc21)c1cc(-c2ccco2)on1]

### N-(2-(1H-indol-1-yl)ethyl)-5-(furan-2-yl)isoxazole-3-carboxamide

General Procedure 4 (Scheme 4): Flash chromatography (Hexane/EtOAc: up to 75% EtOAc). 20% yield as a yellow solid.

¹H NMR (400 MHz, Chloroform-d) δ 7.63 (ddt, J=7.9, 1.2, 0.6 Hz, 1H), 7.56 (dt, J=1.8, 0.6 Hz, 1H), 7.38 (dq, J=8.2, 0.9 Hz, 1H), 7.20 (dddd, J=8.2, 7.0, 1.1, 0.5 Hz, 1H), 7.13-7.09 (m, 1H), 7.09-7.07 (m, 1H), 6.95-6.89 (m, 2H), 6.84 (d, J=0.6 Hz, 1H), 6.54 (dd, J=3.5, 0.5 Hz, 1H), 6.51 (dt, J=3.2, 0.7 Hz, 1H), 4.38 (t, J=6.2 Hz, 2H), 3.82 (q, J=6.3 Hz, 2H). MS (ESI): 322.12 [M+H]⁺.

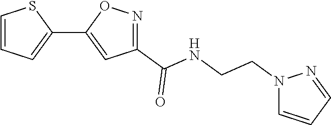 [SMILES: O=C(NCCn1cccn1)c1cc(-c2cccs2)on1]

### N-(2-(1H-pyrazol-1-yl)ethyl)-5-(thiophen-2-yl)isoxazole-3-carboxamide

General Procedure 4 (Scheme 4): Flash chromatography (Hexane/EtOAc: up to 100% EtOAc). 9.1% yield as a yellow solid.

¹H NMR (400 MHz, Chloroform-d) δ 7.57 (dt, J=1.6, 0.7 Hz, 1H), 7.52 (dt, J=3.7, 1.0 Hz, 1H), 7.47 (dt, J=5.0, 1.0 Hz, 1H), 7.39 (dd, J=2.3, 0.7 Hz, 1H), 7.12 (ddd, J=5.1, 3.7, 0.9 Hz, 1H), 6.79 (d, J=0.8 Hz, 1H), 6.25 (ddd, J=2.7, 2.0, 0.8 Hz, 1H), 4.39-4.32 (m, 2H), 3.90 (q, J=5.7 Hz, 2H). MS (ESI): 289.07 [M+H]⁺.

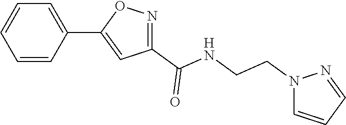 [SMILES: O=C(NCCn1cccn1)c1cc(-c2ccccc2)on1]

### N-(2-(1H-pyrazol-1-yl)ethyl)-5-phenylisoxazole-3-carboxamide

General Procedure 4 (Scheme 4): Flash chromatography (Hexane/EtOAc: up to 100% EtOAc). 23.9% yield as an off-white solid.

¹H NMR (400 MHz, Chloroform-d) δ 7.79-7.74 (m, 2H), 7.58 (dd, J=2.0, 0.7 Hz, 1H), 7.50-7.43 (m, 3H), 7.40 (dd, J=2.3, 0.7 Hz, 1H), 7.37 (s, 1H), 6.94 (s, 1H), 6.26 (t, J=2.1 Hz, 1H), 4.37 (t, J=5.6 Hz, 2H), 3.92 (q, J=5.7 Hz, 2H). MS (ESI): 283.12 [M+H]⁺.

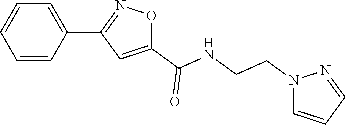 [SMILES: O=C(NCCn1cccn1)c1cc(-c2ccccc2)no1]

### N-(2-(1H-pyrazol-1-yl)ethyl)-3-phenylisoxazole-5-carboxamide

General Procedure 4 (Scheme 4): Flash chromatography (Hexane/EtOAc: up to 100% EtOAc). 21% yield as a white solid.

¹H NMR (600 MHz, Chloroform-d) δ 7.85-7.79 (m, 2H), 7.59 (d, J=1.5 Hz, 1H), 7.50-7.44 (m, 4H), 7.42-7.37 (m, 2H), 7.25 (s, 1H), 6.28 (t, J=2.1 Hz, 1H), 4.37 (t, J=5.6 Hz, 2H), 3.94 (q, J=5.7 Hz, 2H). MS (ESI): 283.11 [M+H]⁺.

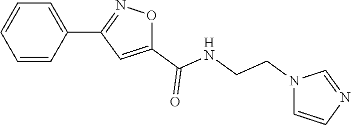 [SMILES: O=C(NCCn1ccnc1)c1cc(-c2ccccc2)no1]

### N-(2-(1H-imidazol-1-yl)ethyl)-3-phenylisoxazole-5-carboxamide

General Procedure 4 (Scheme 4): Flash chromatography (EtOAc/MeOH=90/10). 20.2% yield as a white solid.

¹H NMR (400 MHz, d-DMSO) δ 9.11 (t, J=5.7 Hz, 1H), 7.91-7.85 (m, 2H), 7.61 (s, 1H), 7.58 (s, 1H), 7.52-7.47 (m, 3H), 7.16 (s, 1H), 6.86 (s, 1H), 4.15 (t, J=5.8 Hz, 2H), 3.58 (q, J=5.5 Hz, 2H). MS (ESI): 283.11 [M+H]⁺.

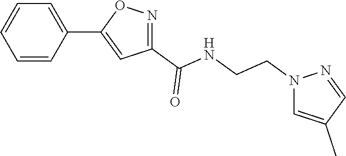 [SMILES: Cc1cnn(CCNC(=O)c2cc(-c3ccccc3)on2)c1]

### 5-(furan-2-yl)-N-(2-(4-methyl-1H-pyrazol-1-yl)ethyl)isoxazole-3-carboxamide

General Procedure 4 (Scheme 4): Flash chromatography (Hexane/EtOAc: up to 100% EtOAc). 34.4% yield as a yellow solid.

¹H NMR (400 MHz, Chloroform-d) δ 7.57 (dq, J=1.9, 0.8 Hz, 1H), 7.39 (s, 1H), 7.36 (s, 1H), 7.17 (s, 1H), 6.94 (d, J=3.4 Hz, 1H), 6.84 (s, 1H), 6.55 (dd, J=3.5, 0.6 Hz, 1H), 4.29 (t, J=6.1 Hz, 2H), 3.88 (q, J=5.5 Hz, 2H), 2.06 (s, 3H). MS (ESI): 287.11 [M+H]⁺.

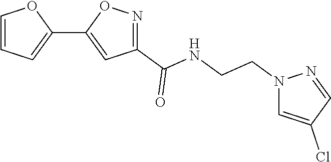 [SMILES: O=C(NCCn1cc(Cl)cn1)c1cc(-c2ccco2)on1]

### N-(2-(4-chloro-1H-pyrazol-1-yl)ethyl)-5-(furan-2-yl)isoxazole-3-carboxamide

General Procedure 4 (Scheme 4): Flash chromatography (Hexane/EtOAc: up to 75% EtOAc). 40.2% yield as a white solid.

¹H NMR (400 MHz, Chloroform-d) δ 7.56 (dt, J=1.8, 0.8 Hz, 1H), 7.47 (s, 1H), 7.38 (s, 1H), 7.26 (s, 1H), 6.94 (d, J=3.5 Hz, 1H), 6.83 (s, 1H), 6.54 (ddd, J=3.5, 1.8, 0.8 Hz, 1H), 4.29 (t, J=5.6 Hz, 2H), 3.89 (q, J=6.3 Hz, 2H). MS (ESI): 305.04 [M+H]⁺.

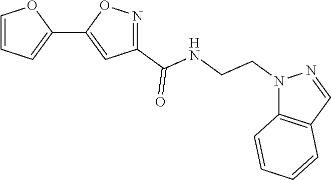 [SMILES: O=C(NCCn1ncc2ccccc21)c1cc(-c2ccco2)on1]

### N-(2-(1H-indazol-1-yl)ethyl)-5-(furan-2-yl)isoxazole-3-carboxamide

General Procedure 4 (Scheme 4): Flash chromatography (Hexane/EtOAc: up to 100% EtOAc) to yield a white solid (32.7%, 64.6% regioselective).

¹H NMR (400 MHz, Chloroform-d) δ 8.04 (d, J=1.0 Hz, 1H), 7.72 (dt, J=8.1, 1.0 Hz, 1H), 7.55 (dd, J=1.8, 0.8 Hz, 1H), 7.42 (dq, J=8.5, 1.0 Hz, 1H), 7.38-7.34 (m, 1H), 7.30 (s, 1H), 7.13 (ddd, J=7.9, 6.7, 1.0 Hz, 1H), 6.91 (dd, J=3.5, 0.8 Hz, 1H), 6.82 (s, 1H), 6.53 (dd, J=3.5, 1.8 Hz, 1H), 4.61 (t, J=5.7 Hz, 3H), 3.98 (q, J=6.0 Hz, 3H).

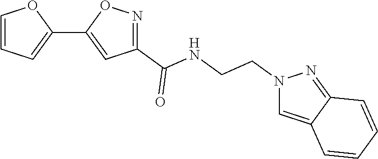 [SMILES: O=C(NCCn1cc2ccccc2n1)c1cc(-c2ccco2)on1]

### N-(2-(2H-indazol-2-yl)ethyl)-5-(furan-2-yl)isoxazole-3-carboxamide

General Procedure 4 (Scheme 4): Flash chromatography (Hexane/EtOAc: up to 100% EtOAc) to yield a white solid (32.7%, 35.4% regioselective).

¹H NMR (400 MHz, Chloroform-d) δ 7.94 (d, J=1.0 Hz, 1H), 7.72 (dt, J=8.8, 1.0 Hz, 1H), 7.64 (dt, J=8.4, 1.1 Hz, 1H), 7.55 (dd, J=1.8, 0.7 Hz, 1H), 7.38 (s, 1H), 7.34-7.27 (m, 1H), 7.12-7.06 (m, 1H), 6.93 (dd, J=3.5, 0.7 Hz, 1H), 6.84 (s, 1H), 6.54 (dd, J=3.6, 1.8 Hz, 1H), 4.67 (t, J=5.6 Hz, 2H), 4.06 (q, J=5.9 Hz, 2H).

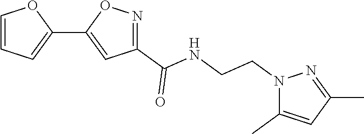 [SMILES: Cc1cc(C)n(CCNC(=O)c2cc(-c3ccco3)on2)n1]

### N-(2-(3,5-dimethyl-1H-pyrazol-1-yl)ethyl)-5-(furan-2-yl)isoxazole-3-carboxamide

General Procedure 4 (Scheme 4): Flash chromatography (Hexane/EtOAc: up to 100% EtOAc). 13.7% yield as a light pink solid.

¹H NMR (400 MHz, Chloroform-d) δ 7.55 (dd, J=1.8, 0.7 Hz, 1H), 7.43 (s, 1H), 6.93 (dd, J=3.5, 0.7 Hz, 1H), 6.83 (s, 1H), 6.54 (ddd, J=3.6, 1.8, 0.5 Hz, 1H), 5.80 (s, 1H), 4.18 (t, J=4.9 Hz, 2H), 3.85 (q, J=5.8 Hz, 2H), 2.23 (s, 3H), 2.20 (2, J=0.7 Hz, 3H).

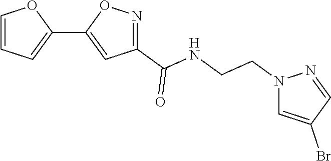 [SMILES: O=C(NCCn1cc(Br)cn1)c1cc(-c2ccco2)on1]

### N-(2-(4-bromo-1H-pyrazol-1-yl)ethyl)-5-(furan-2-yl)isoxazole-3-carboxamide

General Procedure 4 (Scheme 4): Flash chromatography (Hexane/EtOAc: up to 75% EtOAc). 27.5% yield as a white solid.

¹H NMR (400 MHz, Chloroform-d) δ 7.56 (dd, J=1.8, 0.8 Hz, 1H), 7.51 (s, 1H), 7.41 (s, 1H), 7.26 (s, 1H), 6.94 (d, J=3.5 Hz, 1H), 6.83 (s, 1H), 6.54 (dd, J=3.5, 1.8 Hz, 1H), 4.32 (t, J=5.6 Hz, 2H), 3.89 (q, J=5.9 Hz, 2H). MS (ESI): 351.01 [M+H]⁺.

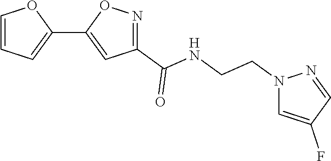 [SMILES: O=C(NCCn1cc(F)cn1)c1cc(-c2ccco2)on1]

### N-(2-(4-fluoro-1H-pyrazol-1-yl)ethyl)-5-(furan-2-yl)isoxazole-3-carboxamide

General Procedure 4 (Scheme 4): Flash chromatography (Hexane/EtOAc: up to 75% EtOAc). 21.5% yield as a white solid.

¹H NMR (400 MHz, Chloroform-d) δ 7.56 (dd, J=1.8, 0.8 Hz, 1H), 7.38 (dd, J=4.2, 0.8 Hz, 1H), 7.28 (dd, J=4.8, 0.8 Hz, 1H), 6.93 (d, J=3.5 Hz, 1H), 6.83 (s, 1H), 6.54 (dd, J=3.5, 1.8 Hz, 1H), 4.24 (t, J=5.8 Hz, 2H), 3.88 (q, J=5.9 Hz, 2H).

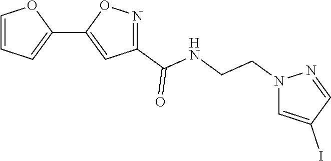 [SMILES: O=C(NCCn1cc(I)cn1)c1cc(-c2ccco2)on1]

### 5-(furan-2-yl)-N-(2-(4-iodo-1H-pyrazol-1-yl)ethyl)isoxazole-3-carboxamide

General Procedure 4 (Scheme 4): Flash chromatography (Hexane/EtOAc: up to 75% EtOAc). 21.7% yield as a yellow solid.

¹H NMR (400 MHz, Chloroform-d) δ 7.56 (ddd, J=2.9, 1.1, 0.7 Hz, 2H), 7.44 (q, J=0.6 Hz, 1H), 7.27 (d, J=5.6 Hz, 1H), 6.94 (dt, J=3.5, 1.0 Hz, 1H), 6.83 (d, J=1.0 Hz, 1H), 6.57-6.51 (m, 1H), 4.35 (t, J=5.1 Hz, 2H), 3.88 (q, J=5.7 Hz, 2H).

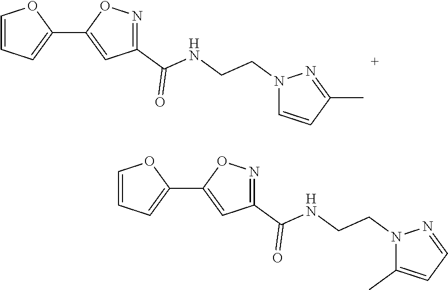 [SMILES: Cc1ccn(CCNC(=O)c2cc(-c3ccco3)on2)n1 ; Cc1ccnn1CCNC(=O)c1cc(-c2ccco2)on1]

### 5-(furan-2-yl)-N-(2-(3-methyl-1H-pyrazol-1-yl)ethyl)isoxazole-3-carboxamide & 5-(furan-2-yl)-N-(2-(5-methyl-1H-pyrazol-1-yl)ethyl)isoxazole-3-carboxamide (10:7 ratio)

General Procedure 4 (Scheme 4): 5-methyl-1H-pyrazole was used as starting material. Flash chromatography (Hexane/EtOAc: up to 75% EtOAc). 47.9% yield mixture as a white solid.

3-methyl: ¹H NMR (400 MHz, Chloroform-d) δ 7.55 (dt, J=1.8, 0.9 Hz, 1H), 7.48-7.40 (m, 2H), 6.93 (dt, J=3.5, 0.9 Hz, 1H), 6.83 (d, J=2.3 Hz, 1H), 6.54 (ddt, J=3.5, 1.8, 0.8 Hz, 1H), 6.03-6.00 (m, 1H), 4.30-4.22 (m, 2H), 3.93-3.83 (m, 2H), 2.26 (t, J=0.6 Hz, 3H).

5-methyl: ¹H NMR (400 MHz, Chloroform-d) δ 7.55 (dt, J=1.8, 0.9 Hz, 1H), 7.33 (s, 1H), 7.26 (dq, J=2.0, 0.5 Hz, 1H), 6.93 (dt, J=3.5, 0.9 Hz, 1H), 6.83 (s, 1H), 6.54 (ddt, J=3.5, 1.8, 0.8 Hz, 1H), 6.03-6.00 (m, 1H), 4.29-4.23 (m, 2H), 3.91-3.83 (m, 2H), 2.29 (q, J=0.5 Hz, 3H).

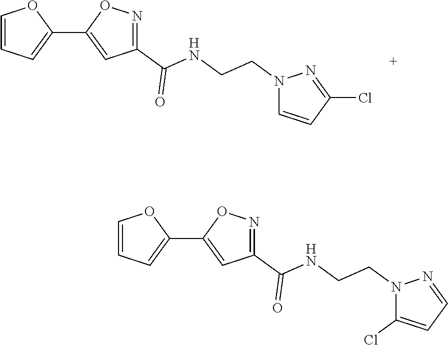 [SMILES: O=C(NCCn1ccc(Cl)n1)c1cc(-c2ccco2)on1 ; O=C(NCCn1nccc1Cl)c1cc(-c2ccco2)on1]

### N-(2-(3-chloro-1H-pyrazol-1-yl)ethyl)-5-(furan-2-yl)isoxazole-3-carboxamide+N-(2-(5-chloro-1H-pyrazol-1-yl)ethyl)-5-(furan-2-yl)isoxazole-3-carboxamide (2:1 ratio)

General Procedure 4 (Scheme 4): 5-chloro-1H-pyrazole was used as starting material. Flash chromatography (Hexane/EtOAc: up to 100% EtOAc). 18.6% yield mixture as a yellow solid.

3-chloro: ¹H NMR (400 MHz, Chloroform-d) δ 7.54 (tq, J=1.9, 0.9 Hz, 1H), 7.30 (dd, J=2.3, 0.6 Hz, 1H), 7.20 (s, 1H), 6.92 (tt, J=3.5, 0.8 Hz, 2H), 6.82 (dd, J=1.7, 0.5 Hz, 1H), 6.52 (dtd, J=3.7, 1.9, 0.6 Hz, 1H), 6.14 (dd, J=2.3, 0.6 Hz, 1H), 4.27 (dd, J=6.5, 4.9 Hz, 2H), 4.09 (qd, J=7.1, 0.6 Hz, 2H).

5-chloro: ¹H NMR (400 MHz, Chloroform-d) δ 7.54 (tq, J=1.9, 0.9 Hz, 1H), 7.50 (dd, J=1.9, 0.6 Hz, 1H), 7.40-7.33 (m, 1H), 6.92 (tt, J=3.5, 0.8 Hz, 1H), 6.82 (dd, J=1.7, 0.5 Hz, 1H), 6.52 (dtd, J=3.7, 1.9, 0.6 Hz, 1H), 6.19 (dd, J=2.0, 0.6 Hz, 1H), 4.38-4.32 (m, 2H), 4.09 (qd, J=7.1, 0.6 Hz, 2H).

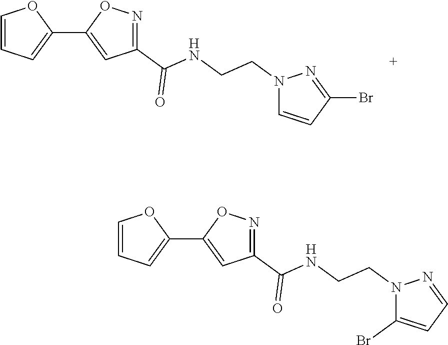 [SMILES: O=C(NCCn1ccc(Br)n1)c1cc(-c2ccco2)on1 ; O=C(NCCn1nccc1Br)c1cc(-c2ccco2)on1]

### N-(2-(3-bromo-1H-pyrazol-1-yl)ethyl)-5-(furan-2-yl)isoxazole-3-carboxamide+N-(2-(5-bromo-1H-pyrazol-1-yl)ethyl)-5-(furan-2-yl)isoxazole-3-carboxamide (2:1 ratio)

General Procedure 4 (Scheme 4): 5-bromo-1H-pyrazole was used as starting material. Flash chromatography (Hexane/EtOAc: up to 100% EtOAc). 18.2% yield mixture as a pink solid.

3-bromo: ¹H NMR (400 MHz, Chloroform-d) δ 7.57 (td, J=1.9, 0.9 Hz, 1H), 7.29 (d, J=2.2 Hz, 1H), 7.12 (s, 1H), 6.95 (t, J=3.8 Hz, 1H), 6.85 (s, 1H), 6.58-6.53 (m, 1H), 6.27 (dd, J=2.3, 0.7 Hz, 1H), 4.33 (dd, J=6.2, 5.2 Hz, 2H), 3.91 (q, J=5.7 Hz, 2H).

5-bromo: ¹H NMR (400 MHz, Chloroform-d) δ 7.57 (td, J=1.9, 0.9 Hz, 31), 7.56 (d, J=1.9 Hz, 1H), 7.32 (s, 1H), 6.95 (s, 1H), 6.85 (d, J=1.7 Hz, 3H), 6.59-6.53 (m, 1H), 6.31 (d, J=1.9 Hz, 1H), 4.43-4.38 (m, 2H), 3.91 (p, J=5.7 Hz, 2H).

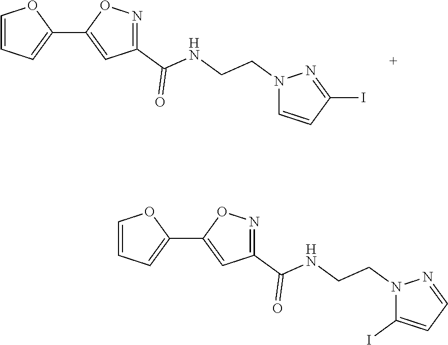 [SMILES: O=C(NCCn1ccc(I)n1)c1cc(-c2ccco2)on1 ; O=C(NCCn1nccc1I)c1cc(-c2ccco2)on1]

### N-(2-(3-iodo-1H-pyrazol-1-yl)ethyl)-5-(furan-2-yl)isoxazole-3-carboxamide+N-(2-(5-iodo-1H-pyrazol-1-yl)ethyl)-5-(furan-2-yl)isoxazole-3-carboxamide (5:3 ratio)

General Procedure 4 (Scheme 4): 5-iodo-1H-pyrazole was used as starting material. Flash chromatography (Hexane/EtOAc: up to 100% EtOAc). 16.8% yield mixture as a pink solid.

3-iodo: ¹H NMR (400 MHz, Chloroform-d) δ 7.57 (tt, J=2.1, 1.0 Hz, 1H), 7.23 (d, J=2.2 Hz, 1H), 7.13 (s, 1H), 6.96-6.95 (m, 1H), 6.85 (s, 1H), 6.56 (dq, J=3.9, 1.6 Hz, 1H), 6.42 (d, J=1.6 Hz, 1H), 4.40-4.34 (m, 2H), 3.97-3.86 (m, 2H).

5-iodo: ¹H NMR (400 MHz, Chloroform-d) δ 7.57 (m, 2H), 7.33 (s, 1H), 6.97-6.94 (m, 3H), 6.85 (s, 1H), 6.56 (dq, J=3.9, 1.6 Hz, 3H), 6.45 (d, J=1.9 Hz, 1H), 4.44 (t, 2H), 3.96-3.87 (m, 2H).

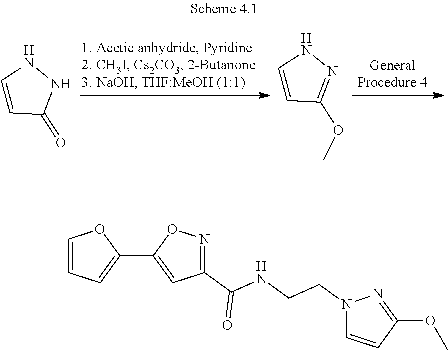

### 5-(furan-2-yl)-N-(2-(3-methoxy-1H-pyrazol-1-yl)ethyl)isoxazole-3-carboxamide

Pyrazol-3-ol was used as starting material following Scheme 4.1 to afford tittle compound. Flash chromatography (Hexane/EtOAc: up to 75% EtOAc) to yield a white solid (40.6%, 73% regioselective).

¹H NMR (500 MHz, Chloroform-d) δ 7.68 (s, 1H), 7.57 (d, J=2.0 Hz, 1H), 7.19 (d, J=2.3 Hz, 1H), 6.94 (d, J=3.2 Hz, 1H), 6.85 (s, 1H), 6.55 (dd, J=3.5, 1.9 Hz, 1H), 5.64 (d, J=2.4 Hz, 1H), 4.17 (t, J=5.8 Hz, 3H), 3.93 (s, 3H), 3.85 (q, J=5.7 Hz, 3H). MS (ESI): 303.11 [M+H]⁺.

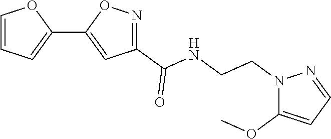 [SMILES: COc1ccnn1CCNC(=O)c1cc(-c2ccco2)on1]

### 5-(furan-2-yl)-N-(2-(5-methoxy-1H-pyrazol-1-yl)ethyl)isoxazole-3-carboxamide

Pyrazol-3-ol was used as starting material following Scheme 4.1 to afford tittle compound. Flash chromatography (Hexane/EtOAc: up to 75% EtOAc) to yield a white solid (40.6%, 37% regioselective).

¹H NMR (600 MHz, Chloroform-d) δ 7.55 (d, J=1.9 Hz, 1H), 7.44 (s, 1H), 7.34 (d, J=2.0 Hz, 1H), 6.92 (d, J=3.6 Hz, 1H), 6.83 (s, 1H), 6.54 (dd, J=3.4, 1.8 Hz, 1H), 5.50 (d, J=2.0 Hz, 1H), 4.19-4.16 (m, 3H), 3.84 (s, 3H), 3.81 (q, J=5.7 Hz, 2H). MS (ESI): 303.11 [M+H]⁺.

### General Procedure 5: Ring Closure

### General Procedure 5.1: Formation of Isoxazole Ring

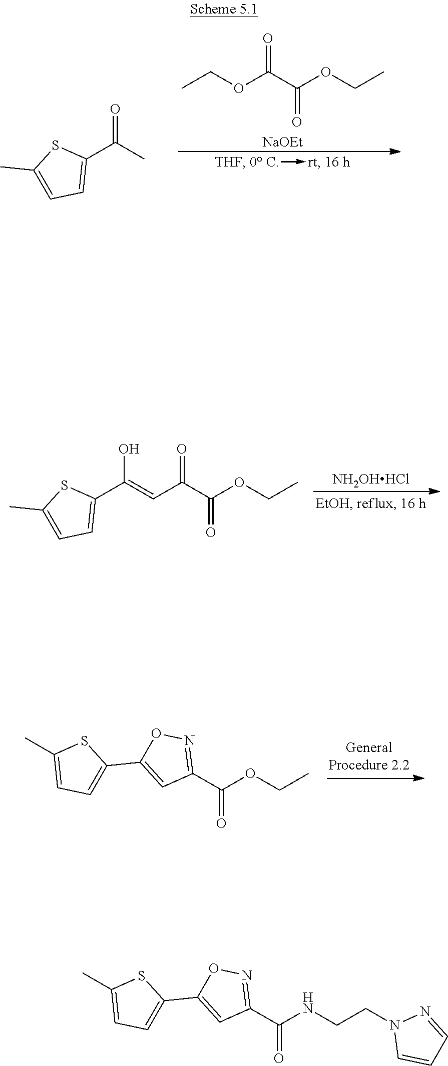

### Ethyl 4-hydroxy-4-(5-methylthiophen-2-yl)-2-oxobut-3-enoate

To a solution of 2-acetyl-5-methylthiophene (134 μL, 1.07 mmol) and diethyl oxalate (189 μL, 1.39 mmol) in THF (3 mL), sodium ethoxide (146 mg, 2.14 mmol) was added portion-wise at 0° C. The resulting mixture was stirred at room temperature for 16 hours. The solvent was evaporated and the residue was dissolved in DCM. After acidifying with 10% HCl in water to pH 3-4, the mixture was washed with brine, dried over NaSO₄, filtered, and evaporated. The crude material was purified via flash column chromatography (Hexane/EtOAc=95/5) to provide the title compound (107 mg, 42% yield).

¹H NMR (600 MHz, Chloroform-d) δ 7.65 (d, J=3.8 Hz, 1H), 6.83 (m, 2H), 4.36 (q, J=7.1 Hz, 2H), 2.55 (s, 3H), 1.37 (t, J=7.1 Hz, 3H).

### Ethyl 5-(5-methylthiophen-2-yl)isoxazole-3-carboxylate

To a solution of ethyl 4-hydroxy-4-(5-methylthiophen-2-yl)-2-oxobut-3-enoate (107 mg, 0.44 mmol) in ethanol (1.5 mL), hydroxylamine hydrochloride (34 mg, 0.49 mmol) was added. The mixture was refluxed for 16 hours. After cooling to room temperature, the solvent was evaporated under reduced pressure and the residue was dissolved in DCM. The organics were washed with brine, dried over NaSO₄, filtered, and evaporated. The crude material was purified via flash column chromatography (Hexane/EtOAc =80/20) to provide the title compound (61 mg, 58% yield), which was used for amide bond formation reaction via General Procedure 2.2 (Scheme 2.2).

¹H NMR (600 MHz, Chloroform-d) δ 7.35 (d, J=3.6 Hz, 1H), 6.79 (dt, J=3.7, 0.9 Hz, 1H), 6.67 (s, 1H), 4.45 (q, J=7.2 Hz, 2H), 2.53 (s, 3H), 1.42 (t, J=7.1 Hz, 3H).

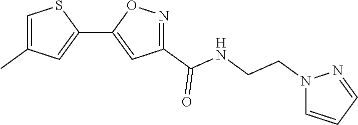 [SMILES: Cc1csc(-c2cc(C(=O)NCCn3cccn3)no2)c1]

### N-(2-(1H-pyrazol-1-yl)ethyl)-5-(4-methylthiophen-2-yl)isoxazole-3-carboxamide

General Procedure 5.1 (Scheme 5.1): step 1: 70% yield; step 2: 68% yield; step 3: 76% yield, as a pale-yellow solid. Flash chromatography: (Hexane/EtOAc=30/70).

¹H NMR (400 MHz, Chloroform-d) δ 7.58 (d, J=1.9 Hz, 1H), 7.41 (d, J=2.1 Hz, 1H), 7.36 (s, 1H), 7.33 (s, 1H), 7.06 (s, 1H), 6.76 (s, 1H), 6.27 (t, J=2.1 Hz, 1H), 4.37 (t, J=5.2 Hz, 2H), 3.91 (q, J=5.8 Hz, 2H), 2.31 (s, 3H). MS (ESI): 303.09 [M+H]⁺.

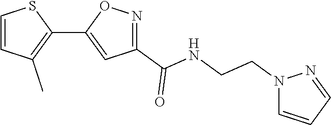 [SMILES: Cc1ccsc1-c1cc(C(=O)NCCn2cccn2)no1]

### N-(2-(1H-pyrazol-1-yl)ethyl)-5-(3-methylthiophen-2-yl)isoxazole-3-carboxamide

General Procedure 5.1 (Scheme 5.1): step 1: 40% yield; step 2: 66% yield; step 3: 43% yield, as a white solid. Flash chromatography: (100% EtOAc).

¹H NMR (600 MHz, Chloroform-d) δ 7.58 (d, J=1.5 Hz, 1H), 7.41 (d, J=2.4 Hz, 1H), 7.39 (s, 1H), 7.37 (d, J=5.0 Hz, 1H), 6.95 (d, J=5.0 Hz, 1H), 6.76 (s, 1H), 6.27 (t, J=2.2 Hz, 1H), 4.37 (t, J=5.5 Hz, 2H), 3.92 (q, J=5.8 Hz, 1H), 2.46 (s, 4H). MS (ESI): 303.09 [M+H]⁺.

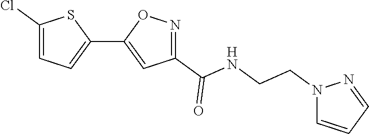 [SMILES: O=C(NCCn1cccn1)c1cc(-c2ccc(Cl)s2)on1]

### N-(2-(1H-pyrazol-1-yl)ethyl)-5-(5-chlorothiophen-2-yl)isoxazole-3-carboxamide

General Procedure 5.1 (Scheme 5.1): step 1: 62% yield; step 2: 77% yield; step 3: 26% yield, as a yellow solid. Flash chromatography: (100% EtOAc).

¹H NMR (400 MHz, Chloroform-d) δ 7.58 (d, J=1.8 Hz, 2H), 7.40 (d, J=2.3 Hz, 2H), 7.38 (t, J=5.2 Hz, 2H), 7.31 (d, J=4.0 Hz, 2H), 6.97 (d, J=3.9 Hz, 2H), 6.75 (s, 2H), 6.27 (t, J=2.1 Hz, 2H), 4.34 (t, J=5.5 Hz, 3H), 3.91 (q, J=5.8 Hz, 3H). MS (ESI): 323.04 [M+H]⁺.

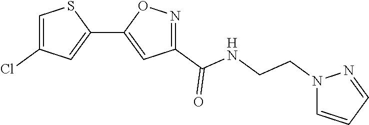 [SMILES: O=C(NCCn1cccn1)c1cc(-c2cc(Cl)cs2)on1]

### N-(2-(1H-pyrazol-1-yl)ethyl)-5-(4-chlorothiophen-2-yl)isoxazole-3-carboxamide

General Procedure 5.1 (Scheme 5.1): step 1: 67% yield; step 2: 93% yield; step 3: 85% yield, as a white solid. Flash chromatography: (100% EtOAc).

¹H NMR (400 MHz, Chloroform-d) δ 7.56 (d, J=1.6 Hz, 1H), 7.46 (t, J=6.1 Hz, 1H), 7.40 (d, J=2.1 Hz, 1H), 7.38 (d, J=1.5 Hz, 1H), 7.25 (d, J=1.5 Hz, 1H), 6.82 (s, 1H), 6.26 (t, J=2.1 Hz, 1H), 4.36 (t, J=5.5 Hz, 2H), 3.90 (q, J=5.8 Hz, 2H). MS (ESI): 323.04 [M+H]⁺.

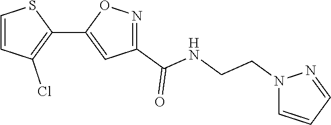 [SMILES: O=C(NCCn1cccn1)c1cc(-c2sccc2Cl)on1]

### N-(2-(1H-pyrazol-1-yl)ethyl)-5-(3-chlorothiophen-2-yl)isoxazole-3-carboxamide

General Procedure 5.1 (Scheme 5.1): step 1: 58% yield; step 2: 74% yield; step 3: 18% yield, as a white solid. Flash chromatography: (100% EtOAc).

¹H NMR (400 MHz, Chloroform-d) δ 7.59 (d, J=1.9 Hz, 1H), 7.47 (d, J=5.3 Hz, 1H), 7.41 (m, 2H), 7.27 (s, 1H), 7.08 (d, J=5.4 Hz, 1H), 6.28 (t, J=2.1 Hz, 1H), 4.39 (t, J=5.3 Hz, 2H), 3.94 (q, J=5.9 Hz, 2H). MS (ESI): 323.04 [M+H]⁺.

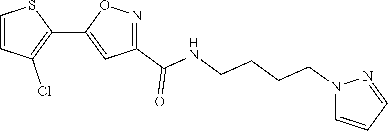 [SMILES: O=C(NCCCCn1cccn1)c1cc(-c2sccc2Cl)on1]

### N-(4-(1H-pyrazol-1-yl)butyl)-5-(3-chlorothiophen-2-yl)isoxazole-3-carboxamide

General Procedure 5.1 (Scheme 5.1): step 1: 70% yield; step 2: 83% yield; step 3: 30% yield, as a white solid. Flash chromatography: (Hexane/EtOAc=20/80).

¹H NMR (400 MHz, Chloroform-d) δ 7.52 (d, J=1.9 Hz, 1H), 7.46 (d, J=5.3 Hz, 1H), 7.39 (d, J=2.2 Hz, 1H), 7.25 (s, 1H), 7.06 (d, J=5.3 Hz, 1H), 7.00 (t, J=5.8 Hz, 1H), 6.25 (t, J=2.1 Hz, 1H), 4.20 (t, J=6.9 Hz, 2H), 3.46 (q, J=7.0 Hz, 2H), 1.97 (p, J=7.0 Hz, 2H), 1.62 (p, J=7.2 Hz, 2H). MS (ESI): 351.07 [M+H]⁺.

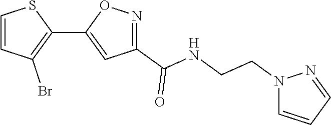 [SMILES: O=C(NCCn1cccn1)c1cc(-c2sccc2Br)on1]

### N-(2-(1H-pyrazol-1-yl)ethyl)-5-(3-bromothiophen-2-yl)isoxazole-3-carboxamide

General Procedure 5.1 (Scheme 5.1): step 1: 45% yield; step 2: 82% yield; step 3: 73% yield, as a white solid. Flash chromatography: (Hexane/EtOAc=30/70).

¹H NMR (400 MHz, Chloroform-d) δ 7.57 (d, J=1.6 Hz, 1H), 7.45 (m, 2H), 7.41 (d, J=2.3 Hz, 1H), 7.36 (s, 1H), 7.12 (d, J=5.3 Hz, 1H), 6.26 (t, J=2.1 Hz, 1H), 4.37 (t, J=5.6 Hz, 2H), 3.92 (q, J=5.8 Hz, 2H).

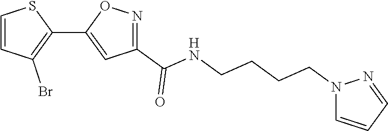 [SMILES: O=C(NCCCCn1cccn1)c1cc(-c2sccc2Br)on1]

### N-(4-(1H-pyrazol-1-yl)butyl)-5-(3-bromothiophen-2-yl)isoxazole-3-carboxamide

General Procedure 5.1 (Scheme 5.1): step 1: 45% yield; step 2: 82% yield; step 3: 38% yield, as a white solid. Flash chromatography: (100% EtOAc).

¹H NMR (400 MHz, Chloroform-d) δ 7.52 (d, J=1.7 Hz, 1H), 7.46 (d, J=5.3 Hz, 1H), 7.40 (d, J=2.3 Hz, 1H), 7.37 (s, 1H), 7.14 (d, J=5.3 Hz, 1H), 6.94 (t, J=5.5 Hz, 1H), 6.25 (t, J=2.1 Hz, 1H), 4.20 (t, J=6.9 Hz, 2H), 3.47 (q, J=6.8 Hz, 2H), 1.98 (p, J=6.9 Hz, 2H), 1.63 (p, J=7.2 Hz, 3H).

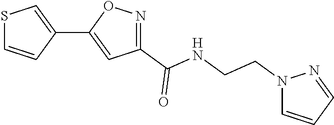 [SMILES: O=C(NCCn1cccn1)c1cc(-c2ccsc2)on1]

### N-(2-(1H-pyrazol-1-yl)ethyl)-5-(thiophen-3-yl)isoxazole-3-carboxamide

General Procedure 5.1 (Scheme 5.1): step 1: 53% yield; step 2: 74% yield; step 3: 62% yield, as a white solid. Flash chromatography: (100% EtOAc).

¹H NMR (400 MHz, Chloroform-d) δ 7.81 (dt, J=2.7, 1.1 Hz, 1H), 7.57 (d, J=1.6 Hz, 1H), 7.42 (m, 4H), 6.80 (s, 1H), 6.26 (t, J=1.7 Hz, 1H), 4.37 (t, J=5.2 Hz, 2H), 3.91 (q, J=6.0 Hz, 2H). MS (ESI): 289.07 [M+H]⁺.

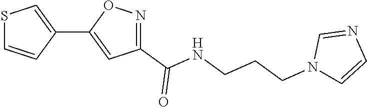 [SMILES: O=C(NCCCn1ccnc1)c1cc(-c2ccsc2)on1]

### N-(3-(1H-imidazol-1-yl)propyl)-5-(thiophen-3-yl)isoxazole-3-carboxamide

General Procedure 5.1 (Scheme 5.1): step 1: 58% yield; step 2: 83% yield; step 3: 54% yield, as a yellow solid. Flash chromatography: (EtOAc/MeOH=90/10).

¹H NMR (600 MHz, Chloroform-d) δ 7.83 (d, J=1.7 Hz, 1H), 7.59 (s, 1H), 7.43 (m, 2H), 7.08 (m, 2H), 7.00 (s, 1H), 6.81 (s, 1H), 4.06 (t, J=7.0 Hz, 2H), 3.47 (q, J=6.6 Hz, 2H), 2.13 (p, J=6.9 Hz, 2H). MS (ESI): 303.09 [M+H]⁺.

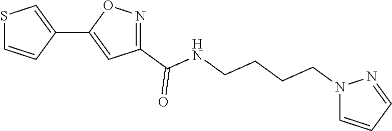 [SMILES: O=C(NCCCCn1cccn1)c1cc(-c2ccsc2)on1]

### N-(4-(1H-pyrazol-1-yl)butyl)-5-(thiophen-3-yl)isoxazole-3-carboxamide

General Procedure 5.1 (Scheme 5.1): step 1: 53% yield; step 2: 74% yield; step 3: 43% yield, as a white solid. Flash chromatography: (100% EtOAc).

¹H NMR (400 MHz, Chloroform-d) δ 7.81 (dd, J=2.9, 1.3 Hz, 1H), 7.51 (d, J=1.9 Hz, 1H), 7.41 (m, 3H), 6.99 (t, J=6.3 Hz, 1H), 6.80 (s, 1H), 6.24 (t, J=2.1 Hz, 1H), 4.19 (t, J=6.9 Hz, 2H), 3.46 (q, J=6.8 Hz, 2H), 1.97 (p, J=7.0 Hz, 2H), 1.62 (p, J=7.3 Hz, 1H). MS (ESI): 317.11 [M+H]⁺.

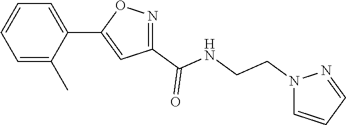 [SMILES: Cc1ccccc1-c1cc(C(=O)NCCn2cccn2)no1]

### N-(2-(1H-pyrazol-1-yl)ethyl)-5-(o-tolyl)isoxazole-3-carboxamide

General Procedure 5.1 (Scheme 5.1): step 1: 76% yield; step 2: 82% yield; step 3: 75% yield, as a white solid. Flash chromatography: (100% EtOAc).

¹H NMR (400 MHz, Chloroform-d) δ 7.72 (d, J=8.0 Hz, 1H), 7.58 (d, J=1.9 Hz, 1H), 7.45 (t, J=6.4 Hz, 1H), 7.42 (d, J=2.3 Hz, 1H), 7.37 (d, J=7.1 Hz, 1H), 7.31 (m, 2H), 6.87 (s, 1H), 6.27 (t, J=2.1 Hz, 1H), 4.38 (t, J=5.5 Hz, 1H), 3.93 (q, J=5.9 Hz, 2H), 2.51 (s, 3H). MS (ESI): 297.13 [M+H]⁺.

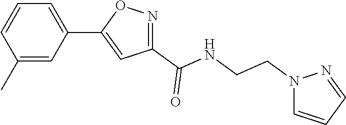 [SMILES: Cc1cccc(-c2cc(C(=O)NCCn3cccn3)no2)c1]

### N-(2-(1H-pyrazol-1-yl)ethyl)-5-(m-tolyl)isoxazole-3-carboxamide

General Procedure 5.1 (Scheme 5.1): step 1: 50% yield; step 2: 62% yield; step 3: 74% yield, as a pale-yellow solid. Flash chromatography: (100% EtOAc).

¹H NMR (400 MHz, Chloroform-d) δ 7.42 (m, 3H), 7.26 (s, 1H), 7.24 (d, J=2.3 Hz, 1H), 7.19 (t, J=7.6 Hz, 1H), 7.11 (d, J=8.8 Hz, 1H), 6.76 (s, 1H), 6.10 (t, J=2.0 Hz, 1H), 4.21 (t, J=5.6 Hz, 2H), 3.75 (q, J=5.9 Hz, 2H), 2.25 (s, 3H). MS (ESI): 297.13 [M+H]⁺.

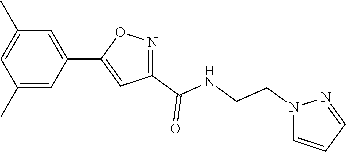 [SMILES: Cc1cc(C)cc(-c2cc(C(=O)NCCn3cccn3)no2)c1]

### N-(2-(1H-pyrazol-1-yl)ethyl)-5-(3,5-dimethylphenyl)isoxazole-3-carboxamide

General Procedure 5.1 (Scheme 5.1): step 1: 49% yield; step 2: 67% yield; step 3: 43% yield, as a white solid. Flash chromatography: (Hexane/EtOAc=30/70).

¹H NMR (400 MHz, Chloroform-d) δ 7.59 (d, J=1.9 Hz, 1H), 7.42 (d, J=2.3 Hz, 1H), 7.40 (s, 2H), 7.35 (t, J=5.9 Hz, 1H), 7.10 (s, 1H), 6.90 (s, 1H), 6.28 (t, J=2.1 Hz, 1H), 4.38 (m, 2H), 3.92 (q, J=5.8 Hz, 2H), 2.38 (s, 6H).

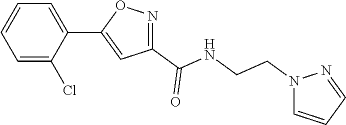 [SMILES: O=C(NCCn1cccn1)c1cc(-c2ccccc2Cl)on1]

### N-(2-(1H-pyrazol-1-yl)ethyl)-5-(2-chlorophenyl)isoxazole-3-carboxamide

General Procedure 5.1 (Scheme 5.1): step 1: 60% yield; step 2: 80% yield; step 3: 38% yield, as a pale-yellow solid. Flash chromatography: (100% EtOAc).

¹H NMR (400 MHz, Chloroform-d) δ 7.93 (dd, J=6.0, 3.5 Hz, 1H), 7.59 (d, J=1.9 Hz, 1H), 7.53 (dd, J=5.9, 3.5 Hz, 1H), 7.41 (m, 4H), 7.36 (s, 1H), 6.28 (t, J=2.2 Hz, 1H), 4.39 (t, J=5.4 Hz, 2H), 3.94 (q, J=5.9 Hz, 2H). MS (ESI): 317.08 [M+H]⁺.

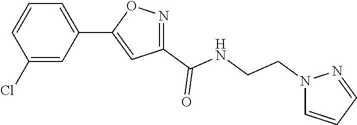 [SMILES: O=C(NCCn1cccn1)c1cc(-c2cccc(Cl)c2)on1]

### N-(2-(1H-pyrazol-1-yl)ethyl)-5-(3-chlorophenyl)isoxazole-3-carboxamide

General Procedure 5.1 (Scheme 5.1): step 1: 94% yield; step 2: 74% yield; step 3: 47% yield, as a white solid. Flash chromatography: (100% EtOAc).

¹H NMR (400 MHz, Chloroform-d) δ 7.77 (dt, J=1.7, 1.1 Hz, 1H), 7.66 (dt, J=6.7, 1.8 Hz, 1H), 7.58 (d, J=1.7 Hz, 1H), 7.43 (m, 4H), 6.98 (s, 1H), 6.27 (t, J=2.1 Hz, 1H), 4.38 (t, J=5.5 Hz, 2H), 3.92 (q, J=5.8 Hz, 2H). MS (ESI): 317.08 [M+H]⁺.

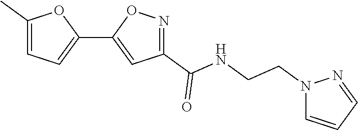 [SMILES: Cc1ccc(-c2cc(C(=O)NCCn3cccn3)no2)o1]

### N-(2-(1H-pyrazol-1-yl)ethyl)-5-(5-methylfuran-2-yl)isoxazole-3-carboxamide

General Procedure 5.1 (Scheme 5.1): step 1: 59% yield; step 2: 82% yield; step 3: 46% yield, as a white solid. Flash chromatography: (100% EtOAc).

¹H NMR (400 MHz, Chloroform-d) δ 7.57 (d, J=1.9 Hz, 1H), 7.40 (d, J=2.3 Hz, 1H), 7.35 (t, J=5.6 Hz, 1H), 6.82 (d, J=3.4 Hz, 1H), 6.76 (s, 1H), 6.26 (t, J=2.1 Hz, 1H), 6.13 (dd, J=3.4, 1.0 Hz, 1H), 4.37 (t, J=5.7 Hz, 2H), 3.90 (q, J=5.9 Hz, 2H), 2.38 (d, J=1.0 Hz, 3H). MS (ESI): 287.11 [M+H]⁺.

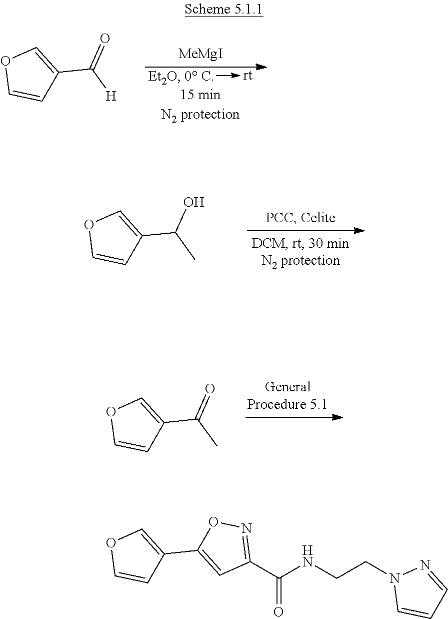

### 1-(Furan-3-yl)ethan-1-ol

To a solution of furan-3-carbaldehyde (360 μL, 4.16 mmol) in ether (10 mL), methyl magnesium iodide (3.0 M in ether) (2.08 mL, 6.24 mmol) was added dropwise at 0° C. under N₂ balloon protection. The reaction was stirred at room temperature for 15 minutes and was monitored by TLC. When complete, the mixture was quenched with saturated NH₄Cl solution. The aqueous layer was extracted with ether three times. The combined organic layers were dried over NaSO₄, filtered, and evaporated. The residue (364 mg, 78% yield) was used without further purification.

¹H NMR (400 MHz, Chloroform-d) δ 7.37 (m, 2H), 6.41 (t, J=1.4 Hz, 1H), 4.85 (q, J=6.4 Hz, 1H), 1.47 (d, J=6.5 Hz, 3H).

### 1-(Furan-3-yl)ethan-1-one

A mixture of pyridinium chlorochromate (PCC) (770 mg, 3.57 mmol) and celite (1:1 w/w, 770 mg) was added in several portions to a solution of 1-(furan-3-yl)ethan-1-ol (364 mg, 3.25 mmol) in DCM (8 mL). The reaction was stirred at room temperature and was monitored by TLC. When no starting material was observed (approximately 30 minutes), the mixture was diluted with ether (8 mL) and stirred for 15 minutes. The resulting suspension was filtered and the filtrate was evaporated. The crude material was purified via flash column chromatography (Hexane/EtOAc=95/5) to provide the title compound (102 mg, 23% yield of two steps).

¹H NMR (400 MHz, Chloroform-d) δ 8.02 (dd, J=1.3, 0.8 Hz, 1H), 7.44 (dd, J=1.9, 1.4 Hz, 1H), 6.77 (dd, J=1.9, 0.8 Hz, 1H), 2.44 (s, 3H).

### N-(2-(1H-pyrazol-1-yl)ethyl)-5-(furan-3-yl)isoxazole-3-carboxamide

1-(Furan-3-yl)ethan-1-one was used for isoxazole ring construction via General Procedure 5.1 (Scheme 5.1): step 1: 47% yield; step 2: 85% yield; step 3: 53% yield, as a white solid. Flash chromatography: (100% EtOAc).

¹H NMR (400 MHz, Chloroform-d) δ 7.94 (s, 1H), 7.57 (d, J=1.9 Hz, 1H), 7.52 (t, J=1.7 Hz, 1H), 7.40 (d, J=2.3 Hz, 1H), 7.38 (t, J=5.2 Hz, 1H), 6.73 (s, 1H), 6.70 (dd, J=1.9, 0.9 Hz, 1H), 6.26 (t, J=2.0 Hz, 1H), 4.37 (t, J=5.2 Hz, 2H), 3.91 (q, J=5.9 Hz, 2H). MS (ESI): 273.10 [M+H]⁺.

### General Procedure 5.2: Formation of Additional Ring Structures

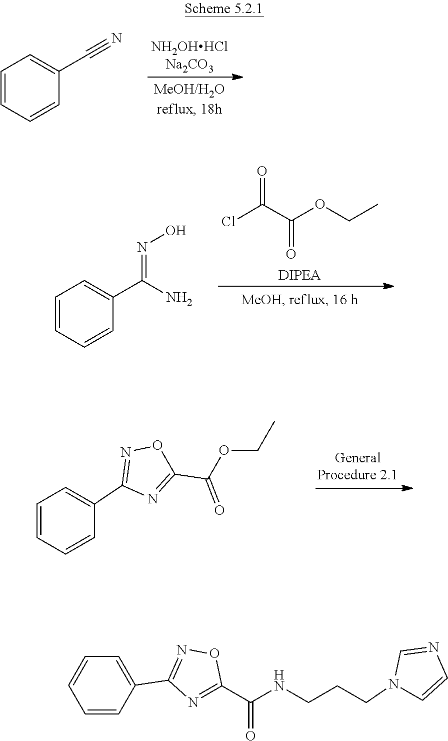

### N-Hydroxybenzimidamide

To a solution of benzonitrile (200 mg, 1.94 mmol) in methanol (4 mL) and water (0.8 mL), hydroxylamine hydrochloride (148 mg, 2.14 mmol) and sodium carbonate (103 mg, 0.97 mmol) were added. The mixture was refluxed for 18 hours. After cooling to room temperature, the solvent was evaporated and the residue was dissolved in EtOAc. The organics were washed with brine, dried over NaSO₄, and evaporated. The crude material as colorless oil (185 mg, 70% yield) was used for next step without further purification.

¹H NMR (600 MHz, Chloroform-d) δ 7.63 (m, 2H), 7.41 (m, 3H), 4.90 (s, 2H).

### Ethyl 3-phenyl-1,2,4-oxadiazole-5-carboxylate

To a solution of N-hydroxybenzimidamide (50 mg, 0.37 mmol) in methanol (1 mL), ethyl chloroglyoxylate (54 μL, 0.48 mmol) and DIPEA (68 μL, 0.48 mmol) were added. The mixture was refluxed for 16 hours. After cooling to room temperature, the solvent was evaporated. The crude material was purified via flash column chromatography (Hexane/EtOAc=80/20) to provide the ethyl ester (23 mg, 29% yield).

¹H NMR (400 MHz, Chloroform-d) δ 8.13 (m, 2H), 7.50 (m, 3H), 4.55 (q, J=7.1 Hz, 2H), 1.47 (t, J=7.1 Hz, 3H).

### N-(3-(1H-imidazol-1-yl)propyl)-3-phenyl-1,2,4-oxadiazole-5-carboxamide

Ethyl 3-phenyl-1,2,4-oxadiazole-5-carboxylate was used for amide bond formation reaction via General Procedure 2.1 (Scheme 2.1) to provide the title compound as a white solid (54% yield of two steps). Flash chromatography: (EtOAc/MeOH=90/10).

¹H NMR (400 MHz, Chloroform-d) δ 8.09 (m, 2H), 7.72 (s, 1H), 7.52 (m, 3H), 7.13 (s, 1H), 7.02 (s, 1H), 4.12 (t, J=6.8 Hz, 2H), 3.54 (q, J=6.6 Hz, 2H), 2.21 (p, J=6.8 Hz, 2H).

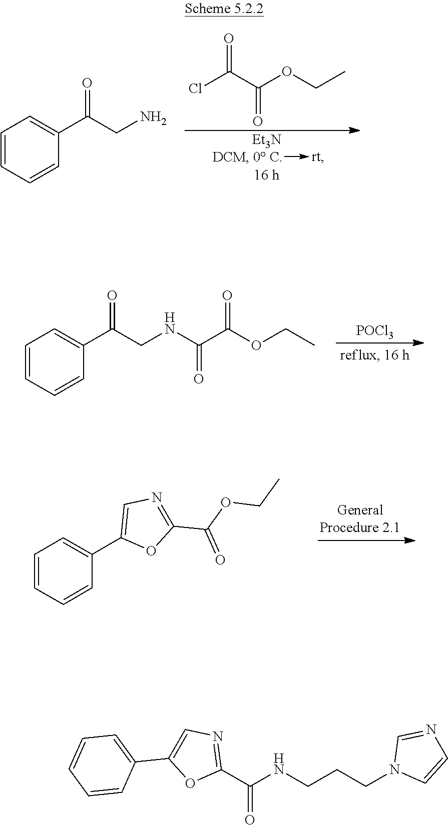

### Ethyl 2-oxo-2-((2-oxo-2-phenylethyl)amino)acetate

To a solution of 2-amino-1-phenylethan-1-one (200 mg, 1.17 mmol) in DCM (3 mL), ethyl chloroglyoxylate (143 μL, 1.28 mmol) and triethylamine (406 μL, 2.91 mmol) were added at 0° C. slowly. After stirring at room temperature for 16 hours, the mixture was acidified with 10% HCl in water to pH 3-4. The organics were washed with brine, dried over NaSO₄, and evaporated. The crude material was purified via flash column chromatography (EtOAc/MeOH=50/50) to provide the title compound (120 mg, 44% yield).

¹H NMR (400 MHz, Chloroform-d) δ 8.07 (s, 1H), 7.95 (m, 2H), 7.61 (tt, J=7.4, 1.3 Hz, 1H), 7.49 (t, J=8.0 Hz, 1H), 4.80 (d, J=4.7 Hz, 2H), 4.35 (q, J=7.1 Hz, 2H), 1.37 (t, J=7.2 Hz, 2H).

### Ethyl 5-phenyloxazole-2-carboxylate

The solution of ethyl 2-oxo-2-((2-oxo-2-phenylethyl)amino)acetate (50 mg, 0.21 mmol) in phosphorus oxychloride (1 mL) was refluxed for 16 hours. After cooling to room temperature, the solvent was evaporated and the residue was dissolved in DCM. The organics was washed with 5% NaHCO₃ and water, then dried over NaSO₄, filtered, and evaporated. The crude material was purified via flash column chromatography (EtOAc/MeOH=70/30) to provide the title compound (33 mg, 71% yield).

¹H NMR (400 MHz, Chloroform-d) δ 7.76 (m, 2H), 7.52 (s, 1H), 7.44 (m, 3H), 4.49 (q, J=7.2 Hz, 2H), 1.45 (t, J=7.2 Hz, 3H).

### N-(3-(1H-imidazol-1-yl)propyl)-5-phenyloxazole-2-carboxamide

Ethyl 3-phenyl-1,2,4-oxadiazole-5-carboxylate was used for amide bond formation reaction via General Procedure 2.1 (Scheme 2.1) to provide the title compound as colorless oil (30% yield of two steps). Flash chromatography: (EtOAc/MeOH=90/10).

¹H NMR (400 MHz, Chloroform-d) δ 7.75 (dd, J=7.5, 1.8 Hz, 2H), 7.69 (s, 1H), 7.45 (t, J=7.3 Hz, 2H), 7.40 (m, 2H), 7.31 (t, J=6.2 Hz, 1H), 7.11 (s, 1H), 7.02 (s, 1H), 4.09 (t, J=6.9 Hz, 2H), 3.49 (q, J=6.5 Hz, 2H), 2.15 (p, J=6.8 Hz, 2H).

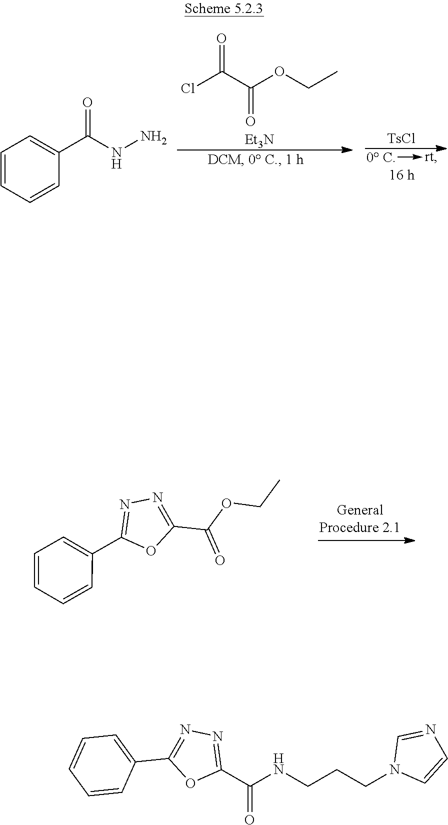

### Ethyl 5-phenyl-1,3,4-oxadiazole-2-carboxylate

To a solution of benzohydrazide (200 mg, 1.47 mmol) in DCM (4 mL), triethylamine (615 μL, 4.41 mmol) and ethyl chloroglyoxylate (164 μL, 1.47 mmol) were added slowly at 0° C. After stirring at 0° C. for 1 hour, p-toluenesulfonyl chloride (280 mg, 1.47 mmol) was added portion-wise. The reaction was warmed up to room temperature and stirred for 16 hours. The resulting mixture was washed with saturated NaHCO₃ and water, dried over NaSO₄, filtered, and evaporated. The crude material was purified via flash column chromatography (EtOAc/MeOH=80/20) to provide the title compound (108 mg, 34% yield) as a pale-yellow solid.

¹H NMR (600 MHz, Chloroform-d) δ 8.14 (m, 2H), 7.58 (m, 1H), 7.52 (m, 2H), 4.54 (q, J=7.2 Hz, 2H), 1.47 (t, J=7.2 Hz, 3H).

### N-(3-(1H-imidazol-1-yl)propyl)-5-phenyl-1,3,4-oxadiazole-2-carboxamide

Ethyl 5-phenyl-1,3,4-oxadiazole-2-carboxylate was used for amide bond formation reaction via General Procedure 2.1 (Scheme 2.1) to provide the title compound as a white solid (40% yield of two steps). Flash chromatography: (EtOAc/MeOH=90/10).

¹H NMR (400 MHz, Chloroform-d) δ 8.13 (d, J=7.1 Hz, 2H), 7.95 (t, J=6.2 Hz, 1H), 7.68 (s, 1H), 7.58 (m, 1H), 7.52 (m, 2H), 7.10 (s, 1H), 7.01 (s, 1H), 4.11 (t, J=6.9 Hz, 2H), 3.55 (q, J=6.6 Hz, 2H), 2.20 (p, J=6.9 Hz, 2H).

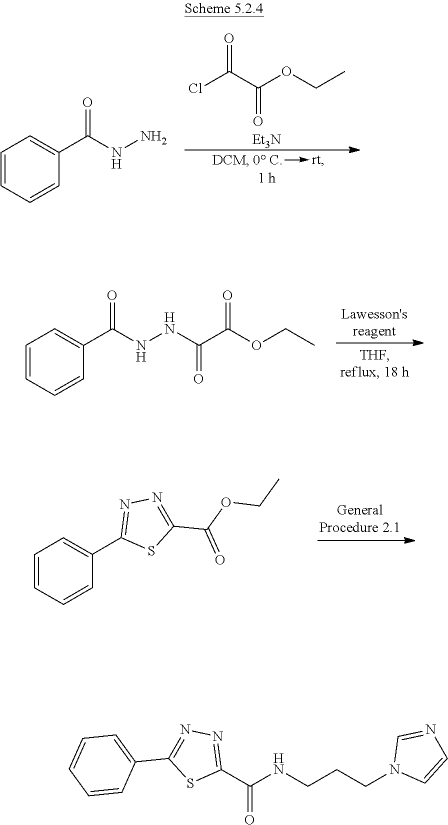

### Ethyl 2-(2-benzoylhydrazineyl)-2-oxoacetate

To a solution of benzohydrazide (200 mg, 1.47 mmol) in DCM (4 mL), triethylamine (615 μL, 4.41 mmol) and ethyl chloroglyoxylate (164 μL, 1.47 mmol) were added slowly at 0° C. The reaction was stirred at room temperature for 1 hour and monitored by TLC. The resulting mixture was washed with water, dried over NaSO₄, filtered, and evaporated. The crude material was purified via flash column chromatography (100% EtOAc) to provide the title compound (121 mg, 35% yield).

¹H NMR (400 MHz, Chloroform-d) δ 9.83 (d, J=6.7 Hz, 1H), 8.86 (s, 1H), 7.84 (dd, J=7.4, 1.9 Hz, 2H), 7.59 (t, J=7.3 Hz, 1H), 7.49 (t, J=7.6 Hz, 2H), 5.30 (m, 6H), 4.43 (q, J=7.5 Hz, 2H), 1.42 (t, J=7.1 Hz, 2H).

### Ethyl 5-phenyl-1,3,4-thiadiazole-2-carboxylate

To the solution of ethyl 2-(2-benzoylhydrazineyl)-2-oxoacetate (121 mg, 0.51 mmol) in THF (2 mL), Lawesson's reagent (269 mg, 0.66 mmol) was added. The mixture was refluxed for 18 hours. After cooling to room temperature, the solvent was evaporated. The residue was purified via flash column chromatography (EtOAc/MeOH=80/20) to provide the title compound (92 mg, 77% yield).

¹H NMR (400 MHz, Chloroform-d) δ 7.99 (m, 2H), 7.51 (m, 1H), 7.47 (ddd, J=8.5, 6.5, 1.5 Hz, 2H), 4.51 (q, J=7.1 Hz, 2H), 1.45 (t, J=7.1 Hz, 3H).

### N-(3-(1H-imidazol-1-yl)propyl)-5-phenyl-1,3,4-thiadiazole-2-carboxamide

Ethyl 5-phenyl-1,3,4-thiadiazole-2-carboxylate was used for amide bond formation reaction via General Procedure 2.1 (Scheme 2.1) to provide the title compound as a white solid (37% yield of two steps). Flash chromatography: (EtOAc/MeOH=90/10).

¹H NMR (400 MHz, Chloroform-d) δ 7.99 (d, J=6.7 Hz, 2H), 7.86 (s, 1H), 7.65 (t, J=5.9 Hz, 1H), 7.53 (m, 3H), 7.26 (s, 1H), 7.15 (s, 1H), 7.05 (s, 1H), 4.14 (t, J=6.9 Hz, 2H), 3.55 (q, J=6.6 Hz, 2H), 2.20 (p, J=6.8 Hz, 2H).

Additional Synthesis with Unique Procedure

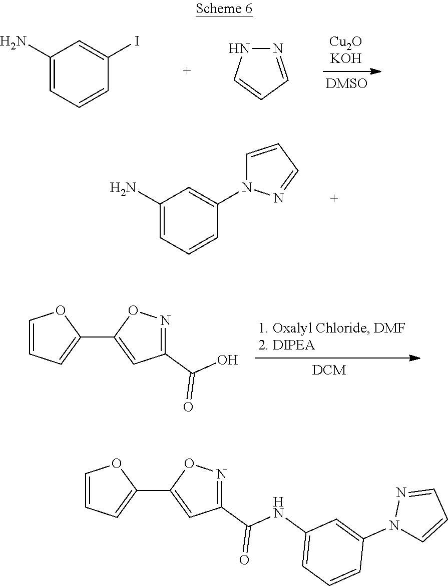 [SMILES: Nc1cccc(I)c1 ; c1cn[nH]c1 ; Nc1cccc(-n2cccn2)c1 ; O=C(O)c1cc(-c2ccco2)on1 ; O=C(Nc1cccc(-n2cccn2)c1)c1cc(-c2ccco2)on1]

### 3-(1H-pyrazol-1-yl)aniline

To a solution of pyrazole (102 mg, 1.5 mmol), 3-iodoaniline (219 mg, 1 mmol) and sodium hydroxide (112 mg, 2 mmol) in DMSO was added Copper (I) oxide (14.3 mg, 0.1 mmol) and stir at 125° C. for 16 hours. The resulting solution was filtered through celite and extracted with EtOAc three times. Combined organic was washed with saturated brine and dried with Na₂SO₄. Flash chromatography (Hexane/EtOAc: up to 50% EtOAc) to yield 36.3 mg product (22.8%) as black oil.

¹H NMR (400 MHz, Chloroform-d) δ 7.88 (dq, J=2.4, 0.8 Hz, 1H), 7.69 (dt, J=1.8, 0.9 Hz, 1H), 7.20 (tt, J=8.0, 0.9 Hz, 1H), 7.10 (td, J=2.2, 0.8 Hz, 1H), 7.00 (ddq, J=8.0, 1.8, 0.9 Hz, 1H), 6.59 (ddq, J=7.9, 2.6, 0.9 Hz, 1H), 6.43 (ddd, J=2.6, 1.7, 0.8 Hz, 1H).

### N-(3-(1H-pyrazol-1-yl)phenyl)-5-(furan-2-yl)isoxazole-3-carboxamide

To a solution of 5-(furan-2-yl)isoxazole-3-carboxylic acid (43 mg, 0.234 mmol) in DCM at 0° C. was added slowly 2M oxalyl chloride in DCM solution (117 μL, 0.234 mmol) and 10 μL DMF. The mixture was refluxed for 2 hours before being cooled to 0° C. To the cooled mixture was added 3-(1H-pyrazol-1-yl)aniline and diisopropylethylamine (61.3 μL, 0.352 mmol). The mixture was refluxed for 3 hours before purification with flash chromatography (Hexane/EtOAc: up to 75% EtOAc). 28.5% yield (21.4 mg) as an off-white solid.

¹H NMR (400 MHz, Chloroform-d) δ 8.66 (s, 1H), 8.14 (t, J=2.1 Hz, 1H), 7.98 (d, J=2.5 Hz, 1H), 7.74 (d, J=1.8 Hz, 1H), 7.63-7.51 (m, 3H), 7.46 (t, J=8.0 Hz, 1H), 7.00 (d, J=3.5 Hz, 1H), 6.95 (s, 1H), 6.58 (dd, J=3.5, 1.8 Hz, 1H), 6.49 (t, J=2.4 Hz, 1H).

Mice. C57BL/6J (JAX Stock #000664), B6.129(Cg)-Gt(ROSA)26Sortm4(ACTB-tdTomato,-EGFP)Luo/J (mTmG, JAX stock #007676), B6.129S-Cybbtm1Din/J (gp91phox-, JAX Stock #002365), NOD.Cg-Prkdcscid Il2rgtmlWjl (NSG, JAX stock #05557) and B6J.129(Cg)-Gt(ROSA)26Sortm1.1(CAG-cas9*,-EGFP)Fezh/J (CAG-Cas9-EGFP, JAX stock #026179) mice were purchased from Jackson Laboratory. All mice (male and female) were used between the age of 6 and 12 weeks old. All animal experiments were performed in accordance with protocols approved by the University of Southern California Animal Care and Use Committee. Animals (5 mice per cage) were provided food and water and were maintained on a regular 12-h light-dark cycle. NSG mice were bred under sterile condition

CGD mouse model. gp91phox-mice (CGD mice) were irradiated with a lethal dose (950 cGy) and transplanted with either 5×10⁶ tdTomato-positive GMPs and 2.5×10⁴ gp91phox-whole bone marrow cells (helper cells) or 2.5×10⁴ helper cells only via tail vein injection. Two days after transplantation, mice were injected intraperitoneally with 2×10⁸ S aureus strain 502A (ATCC No. 27217; ATCC) or 200 B cepacia bacilli (ATCC No. 25609; ATCC). The number of bacteria in the inoculum was confirmed by serial dilutions and plating. PBS or 5×10⁶ tdTomato-positive GMPs were injected via tail vein immediately after inoculation of bacteria and injection was repeated every 3 days thereafter. Mice were examined daily and euthanized if moribund or 7 days after peritoneal challenge. The presence of intraperitoneal abscesses was assessed by visual inspection. In some experiments, blood cultures were obtained from tail vein blood samples, and bacteremia was quantitated by plate culture.

Medium and reagents. DMEM/F-12 (12400024) and Neurobasal (21103049) media were purchased from Thermo Fisher Scientific. Human insulin (91077C-250MG), human Holo-transferrin (T0665-100MG), putrescine (P5780-5G), sodium selenite (S9133-1MG), linoleic acid (L1012-100MG), DL-alpha tocopherol (vit E, T3251-5G), and bovine serum albumin (A8806-5G) were purchased from Sigma. Recombinant murine SCF (250-03), recombinant human M-CSF (300-25), and recombinant human G-CSF (300-23) were purchased form PeproTech. GDC-0879 (S1104) and SKL2001 (S8302) were purchased from Selleck.

To prepare B7 medium, 500 ml of DMEM/F-12 and 500 mL of Neurobasal media were mixed and supplemented with 4 mg human insulin, 20 mg human Holo-Transferin, 16 mg putrescine, 12.5 μg sodium selenite, 1 mg linoleic acid, 1 mg vit E, and 2.5 g bovine serum albumin. Insulin does not dissolve readily; dissolve insulin in sterile 0.01 M HCl overnight at 4° C. to produce a 10 mg/mL stock solution. Store in 1-mL aliquots at −20° C. The suspension was mixed well before aliquoting.

Mouse GMP derivation, expansion, and differentiation. Cells were cultured at 37° C. in a 5% CO₂ water jacket incubator (Thermo Scientific). Bone marrow cells isolated from C57BL/6J, mTmG or CAG-Cas9-EGFP mice were plated into 6-well plates at a density of 2×10⁶ cells/well and cultured in 2 mL B7 medium supplemented with 50 ng/mL SCF, 1 μM GDC-0879, and 10 μM SKL2001 (SCF/2i). After 3-4 days, cells were dissociated into single-cell suspension by pipetting up and down and replated into 6-well plates at a density of 2×10⁶ cells/well and cultured in 2 mL B7 medium supplemented with SCF/2i. After 2 passages in SCF/2i, the majority of cells were GMPs. GMPs were routinely passaged every 3 days. To induce differentiation, GMPs were plated into 10 cm tissue culture dishes and cultured in RPMI-1640 medium containing 10% FBS and supplemented with either 20 ng/mL M-CSF (for macrophage differentiation) or 20 ng/mL G-CSF (for granulocyte differentiation). GMP-derived macrophages were harvested on day 7 (medium was changed once on day 4) and GMP-derived granulocytes were harvested on day 3 and used for the further experiments.

To generate bone marrow-derived macrophages, 2×10⁶ bone marrow cells isolated from the C57BL/6J mouse were plated into a 10 cm tissue culture dish and cultured in RPMI-1640 medium containing 10% FBS and 20 ng/ml M-CSF. The medium was changed on day 4 and cells were harvested on day 7.

Peritoneal macrophages were generated by injection of 1 mL of 2% Bio-Gel P-100 (Bio-Rad, 1504174) into the mouse peritoneal cavity immediately after transplantation of tdTomato-positive GMPs, followed by peritoneal lavage with sterile PBS 4 days later. Cells collected from the peritoneal cavity were used for fluorescence imaging and flow cytometry analysis.

Human GMP cell derivation and expansion. Human cord blood samples were obtained from StemCyte (Baldwin Park, CA), human whole bone marrow was purchased from Stemcell Technologies (Cat #70502.2) and human mobilized peripheral blood was purchased from StemExpress (Cat #MLE4GCSF5). Mononuclear cells were isolated using the Ficoll-Paque™ PLUS kit (GE Healthcare Life Sciences, 17-1440-03). Briefly, the blood was diluted with PBS at 1:3 ratio and added into SepMate™-50 tubes (Stemcell Technologies, 85460) preloaded with 15 ml Ficoll-Paque™ PLUS. After centrifugation at 1200 g for 20 minutes at room temperature, the top layer was collected and centrifugated at 300×g for 10 minutes at 4° C. The residual red blood cells were removed by using ACK lysing buffer. Cells were used immediately or cryopreserved in liquid nitrogen.

For the expansion of human GMPs, Lin⁻ (CD3, CD14, CD19 and CD56) CD34⁺CD38⁺ CD45RA⁺ GMPs were sorted from mononuclear cells isolated from cord blood, whole bone marrow or mobilized peripheral blood. Sorted human GMPs were plated into 96-well plates at a density of 4×10⁴ cells/well and cultured in B6 medium supplemented with human SCF (50 ng/mL. AF-300-07, PeproTech), GDC-0879 (1 μM) and TN-2-30 (5 μM) (modified SCF/2i). 5 days after the initial plating, human GMPs were routinely passaged every 3 days by re-plating them into 48-well plates at a density of 1×10⁵ cells/well and cultured in the modified SCF/2i. Replacement of GDC-0879 with SB590885 (0.5 μM. S2220, Selleck) could slightly increase human GMP proliferation rate. To prepare B6 medium, 500 mL of DMEM/F-12 and 500 ml of Neurobasal media are mixed and supplemented with 4 mg human insulin, 20 mg human Holo-Transferrin, 12.5 μg sodium selenite, 1 mg linoleic acid, 1 mg vit E, and 2.5 g human serum albumin.

Human leukemia cell derivation. Clinical specimens were obtained from adult B-cell acute lymphoblastic leukemia (B-ALL) patients. Human B-ALL cells were isolated from B-ALL patients' bone marrow aspirates by sorting for human CD45⁺ and CD19⁺ cells. Human B-ALL cells were transduced with GFP lentivirus. Cells were transplanted into NSG mice, and GFP leukemia cells were sorted from mouse spleens 6 weeks after transplantation.

GMP transplantation. GMPs were derived from mTmG mice and cultured in SCF/2i. After 3 passages, Ex vivo expanded GMPs were transplanted into C57BL/6J mice via tail vein injection at 1×10⁷ cells/mouse. Cells from blood, spleen and bone marrow were collected at the designated timepoints and stained with DAPI, CD11b-FITC, Ly6G-PerCP-Cy5.5 and CD115-PE-Cy7 antibodies and analyzed by flow cytometry. Liver tissues were harvested, fixed and sectioned for immunostaining.

Flow cytometry analysis and cell sorting. SCF/2i GMPs were collected and stained with cKit, FcgR, Sca1, CD34, B220, Ter119, CD3, and CD11b antibodies and analyzed by FACS-ArialI (BD Biosciences).

Mouse bone marrow cells were obtained from the crushed bones of mice using PBS with 2% (v/v) FBS. Bone debris were removed by density gradient centrifugation using Histopaque 1119 (Sigma). The cells were then enriched with CD117/cKit microbeads (Miltenyi Biotec) and IL7Ra antibody followed by anti-Rat IgG microbeads (Miltenyi Biotec). After staining with monoclonal antibodies, stem and progenitor populations were sorted using FACS-ArialI instrument. Cell-surface markers for each stem/progenitor cell lineage are summarized as follow:
HSC (hematopoietic stem cell): lineage (CD3, CD4, CD8, B220, Gr1, Mac1, Ter119)⁻/cKit⁺/Sca1⁺/Flk2⁺/CD34⁻/CD150⁺.
CLP (common lymphoid progenitor): lin⁻/IL7Rα⁺/Flk2⁺.
CMP (common myeloid progenitor): lin⁻/IL7Rα⁻/ckit⁺/Sca1⁻/CD34⁺/FcgR⁻.
MEP (megakaryotic/erythroid progenitor): lin⁻/IL7Rα⁻/ckit⁺/Sca1⁻/CD34⁻/FcgR⁻.
GMP (granulocyte/macrophage progenitor): lin⁻/IL7Rα⁻/ckit⁺/Sca1⁻/CD34⁺/FcgR⁺.

Blood samples were collected from 6-8 weeks C57BL/6J mice. Red blood cells were removed by ACK (Ammonium-Chloride-Potassium) Lysing Buffer (ThermoFisher, A1049201). White blood cells were stained and sorted. Cell-surface markers for each cell type are summarized as follow:
Monocyte/macrophage: CD3⁻/B220⁻/CD11b⁺/CD115+.
Neutrophil: CD3⁻/B220⁻/CD11b⁺/Ly6G⁺/CD115⁻.
T cell: CD3⁺/TCRab⁺/B220⁻/CD11b⁻.
B cell: B220⁺/CD19⁺/CD3⁻/CD11b⁻.

Antibodies were obtained from eBioscience (part of ThermoFisher) and BioLegend (see TABLE 1 for a full list of antibodies). Flow cytometry data were analyzed using FlowJo software version 10.4.2 (Tree Start) and Diva software 8.0.1 (BD Biosciences).

TABLE 1
Resources

| | | |
|---|---|---|
| REAGENT or RESOURCE | SOURCE | IDENTIFIER |

Antibodies

| | | |
|---|---|---|
| human CD45-APC-eFluor 780 | ThermoFisher | Cat #47-0459-42 |
| human CD19-PE | ThermoFisher | Cat #12-0198-42 |
| human CD56-FITC | ThermoFisher | Cat #15-0567-42 |
| human CD14-PE-Cyanine5 | ThermoFisher | Cat #15-0149-42 |
| human CD3-PE-Cyanine5 | ThermoFisher | Cat #15-0038-42 |
| human CD34-FITC | ThermoFisher | Cat #11-0349-42 |
| human CD38-PE | ThermoFisher | Cat #12-0389-42 |
| human CD68 | ThermoFisher | Cat #14-0687-82 |
| human CD45RA-PE/Cyanine7 | BioLegend | Cat #304126 |
| human CD15-APC | BioLegend | Cat #301908 |
| human CD66b-PerCP/Cyanine5.5 | BioLegend | Cat #305108 |
| mouse Ly-6A/E (Sca1)-PE-Cy7 | BioLegend | Cat #108113 |
| mouse CD150 (SLAM)-PE | BioLegend | Cat #115903 |
| mouse CD45-Alexa 700 | BioLegend | Cat #110724 |
| mouse IL7Ra-BV510 | BioLegend | Cat #135033 |
| mouse CD3e-PerCP-Cyanine5.5 | ThermoFisher | Cat #35-0031-82 |
| mouse CD4-PerCP-Cyanine5.5 | ThermoFisher | Cat #45-0042-80 |
| mouse CD8-PerCP-Cyanine5.5 | ThermoFisher | Cat #45-0081-80 |
| mouse B220-PerCP-Cyanine5.5 | ThermoFisher | Cat #45-0452-80 |
| mouse CD11b-PerCP-Cyanine5.5 | ThermoFisher | Cat #45-0112-80 |
| mouse TER-119-PerCP-Cyanine5.5 | ThermoFisher | Cat #45-5921-80 |
| mouse Ly-6G (Gr1)-PerCP-Cyanine5.5 | ThermoFisher | Cat #45-5931-80 |
| mouse CD117 (c-kit)-APC-eFluor780 | ThermoFisher | Cat #47-1171-80 |
| mouse CD16/CD32 (FcRγ)-eFluor450 | ThermoFisher | Cat #48-0161-82 |
| mouse CD34-eFluor660 | ThermoFisher | Cat #50-0341-80 |
| mouse CD11b-Alexa Fluor 488 | ThermoFisher | Cat #53-0112-82 |
| mouse CD115 (c-fms)-PE-Cyanine7 | ThermoFisher | Cat #25-1152-80 |
| mouse F4/80 Antigen-eFluor570 | ThermoFisher | Cat #41-4801-80 |

Bacterial and Virus Strains

| | | |
|---|---|---|
| S aureus strain 502A | ATCC | ATCC 27217 |
| B cepacia bacilli | ATCC | ATCC 25609 |
| Biological Samples | | |
| Human cord blood | StemCyte | https://www.stemcyte.com |
| human whole bone marrow | Stemcell | Cat #70502.2 |
| | Technologies | |
| human mobilized peripheral blood | StemExpress | Cat # MLE4GCSF5 |

Chemicals, Peptides, and Recombinant Proteins

| | | |
|---|---|---|
| Protein Kinase Inhibitor Library I | MilliporeSigma | Cat #539744 |
| Protein Kinase Inhibitor Library | MilliporeSigma | Cat #539745 |
| II | | |
| Protein Kinase Inhibitor Library | MilliporeSigma | Cat #539746 |
| III | | |
| Protein Kinase Inhibitor Library | MilliporeSigma | Cat #539747 |
| IV | | |
| AG-825 | Cayman Chemical | Cat #10010243 |
| SCH 772984 | Cayman Chemical | Cat #942183-80-4 |
| GDC-0623 | Cayman Chemical | Cat #1168091-68-6 |
| LY3009120 | Cayman Chemical | Cat #1454682-72-4 |
| UCN-01 | Cayman Chemical | Cat #18130 |
| MG-132 | Selleck | Cat #S2619 |
| | Chemicals | |
| Cyclopamine | Selleck | Cat #S1146 |
| | Chemicals | |
| Go6983 | Selleck | Cat #S2911 |
| | Chemicals | |
| Rapamycin | Selleck | Cat #S1039 |
| | Chemicals | |
| XMD8-92 | Selleck | Cat #S7525 |
| | Chemicals | |
| SCH772984 | Selleck | Cat #S7101 |
| | Chemicals | |
| DB07268 | Selleck | Cat #S6740 |
| | Chemicals | |
| TCS JNK 5a | Selleck | Cat #S7508 |
| | Chemicals | |
| Tomatidine | Selleck | Cat #S9430 |
| | Chemicals | |
| SP 600125 | Selleck | Cat #S1460 |
| | Chemicals | |
| SB 239063 | Selleck | Cat #S7741 |
| | Chemicals | |
| VX702 | Selleck | Cat #S6005 |
| | Chemicals | |
| SB431542 | Selleck | Cat #S1067 |
| | Chemicals | |
| LDN193189 | Selleck | Cat #S2618 |
| | Chemicals | |
| CHIR-99021 | Selleck | Cat #S1263 |
| | Chemicals | |
| SKL2001 | Selleck | Cat #S8320 |
| | Chemicals | |
| Dabrafenib | Selleck | Cat #S2807 |
| | Chemicals | |
| SB590885 | Selleck | Cat #S2220 |
| | Chemicals | |
| GDC-0879 | Selleck | Cat #S1104 |
| | Chemicals | |
| PLX8394 | Selleck | Cat #S7965 |
| | Chemicals | |
| PLX7904 | Selleck | Cat #S7964 |
| | Chemicals | |
| Vemurafenib | Selleck | Cat #S1267 |
| | Chemicals | |
| Sorafenib | Selleck | Cat #S7397 |
| | Chemicals | |
| TAK-632 | Selleck | Cat #S7291 |
| | Chemicals | |
| DMHI | Selleck | Cat #S7146 |
| | Chemicals | |
| DAPT | Selleck | Cat #S2215 |
| | Chemicals | |
| iCRT14 | Selleck | Cat #S8704 |
| | Chemicals | |
| IWR1 | Selleck | Cat #S7086 |
| | Chemicals | |
| XAV939 | Selleck | Cat #S1180 |
| | Chemicals | |
| FH535 | Selleck | Cat #S7484 |
| | Chemicals | |
| Calyculin A | Selleck | Cat #S9038 |
| | Chemicals | |
| Wnt-C59 | Selleck | Cat #S7037 |
| | Chemicals | |
| H89 | Selleck | Cat #S1582 |
| | Chemicals | |
| Quercetin | Selleck | Cat #S2391 |
| | Chemicals | |
| A-8301 | Selleck | Cat #S7692 |
| | Chemicals | |
| Rottlerin | Selleck | Cat #S7862 |
| | Chemicals | |
| PS48 | Selleck | Cat #S7586 |
| | Chemicals | |
| BIX01294 | Selleck | Cat #S8006 |
| | Chemicals | |
| Anisomycin | Selleck | Cat #S7409 |
| | Chemicals | |
| Wnt agonist 1 | Selleck | Cat #S8178 |
| | Chemicals | |
| PD173074 | Selleck | Cat #S1264 |
| | Chemicals | |
| PD0325901 | Selleck | Cat #S1036 |
| | Chemicals | |
| SB216763 | Selleck | Cat #S1075 |
| | Chemicals | |
| Recombinant Human BDNF | PeproTech | Cat #450-02 |
| Recombinant Human BMP-4 | PeproTech | Cat #120-05ET |
| Recombinant Human CNTF | PeproTech | Cat #450-13 |
| Recombinant Human EGF | PeproTech | Cat #AF-100-15 |
| Recombinant Human FGF-4 | PeproTech | Cat #100-31 |
| Recombinant Human FGF-basic | PeproTech | Cat #100-18B |
| Recombinant Human Flt3-Ligand | PeproTech | Cat #300-19 |
| Recombinant Human G-CSF | PeproTech | Cat #300-23 |
| Recombinant Human GM-CSF | PeproTech | Cat #300-03 |
| Recombinant Human IGF-I | PeproTech | Cat #100-11 |
| Recombinant Human IL-2 | PeproTech | Cat #200-02 |
| Recombinant Human IL-3 | PeproTech | Cat #200-03 |
| Recombinant Human IL-4 | PeproTech | Cat #200-04 |
| Recombinant Human IL-6 | PeproTech | Cat #200-06 |
| Recombinant Human LIF | PeproTech | Cat #300-05 |
| Recombinant Human M-CSF | PeproTech | Cat #300-25 |
| Recombinant Human Noggin | PeproTech | Cat #120-10C |
| Recombinant Human PDGF-AA | PeproTech | Cat #100-13A |
| Recombinant Human SCF | PeproTech | Cat #300-07 |
| Recombinant Human TGF-α | PeproTech | Cat #100-16A |
| Recombinant Human/Murine/Rat | PeproTech | Cat #120-14E |
| Activin A | | |
| Recombinant Human/Murine/Rat BMP-2 | PeproTech | Cat #120-02 |
| Recombinant Murine G-CSF | PeproTech | Cat #250-05 |
| Recombinant Murine GM-CSF | PeproTech | Cat #315-03 |
| Recombinant Murine HGF | PeproTech | Cat #315-23 |
| Recombinant Murine IL-4 | PeproTech | Cat #214-14 |
| Recombinant Murine M-CSF | PeproTech | Cat #315-02 |
| Recombinant Murine SCF | PeproTech | Cat #250-03 |
| Recombinant Murine Sonic Hedgehog | PeproTech | Cat #315-22 |
| (Shh) | | |
| Recombinant Murine TNF-a | PeproTech | Cat #315-01A |
| Recombinant Murine TPO | PeproTech | Cat #315-14 |
| Recombinant Murine Wnt-3a | PeproTech | Cat #315-20 |

N2B27 medium components

| | | |
|---|---|---|
| Catalase | MilliporeSigma | Cat #C40-100MG |
| Glutathione reduced | MilliporeSigma | Cat #G6013-5G |
| Human Insulin | MilliporeSigma | Cat #91077C-50MG |
| Superoxide Dismutase | MilliporeSigma | Cat #S5395-75KU |
| Human Holo-Transferin | MilliporeSigma | Cat #T0665-100MG |
| T3 | MilliporeSigma | Cat #T6397-100MG |
| L-carnitine | MilliporeSigma | Cat #C0283-1G |
| Ethanolamine | MilliporeSigma | Cat #E9508-100ML |
| D+-galactose | MilliporeSigma | Cat #G0625-100G |
| Putrescine | MilliporeSigma | Cat #P5780-5G |
| Sodium selenite | MilliporeSigma | Cat #S9133-1MG |
| Corticosterone | MilliporeSigma | Cat #C2505-500MG |
| Linoleic acid | MilliporeSigma | Cat #L1012-100MG |
| Linolenic acid | MilliporeSigma | Cat #L2376-500MG |
| Progesterone | MilliporeSigma | Cat #P8783-1G |
| Retinol acetate | MilliporeSigma | Cat #R7882-1G |
| DL-alpha tocopherol (vit E) | MilliporeSigma | Cat #T3251-5G |
| DL-alpha tocopherol acetate | MilliporeSigma | Cat #T3001-10G |
| Oleic acid | MilliporeSigma | Cat #01383-1G |
| Pipecolic acid | MilliporeSigma | Cat #P2519-100MG |
| Biotin | MilliporeSigma | Cat #B4639-100MG |
| Bovine Serum Albumin | MilliporeSigma | Cat #A8806-5G |

Critical Commercial Assays

| | | |
|---|---|---|
| Chromium Single Cell 3′ Library & | 10X Genomics | Cat #PN-120267 |
| Gel Bead Kit v2 | | |
| MethoCult ™ GF M3434 medium | Stemcell | Cat #03434 |
| | Technologies | |
| KaryoMAX ™ Giemsa Stain Solution | ThermoFisher | Cat #10092013 |
| Invitrogen ™ TNF alpha Mouse | ThermoFisher | Cat #88-7324-22 |
| Uncoated ELISA Kit | | |
| Invitrogen ™ IL-6 Mouse Uncoated | ThermoFisher | Cat #88-7064-22 |
| ELISA Kit | | |
| Invitrogen ™ IL-10 Mouse Uncoated | ThermoFisher | Cat #50-112-5188 |
| ELISA Kit | | |
| Invitrogen ™ TNF alpha Human | ThermoFisher | Cat #88-7346-76 |
| Uncoated ELISA Kit | | |
| Invitrogen ™ IL-6 Human Uncoated | ThermoFisher | Cat #88-7066-88 |
| ELISA Kit | | |
| Invitrogen ™ IL-10 Human Uncoated | ThermoFisher | Cat #88-7106-76 |
| ELISA Kit | | |
| Invitrogen ™ IFN gamma Human ELISA | ThermoFisher | Cat #KHC4022 |
| Kit | | |
| Neutrophil MPO activity assay kit | Cayman | Cat #600620 |
| Phagocytosis assay kit | Cayman | Cat #500290 |
| Raw and analyzed data | This disclosure | GEO: GSE150344 |

Experimental Models: Organisms/Strains

| | | |
|---|---|---|
| Mouse: C57BL/6J | The Jackson | JAX: 000664 |
| | laboratory | |
| Mouse: B6.129(Cg)- | The Jackson | JAX: 007676 |
| Gt(ROSA)26Sortm4(ACTB-tdTomato,- | laboratory | |
| EGFP)Luo/J | | |
| Mouse: B6.129S-Cybbtm1Din/J | The Jackson | JAX: 002365 |
| (gp91phox-) | laboratory | |
| Mouse: NOD.Cg-Prkdcscid | The Jackson | JAX: 05557 |
| Il2rgtm1Wjl | laboratory | |
| Mouse: B6J.129(Cg)- | The Jackson | JAX: 026179 |
| Gt(ROSA)26Sortm1.1(CAG-cas9*,- | laboratory | |
| EGFP)Fezh/J | | |

Recombinant DNA

| | |
|---|---|
| CarP-RFP | This disclosure |
| Carp^{Fc19}-RFP | This disclosure |
| CarP^{zFc19}-RFP | This disclosure |
| αHer2 CarP | This disclosure |

Software and Algorithms

| | | |
|---|---|---|
| FlowJo software version 10.4.2 | Tree Start | [https://www].flowjo.com/solutions/flowjo/downloads/previous- |
| | | versions |
| Diva software 8.0.1 | BD Biosciences | |

Single-cell RNA-seq library preparation. FACS-sorted single cell suspensions were washed with ice cold 0.04% (w/v) BSA in PBS, then loaded onto 3′ library chips as per the manufacturer's protocol for the Chromium Single Cell 3′ Library (10× Genomics, V2). The 10× Genomics libraries were first sequenced on an Illumina NextSeq 500 aiming at a coverage of 5,000 raw reads per cell to estimate the cell numbers, before being sequenced deeper on an Illumina HiSeq 2500 aiming at a coverage of 50,000 raw reads per cell (paired-end; read1: 26 cycles; i7 index: 8 cycles; read 2: 98 cycles).

Single-cell RNA-seq data analysis. Raw sequencing data were pre-processed using the Cell Ranger pipeline (10× Genomics, v 2.1.0). Briefly, the ‘cell ranger count’ function was used for UMI quantification. The reads were aligned to the m10 reference genome provided by Cell Ranger. The following metrics was used to exclude poor-quality cells: 1) number of detected genes lower than 200, 2) number of detected genes greater than 6000, or 3) percentage of reads aligned to mitochondrion greater than 10%. Genes that were detected in less than 10 cells in each cell type were also excluded.

The raw-count datasets from difference cell types were integrated and analyzed by Seurat3 R package. Briefly, the count numbers per gene in individual cells were normalized and scaled, and the whole dataset was analyzed using t-SNE dimension reduction method embedded in Seurat3. Differential gene expression analysis was conducted by comparing one cell type against the other cell types. The top 50 most highly expressed genes from each of the five primary cell types were chosen and used for analysis by heatmap. Some genes were highly expressed in two or more cell types).

Colony forming assay. SCF/2i GMPs or GMPs directly isolated from fresh bone marrow were deposited into 96-well plates containing 100 μl MethoCult™ GF M3434 medium (Stem Cell Technologies, 03434) at a density of one cell/well. Pictures were taken, and colonies were visually scored after 7 days of culture.

Immunofluorescent staining. SCF/2i GMP-derived macrophages were plated into 48-well plates. 24 hours later, cells were fixed with 4% (w/v) PFA for 15 minutes. Cells were stained with CD11b-FITC (ThermoFisher, #11-0112-82) and F4/80-eFlour 570 (ThermoFisher, #41-4801-82) antibodies. Liver sections (4 μm) were stained with anti-RFP (Abcam, #ab62341), anti-F4/80 (ThermoFisher, #14-4801-82) and related secondary antibodies. DAPI was used for nuclear counterstaining and pictures were taken using Keyence BZ-X710 fluorescence microscopy (Keyence).

Giemsa staining and Karyotyping. SCF/2i GMPs and SCF/2i GMP-derived granulocytes were spread onto slides. After fixation, the cells were stained with 10% KaryoMAX™ Giemsa Stain Solution (ThermoFisher, 10092013) for 5 minutes. Slides were washed under tap water, and pictures were taken using Keyence BZ-X710 fluorescence microscopy (400×). For karyotyping, GMPs were expanded in SCF/2i medium for 8 passages. Cells were treated with 100 ng/mL colcemid (Sigma, 10295892001) for 2 hours before being harvested for metaphase preparation by standard methods. The GTW banding method was used for chromosome analysis as described in Hsieh, C. L. (Basic cytogenetic techniques: culture, slidemaking and banding. In Cell Biology: A Laboratory Handbook, Second Edition, JE Cells, ed. (New York: Academic Press), 391-396).

ELISA. Bone marrow-derived macrophages, SCF/2i GMP-derived macrophages, blood granulocytes, and SCF/2i GMP-derived granulocytes were plated into 48-well plates at a density of 1×10⁵ cells/well and cultured in RPMI 1640 medium supplemented with 10% FBS. Macrophages were cultured overnight prior to the start of stimulation with TLR ligands: 500 ng/mL LPS-EK (InvivoGen, tlrl-eklps). Granulocytes were stimulated immediately after plating. Cell culture supernatants were harvested after 6 hours of stimulation and the cytokines TNFα, IL-6 and IL-10 were measured using the Ready-SET-Go! ELISA kits (ThermoFisher).

Myeloperoxidase (MPO) assay. SCF/2i GMPs, blood neutrophils and SCF/2i GMP-derived granulocytes were plated into 96-well plates at a density of 2×10⁴ cells/well and cultured in 100 μl RPMI 1640 medium supplemented with 10% FBS. Cells were stimulated with 100 nM PMA for 2 hours with or without MPO inhibitor 4-aminobenzhydrozide (ABH). The supernatants were harvested and MPO activities were measured using the neutrophil MPO activity assay kit (Cayman, 600620) according to the manufacturer's instructions.

Phagocytosis assay. Phagocytosis of latex beads was performed using phagocytosis assay kit (Cayman, 500290). Briefly, tdTomato-positive SCF/2i mouse GMP-derived macrophages were plated into 48-well plates at a density of 5×10⁴ cells/well and cultured in DMEM/10% FBS overnight. Latex bead-rabbit IgG-FITC complex was added to cell cultures (1:100 dilution) and unbonded beads were washed out after one hour. Cells were harvested for flow cytometry analysis or fixed for DAPI staining and microscopy visualization.

For phagocytosis of bacteria, DHSa E. coli cells were transformed with TOPO-GFP plasmid and plated onto LB agar plates with ampicillin selection. GFP-positive bacteria colonies were picked and diluted in PBS after 16 hours. Macrophages derived from tdTomato-positive SCF/2i mouse GMPs were plated into 24-well plates at a density of 1×10¹ cells/well and cultured in DMEM/10% FBS overnight, after which 1×10¹ GFP-positive bacteria were added to each well. Consecutive images were taken every 30 seconds using the Zeiss LSM-780 or Keyence BZ-X710 microscopy.

For antibody treatment groups, human B-ALL cells were pre-incubated with either 20 μg/ml mouse IgG2a (Bio-Rad, MCA929) or anti-CD47 (Bio-Rad, MCA911) antibodies for 30 minutes before adding to the macrophage culture. Consecutive images were taken every 2.5 minutes using the Keyence BZ-X710 microscopy. Videos were created with the Keyence microscopy analyzer. To analyze phagocytic abilities, macrophages were washed, trypsinized and analyzed by flow cytometry. GFP and RFP double positive cells were considered effective for phagocytosis, and phagocytosis percentage was calculated by dividing the number of double positive cells by the total number of RFP-positive cells.

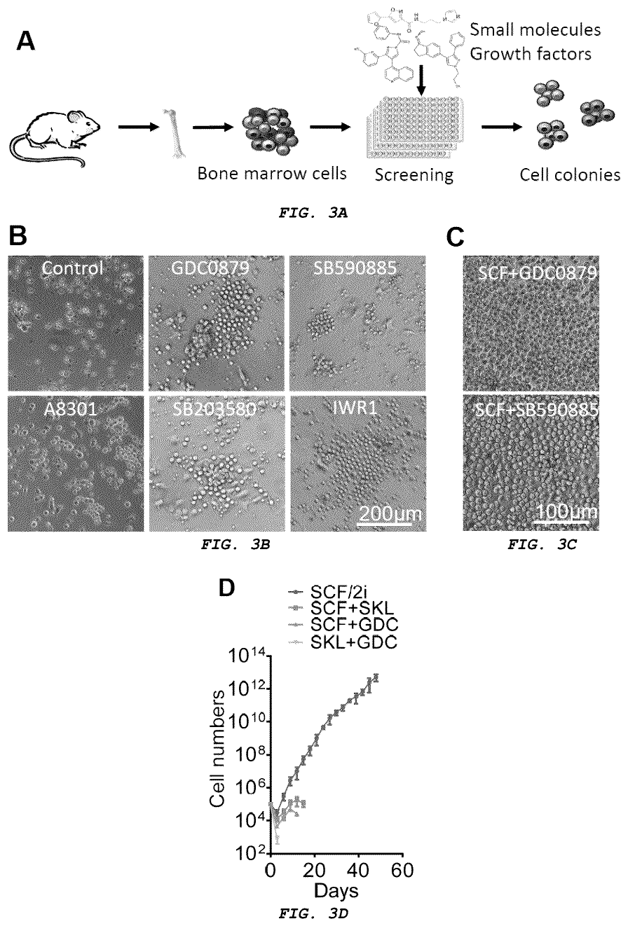

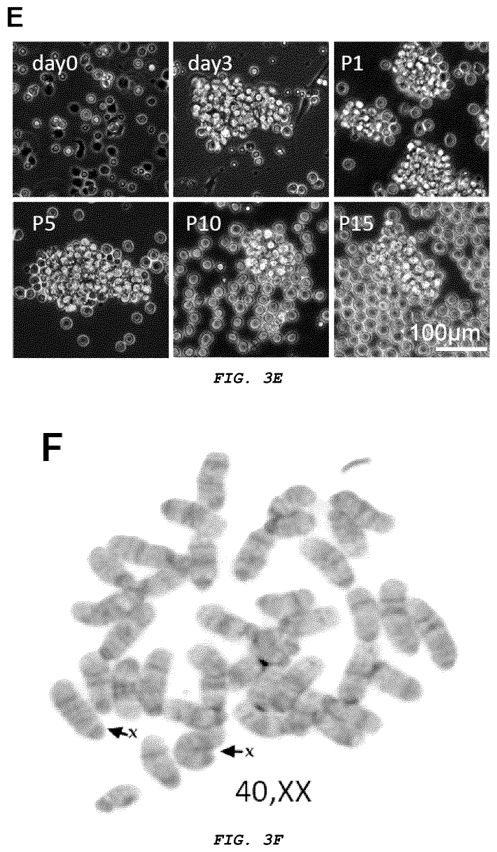

Development of defined conditions for the long-term ex vivo expansion of hematopoietic stem/progenitor cells. To develop conditions for the long-term expansion of stem or progenitor cells of the hematopoietic system, screening was conducted to identify small molecules and growth factors/cytokines that can promote their ex vivo expansion (see FIG. 3A). Specifically, bone marrow cells isolated from adult C57BL/6 mice were plated into 96-well plates in serum-free N2B27 medium. Then a first-round small molecule screening was performed. It was reasoned that if a small molecule can block differentiation or promote expansion of stem cells, these stem cells should form colonies with a uniform cell morphology. With 5 of the 375 small molecule inhibitors screened (see TABLE 1), clusters of colonies were observed being formed after 7 days in culture (GDC-0879, SB590885, A-8301, SB203580, and IWR1) (see FIG. 3B). Upon passaging, however, no new colonies were formed in culture with any of the 5 small molecule inhibitors. A second-round screening was conducted to identify growth factors/cytokines that could further improve the expansion of the colonies. Of the 32 growth factors/cytokines screened (see TABLE 1), it was found that stem cell factor (SCF), when combined with either GDC-0879 or SB590885, which are both potent B-Raf inhibitors, allowed the expansion of cells with a uniform cell morphology for 3-4 passages (see FIG. 3C), after which they gradually differentiated and stopped proliferating.

A third-round screening was then conducted with the remaining 373 small molecules in the presence of SCF and GDC-0879, a combination that proved slightly better than SCF and SB590885 for the expansion of the cells. SKL2001, a reported regulator of Wnt/(3-catenin pathway, was identified that allowed for the long-term expansion of a uniform bright and round-shaped cell population when combined with SCF and GDC-0879. N2B27 medium contains 22 key components (see TABLE 1). The N2B27 medium was further refined and it was found that a combination of just 7 of the 22 components was superior to N2B27. These 7 components were: bovine serum albumin, insulin, transferrin, putrescine, linoleic acid, sodium selenite, and vitamin E. In the presence of these 7 basal components, which is referred to as the B7 medium, the uniform cell population expanded exponentially while remaining karyotypically normal, when supplemented with SCF, GDC-0879, and SKL2001 (SCF/2i thereafter) (see FIGS. 3D-F). More importantly, these cells could be expanded robustly at clonal density (see FIG. 3G). SCF/2i is important for long-term expansion, as removal of any of the three factors resulted in cell death and differentiation (see FIG. 3D).

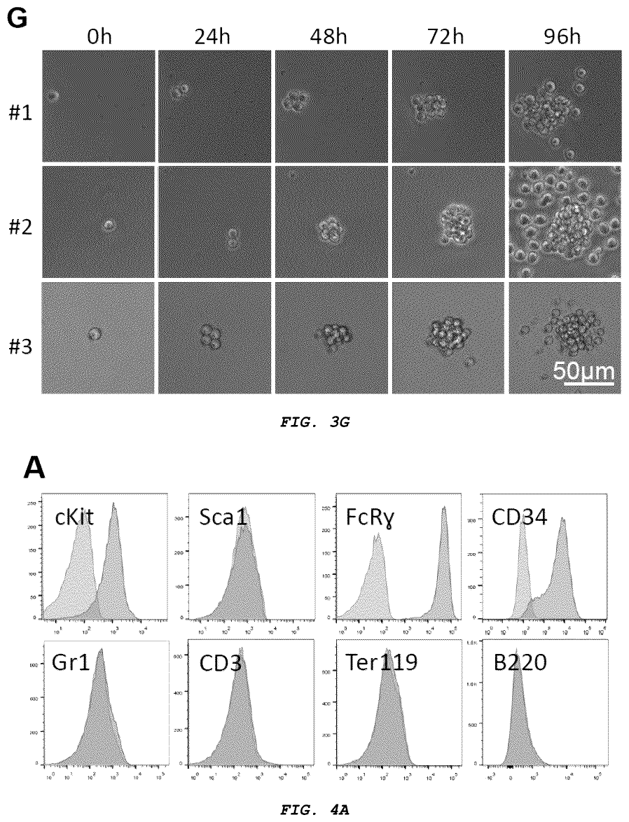

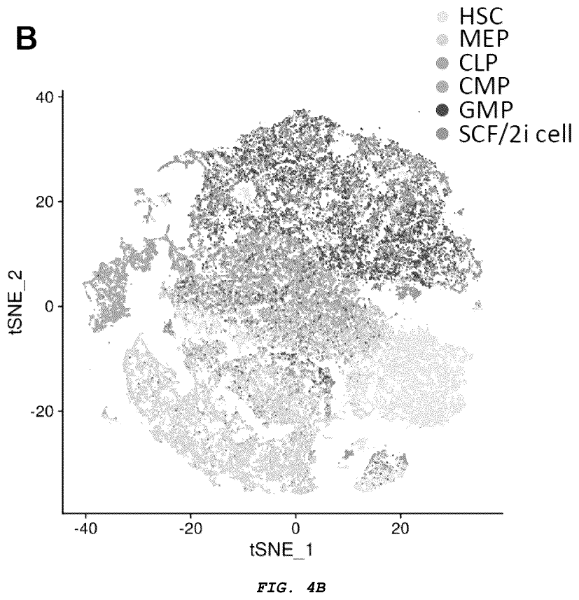

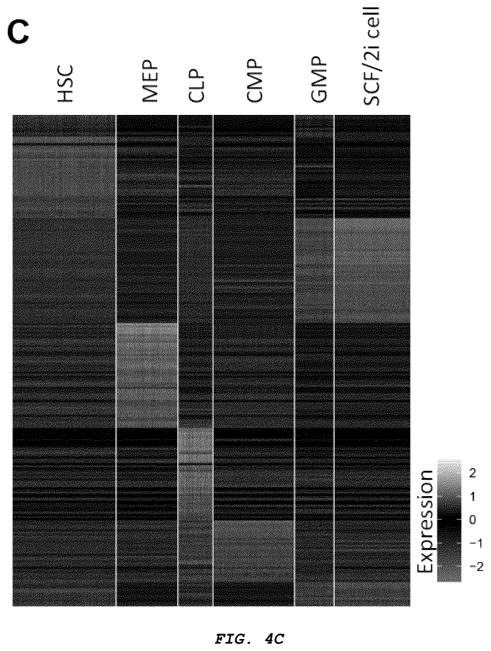

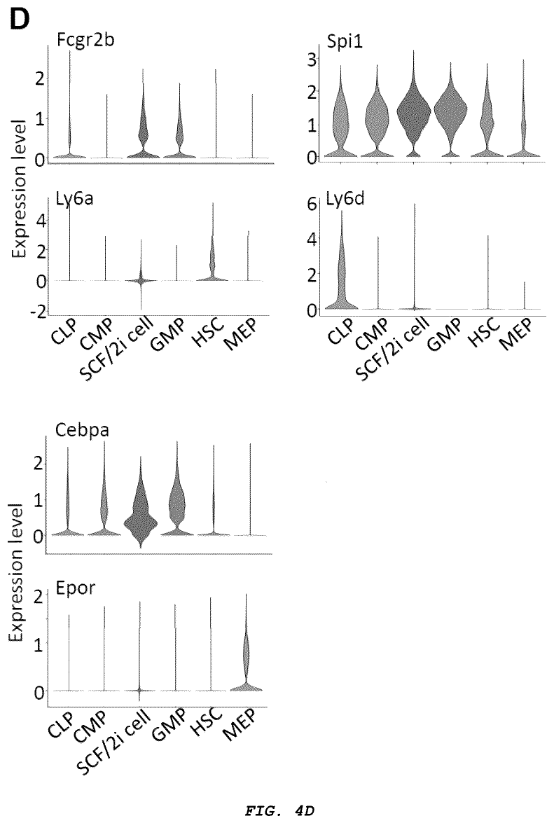

SCF/2i-expanded cells are GMPs. To determine the identity of the cells expanded in SCF/2i, different cell populations were isolated from mouse bone marrow by fluorescence-activated cell sorting (FACS). The isolated cells were then cultured in SCF/2i. Hematopoietic stem cells (HSCs), common myeloid progenitors (CMPs), and GMPs formed colonies with uniform bright and round-shaped cells that could be continuously expanded, but common lymphoid progenitors (CLPs), monocytes, granulocytes, T cells, and B cells did not. This suggests that the cells expanded in SCF/2i likely belong to the myeloid lineage. Further immunophenotypic analysis showed that SCF/2i cells were lin⁻ cKit⁺ Sca1⁻ FcRγ⁺ CD34⁺ (see FIG. 4A), indicating a GMP identity.

To further confirm the cells' identity, single-cell RNA sequencing (scRNA-seq) were performed on cells expanded in SCF/2i and freshly sorted hematopoietic stem and progenitor cells and found that the transcriptome of SCF/2i cells overlapped with that of freshly sorted GMPs (see FIG. 4B). SCF/2i cells shared a similar gene expression profile with GMPs, but not with other hematopoietic cell lineages (see FIGS. 4C-D). These results further support that SCF/2i cells are GMPs.

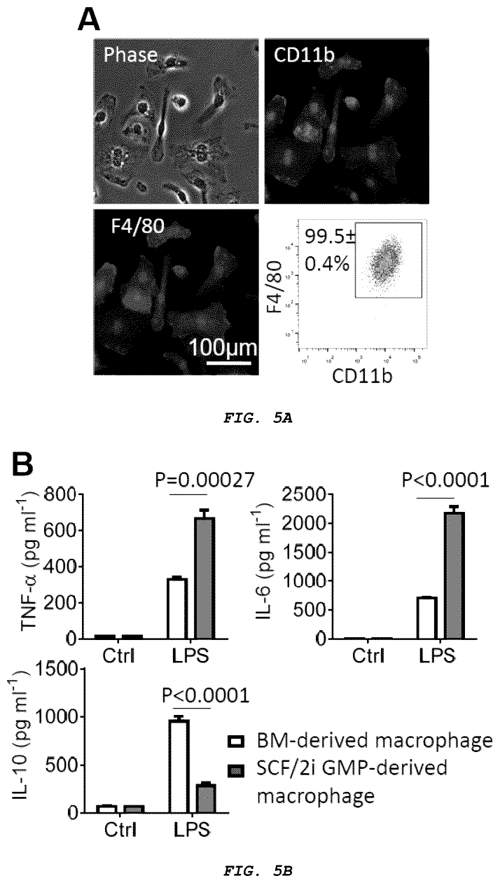

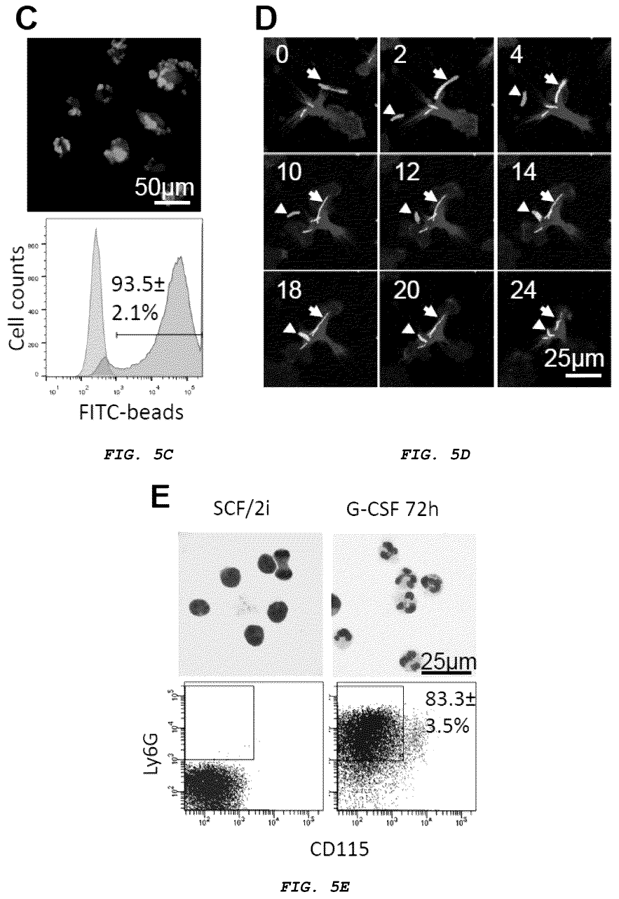

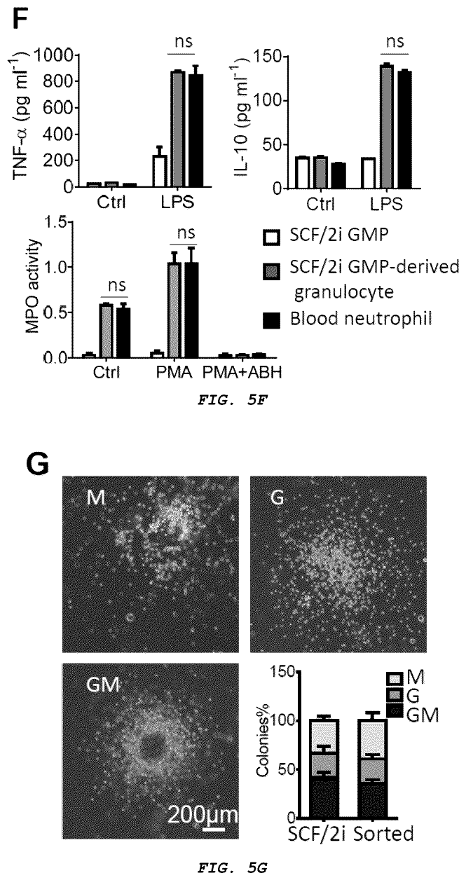

SCF/2i-expanded GMPs can efficiently differentiate into functional macrophages and granulocytes in vitro. In vitro differentiation assays were performed to functionally assess SCF/2i GMPs. Treatment of macrophage colony-stimulating factor (M-CSF), a macrophage differentiation factor, efficiently induced SCF/2i GMPs to differentiate into large and flat cells expressing F4/80 and CD11b, two key macrophage markers (see FIG. 5A). Flow cytometry analysis of the differentiated cells showed that more than 99% of cells were positive for both F4/80 and CD11b (see FIG. 5A). One of the key features of macrophages is the secretion of inflammatory cytokines when stimulated with lipopolysaccharide (LPS), a potent activator of macrophages. After LPS stimulation, macrophages derived from both SCF/2i GMPs and bone marrow produced abundant cytokines, including tumor necrosis factor (TNF)-α, interleukin (IL)-6, and IL-10 (see FIG. 5B). Compared to bone marrow-derived macrophages, the macrophages derived from SCF/2i GMPs produced significantly more of the pro-inflammatory cytokines TNF-α and IL-6, but less of the anti-inflammatory cytokine IL-10. This suggests that SCF/2i GMP-derived macrophages predominantly exhibit a pro-inflammatory (M1) phenotype.

Another key feature of macrophages is the ability to remove pathogens and cell debris through phagocytosis. A phagocytosis assay was conducted by incubating fluorescein isothiocyanate (FITC)-labeled latex beads with macrophages derived from SCF/2i GMPs. One hour after incubation, more than 90% of the cells had engulfed fluorescent beads (see FIG. 5C). Moreover, SCF/2i GMP-derived macrophages were also very efficient at engulfing and killing bacteria (see FIG. 5D).

To test their potential to differentiate into granulocytes, SCF/2i GMPs were treated with granulocyte colony stimulating factor (G-CSF) and phenotypic and functional assays were performed. After treatment with G-CSF for 3 days, SCF/2i GMPs differentiated into cells with a segmented nucleus, a characteristic of granulocytes (see FIG. 5E, upper panel). Flow cytometry analysis showed that more than 80% of these cells were Ly6G⁺CD115⁻, a characteristic phenotype of granulocytes (see FIG. 5E, lower panel). To assess whether SCF/2i GMP-derived granulocytes were functional their ability to secrete inflammatory cytokines in response to LPS stimulation was assessed. It was found that SCF/2i GMP-derived granulocytes produced abundant TNF-α and IL-10 in response to LPS stimulation, which was comparable to freshly isolated peripheral blood neutrophils (the major type of granulocyte) (see FIG. 5F). Another characteristic of granulocytes is the release of myeloperoxidase (MPO), a heme-containing peroxidase that, upon activation, mediates the killing of microbes. MPO activity was significantly increased in both SCF/2i GMP-derived granulocytes and blood neutrophils upon activation by phorbol myristate acetate (PMA), a protein kinase C (PKC) agonist (see FIG. 5F). Moreover, PMA-induced MPO activity in these cells was blocked by 4-aminobenzoic hydrazide (ABH), a potent MPO inhibitor (see FIG. 5F).

To more rigorously test differentiation potential of the SCF/2i GMPs, a colony-forming unit (CFU) assay was performed by depositing SCF/2i GMPs and freshly isolated GMPs into 96-well plates at a density of one cell per well. Among all the colonies formed from individual SCF/2i GMPs, 41.39±5.45%, 25.47±6.68%, and 33.14±4.46% were granulocyte/macrophage, granulocyte only, and macrophage only colonies, respectively. These results indicate a differentiation potential similar to that of GMPs isolated from bone marrow (see FIG. 3G).

Collectively, the above results demonstrate that ex vivo expanded SCF/2i GMPs can efficiently differentiate into phenotypic and functional granulocytes and macrophages.

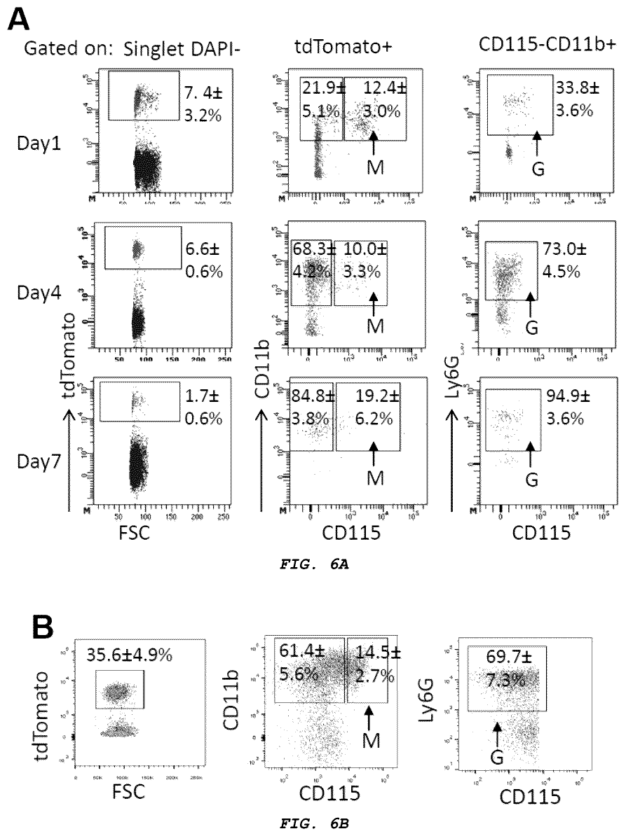

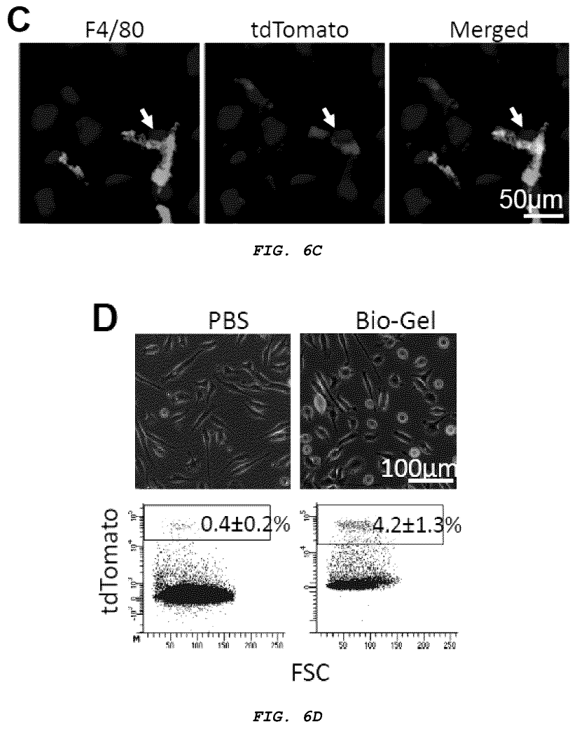

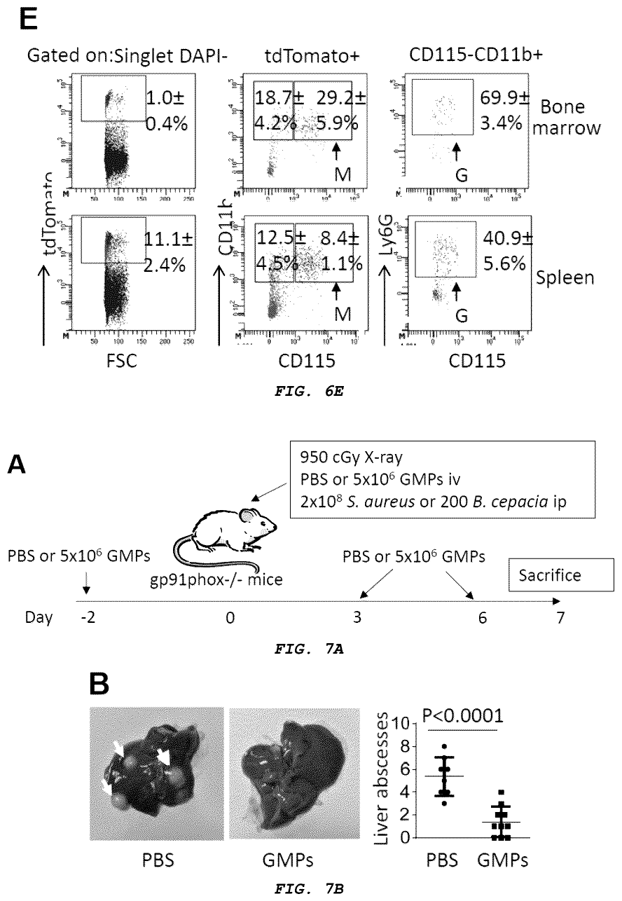

SCF/2i-expanded GMPs can efficiently differentiate into granulocytes and macrophages in vivo. To determine whether ex vivo expanded SCF/2i GMPs retain the ability to differentiate into granulocytes and macrophages in vivo, SCF/2i GMPs were derived from mice ubiquitously expressing the red fluorescent protein variant tdTomato and transplanted them into C57BL/6 mice via tail vein injection (1×10⁷ cells/mouse). Flow cytometry analysis of peripheral blood collected from mice 1, 4, and 7 days after transplantation showed that 7.4±3.2%, 6.6±0.6%, and 1.7±0.6% (n=6 mice) of white blood cells were tdTomato positive, respectively. Among the day 1 tdTomato positive cells, 12.4±3.0% and 7.5±2.6% were macrophages and granulocytes, respectively. On day 4, 10.0±3.3% were macrophages and 49.9±6.1% were granulocytes; on day 7, most of the tdTomato positive cells differentiated into either macrophages (19.2±6.2%) or granulocytes (80.5±6.7%) (see FIG. 6A).

Although readily detectable post-transplantation, the proportions of transplanted GMPs and their derivatives in the peripheral blood were relatively low as shown above. It was investigated whether the proportion of transplanted cells can be increased by administration of conditioning regimens such as total body irradiation before transplantation. Recipient mice were pretreated with sub-lethal irradiation before transplantation of tdTomato-positive GMPs (1×10⁷ cells/mouse) and peripheral blood collected from mice 4 days after transplantation was analyzed. The proportion of tdTomato positive cells in the peripheral blood dramatically increased to 35.6±4.9% of all white blood cells (see FIG. 6B). Among the tdTomato-positive cells, 14.5±2.7% and 43.1±8.3% were macrophages (CD11b⁺CD115⁺) and granulocytes (CD11b⁺CD115-Ly6G⁺), respectively. tdTomato-positive macrophages were also identified in the liver, peritoneal cavity, bone marrow, and spleen of recipient mice (see FIGS. 6C-4E), suggesting that transplanted GMPs can also differentiate into tissue macrophages.

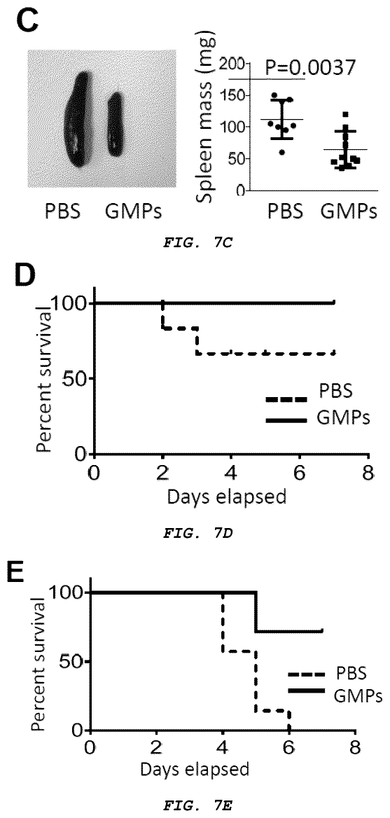

SCF/2i-expanded GMPs exhibited therapeutic effects in a mouse model of bacterial infection. The therapeutic effect of SCF/2i GMPs were evaluated using the chronic granulomatous disease (CGD) mouse model (see FIG. 7A). CGD mice are susceptible to infection due to a defect of granulocytes and macrophages in phagocyte microbicidal activity. Infusion of SCF/2i GMPs significantly decreased the number of liver abscesses (see FIG. 7B), reduced the size of spleens (see FIG. 7C), and lowered death rates (see FIG. 7D) of CGD mice inoculated with Staphylococcus aureus. Similar therapeutic effects of SCF/2i GMPs in CGD mice inoculated with another strain of bacteria, Burkholderia cepacian, were also observed (see FIGS. 7E-F).

Taken together, these results demonstrate that ex vivo expanded SCF/2i GMPs retain the ability to differentiate into functional granulocytes and macrophages after transplantation.

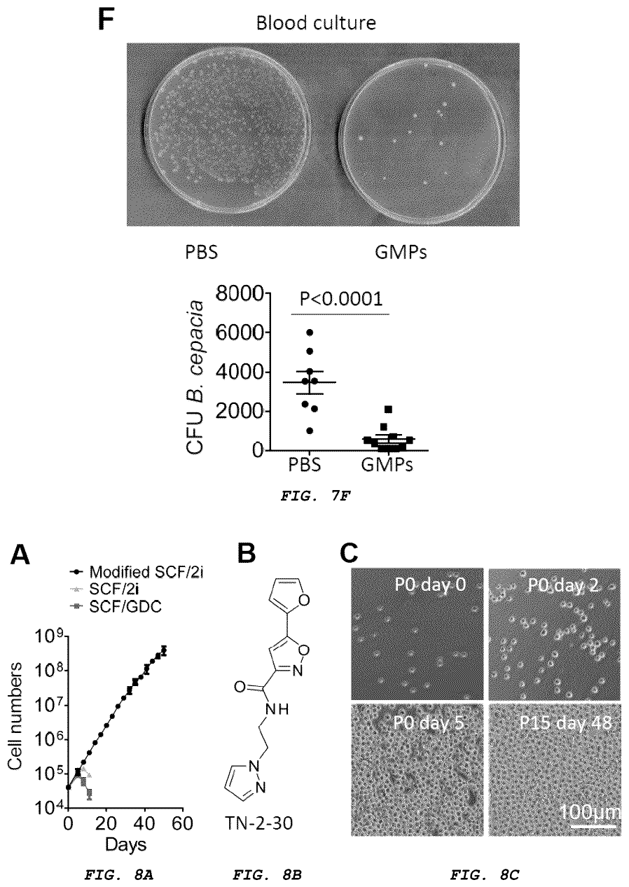

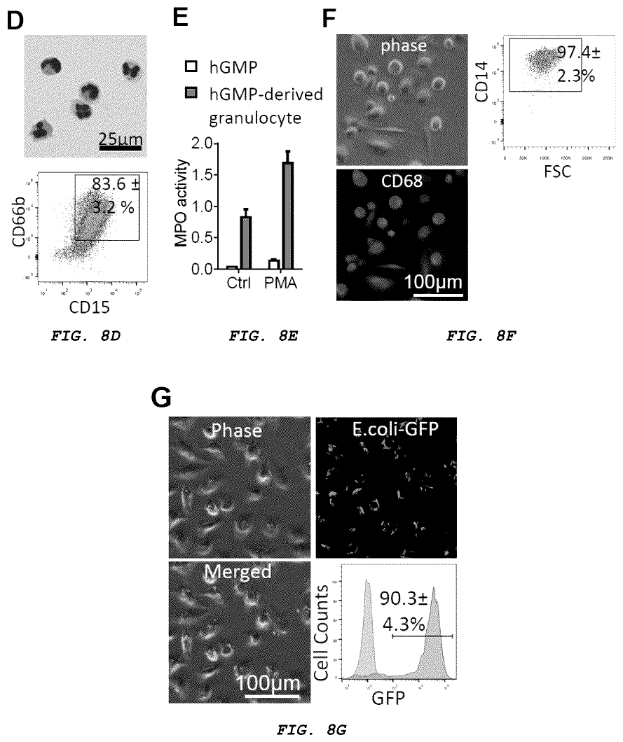

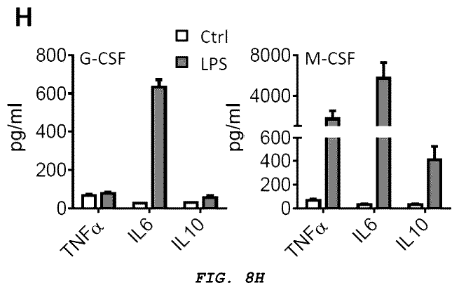

Human GMPs expand in a modified SCF/2i medium. To determine whether human GMPs could also expand ex vivo, lin⁻CD34⁺CD38⁺CD45RA⁺ GMPs were sorted from human cord blood and cultured them in the presence of SCF and small molecules. Sorted human GMPs could be passaged for 2-3 times in SCF/GDC-0879, after which they stopped proliferating and differentiated (see FIG. 8A), similar to mouse GMPs (see FIG. 3D), indicating that SCF/GDC-0879 retain their activities on human GMPs. Addition of SKL2001, however, only marginally improved the expansion of human GMPs (see FIG. 8A).

Next, was investigated why mouse and human GMPs respond differently to SKL2001. CHIR99021, Wnt agonist 1, and Wnt3a, three classic Wnt/β-catenin activators, could not mimic the effect of SKL2001 for the expansion of mouse GMPs. Expansion of mouse GMPs in SCF/2i was also not affected by Wnt/β-catenin signaling inhibitors IWR1 and FH535. Furthermore, expansion of β-catenin knockout mouse GMPs still required the addition of SKL2001. These results suggest that SKL2001 promotes expansion of GMPs through a Wnt/β-catenin-independent mechanism.

Encouraged by the result that human GMPs are also responsive to SKL2001 for expansion, albeit much more weakly compared to mouse GMPs, it was postulated that more effective analogues could be identified for the expansion of human GMPs by systematically modifying the structures of SKL2001. A total of 50 SKL2001 analogues were synthesized and characterized. One of the new SKL2001 analogues, TN-2-30 (see FIG. 8B), significantly improved expansion of human GMPs compared to SKL2001 (see FIG. 8A). Human GMPs expanded exponentially in the modified SCF/2i (TN-2-30 in place of SKL2001) (see FIGS. 8A-C) while retaining the ability to efficiently differentiate into phenotypic and functional granulocytes and macrophages (see FIGS. 8D-8H).

Characterization of human GMPs. To characterize the cultured GMPs, fresh bone marrow cells were first isolated and then a flow cytometer was used to sort out different hematopoietic stem/progenitor cells including hematopoietic stem cells (HSCs) (Lin-cKit⁺ Sca1⁺ Flk2⁻ CD34⁻ Slam⁺), GMPs (Lin⁻ cKit⁺ Sca1⁻ CD34⁺ FcgR⁺), monocytes (Mac1⁺ CD115⁺ B220⁻ TCRab⁻), granulocytes (Mac1⁺ CD115⁻ Gr1⁺ B220⁻ TCRab⁻), T cells (TCRab⁺ Gr⁻ Ma1c⁻ B220⁻) and B cells (B220⁺ CD19⁺ Gr1⁻ Mac1⁻ TCRab⁻). These different types of cells were cultured with a composition of the disclosure to determine which types of cells could be expanded and give rise to GMPs. HSCs and GMPs formed identical cell colonies and these cells were able to self-renew for long-term expansion when the compositions of the disclosure are used. After passage 3, the cells were harvested and the cell surface markers are checked by flow cytometer after staining. Cells that are cKit⁺ Sca1⁻ CD34⁺ FcgR⁺, indicate that they are GMPs. GMPs could give rise to granulocytes, macrophages, and dendritic cells. Next, an in vitro differentiation assay is performed to further characterize these ex vivo expanded GMPs.

Long-term ex vivo expanded GMPs can differentiate into functional and mature macrophages. To induce differentiation into mature macrophages, 1×10⁵ ex vivo expanded GMPs were plated per well into a 6-well plate and cultured in RPMI 1640+10% FBS+20 ng/mL MCSF. The cells proliferated, started to attach, and differentiated 3 days after plating. By day 7, the total cell number of cells increased to ˜2×10⁶. The cells were passaged and re-plated at 1×10⁵ cells per well in a 24-well plate. Twenty-four hours after plating, the cells are fixed and stained with the macrophage markers CD11b and F4/80, and DAPI for the nuclei. The cells expressed both CD11b and F4/80, suggesting that GMPs have been induced to differentiate into macrophages. As one major type of the innate immune cells, macrophages perform their functions by phagocytosis and secreting inflammatory cytokines. It is well-known that macrophages express high level of toll-like receptor 4 (TLR4) and the activation of TLR4 by LPS dramatically increases the production of inflammatory cytokines. To test whether macrophages derived from GMPs can secrete inflammatory cytokines, GMP-derived macrophages (GMPMs) or bone marrow-derived macrophages (BMMs) were plated at 1×10⁵ cells/well into 48-well plates and then stimulated with 500 ng/mL LPS. After 6 or 24h, the supernatant was harvested and the concentrations of inflammatory cytokines TNFα, IL6 and IL10 by ELISA were measured.

Long-term ex vivo expanded GMPs can differentiate into functional and mature granulocytes. Granulocyte colony-stimulating factor (G-CSF) is a hematopoietic growth factor that regulates neutrophils production in the bone marrow. G-CSF is used to induce mouse GMP differentiation towards the neutrophil lineage. After culturing for 3 weeks in E7⁺SCF/GDC/CHIR conditions, GMPs were re-plated in RPMI 1640+10% FBS medium and stimulated with 20 ng/mL of GCSF. Seventy-two hours after stimulation with GCSF, GMPs differentiated into cells that morphologically resembled granulocytes. To further verify the identity of these cells, the cells were harvested and stained with Gr1 and CD115 antibodies and analyzed by flow cytometer. Granulocytes are Gr1⁺ and CD115⁻.

Myeloperoxidase (MPO) is released from granulocytes/neutrophils to degrade invading pathogens, providing one of the earliest lines of defense in innate immunity. To functionally evaluate mouse GMP-derived granulocytes, MPO activity was measured using an MPO activity assay kit (Cayman Chemical Company). Mouse neutrophils (Gr1⁺CD11b⁺CD115⁻) were sorted from the whole blood using BD Arial I flow cytometer and used as a positive control. GMPs, GMP-derived granulocytes and blood-originated neutrophils were plated into 96-well plates at 1×10¹ cells/well in RPMI 1640 medium containing 1% BSA. Cells were then stimulated with 100 nM phorbol myristate acetate (PMA) for 2h and the MPO activities in the supernatants are measured following the manufacture's protocol. 4-aminobenzhydrazide (ABH), a specific inhibitor for MPO, was used to determine the specificity of the assay. MPO activity in undifferentiated GMPs could not be detected, while GMPs-derived granulocytes and blood neutrophils possessed similar MPO activities with PMA stimulation or without PMA stimulation.

Since neutrophils also express high levels of TLR4, the GMPs-derived granulocytes were stimulated with LPS to further evaluate their cytokine generating abilities. GMPs, GMPs-derived granulocytes and blood neutrophils were plated at 1×10⁵ cells/well into 96-well plates in RPMI 1640 medium containing 10% FBS. The cells were stimulated with 500 ng/mL LPS. Twenty-four hours later, the supernatants were collected and the inflammatory cytokines TNFα, IL6 and IL10 were measured by ELISA.

Genetic modification of ex vivo expanded GMPs. It is difficult to perform genetic modification in mature macrophages and granulocytes. Presented herein are methods developed for the efficient genetic modification in GMPs. A very highly efficient protocol to either overexpress or knockout a gene in GMPs is described. More than 95% of GMPs transfected with GFP mRNA were GFP positive. GFP and toll like receptor 4 (TLR4) genes were knocked out by a CRISPR/Cas9 system. Guide RNAs were specifically designed and synthesized so as to target GFP or toll like receptor 4 (TLR4) genes. gRNAs targeting GFP were introduced into GMPs. About 91.1% of the GMPs transfected with GFP gRNAs became GFP negative 48h after transfection. A similar efficiency was achieved in knocking out the TLR4 gene in GMPs. A GFP-GMP knockout and a TLR4-GMP knockout were differentiated into mature macrophages and stimulated with Poly I:C and LPS. Twenty-four hours later, the supernatant was harvested the amounts of inflammatory cytokines secreted by the cells were measured by ELISA.

It will be understood that various modifications may be made without departing from the spirit and scope of this disclosure. Accordingly, other embodiments are within the scope of the following claims.

1. A method for the expansion of a population of human granulocyte/macrophage progenitor cells (GMPs), comprising:
culturing GMPs in a culture medium comprising:
(i) a growth factor;
(ii) a B-Raf kinase inhibitor;
(iii) an agent that inhibits the mitogen-activated kinase interacting protein kinases 1 and 2 (Mnk1/2);
(iv) an agent that inhibits the PI3K pathway;
(v) a compound having the structure of Formula I: 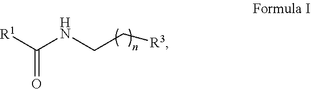
wherein,
R¹ is selected from: 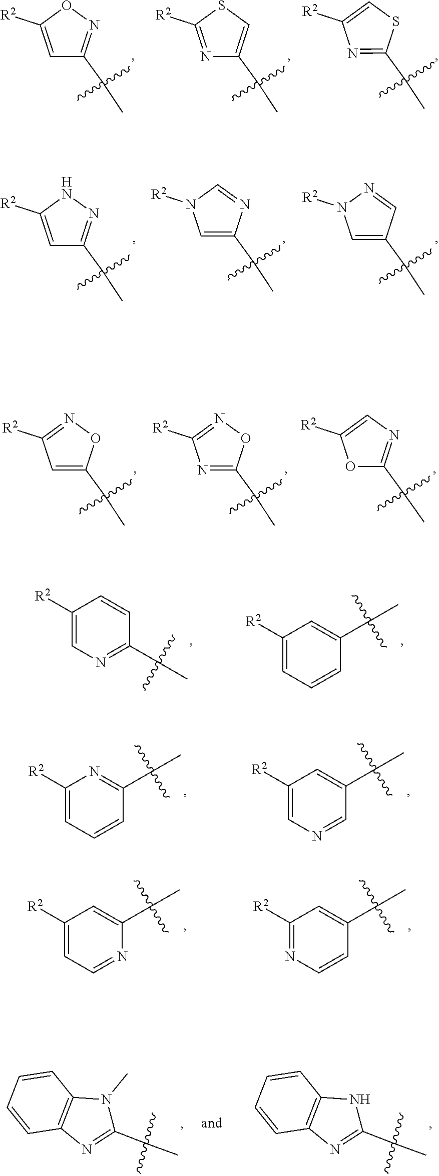
R² is selected from: 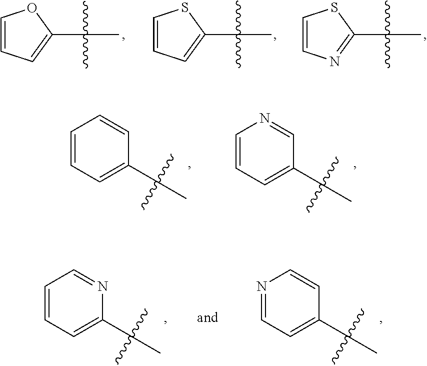
R³ is selected from: 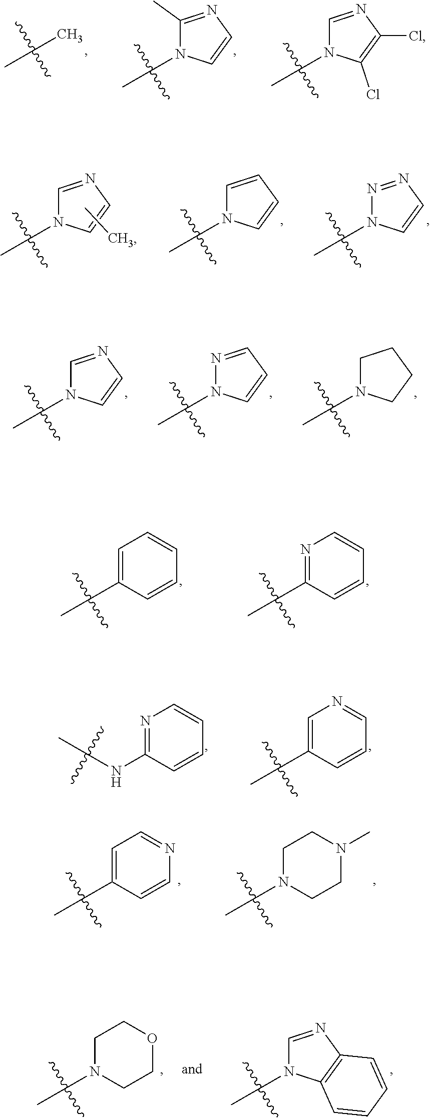
n is an integer selected from 0, 1, 2, 3, 4, and 5;
wherein the GMPs maintain their morphological characteristics after undergoing multiple cell passages and/or clonal expansion.

2. The method of claim 1, wherein the GMPs are derived or obtained from human stem cells.

3. The method of claim 2, wherein the human stem cells are genetically engineered prior to or during culturing.

4. The method of claim 2, wherein the human stem cells are hematopoietic stem cells.

5. The method of claim 4, wherein the hematopoietic stem cells are isolated from the bone marrow of a human subject.

6. The method of claim 1, wherein the culture medium comprises DMEM/F12 and Neural Basal Medium.

7. The method of claim 6, wherein the culture medium comprises DMEM/F12 and Neural Basal Medium in a ratio of about 5:1 to about 1:5.

8. (canceled)

9. The method of claim 1, wherein the culture medium comprises one or more supplements selected from insulin, transferrin, bovine serum albumin (BSA) fraction V, putrescine, sodium selenite, DL-α tocopherol, linolenic acid and/or linoleic acid.

10. (canceled)

11. The method of claim 1, wherein the growth factor is stem cell factor (SCF).

12. The method of claim 1, wherein the B-Raf kinase inhibitor is selected from the group consisting of GDC-0879, PLX4032, GSK2118436, BMS-908662, LGX818, PLX3603, RAF265, R05185426, vemurafenib, PLX8394, SB590885 and any combination thereof.

13. The method of claim 1, wherein the compound having the structure of Formula I is selected from:

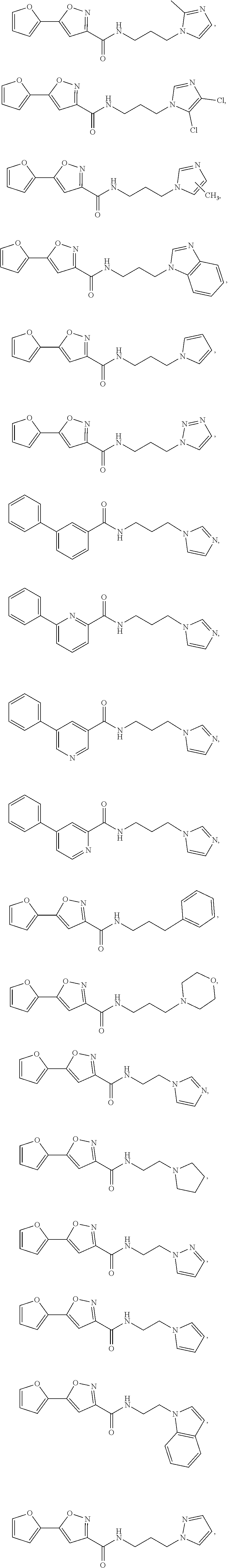

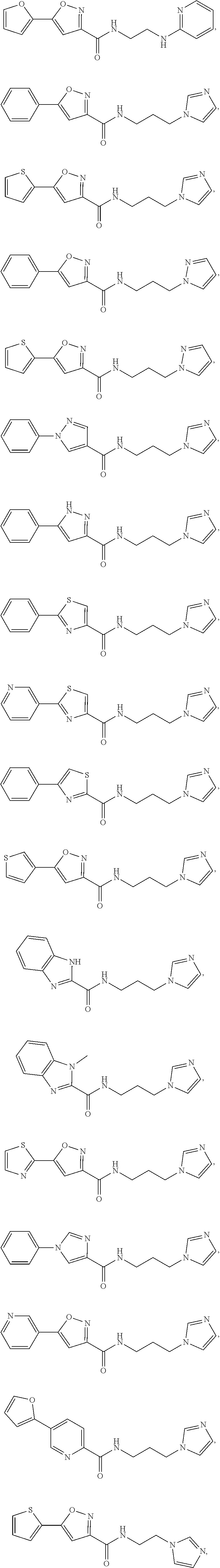

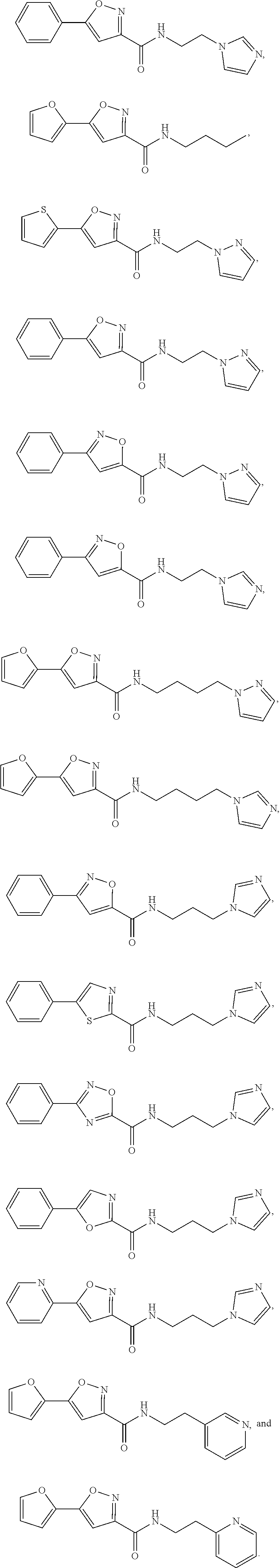

14. The method of claim 1, wherein the agent that inhibits Mnk1/2 is selected from the group consisting of CGP-57380, cercosporamide, BAY 1143269, tomivosertib, ETC-206, SLV-2436 and any combination thereof.

15. The method of claim 1, wherein the agent that inhibits PI3K pathway is selected from the group consisting of 3-methyladenine, LY294002, alpelisib, wortmannin, quercetin, hSMG-1 inhibitor 11j, zandelisib, alpelisib hydrochloride, idelalisib, buparlisib, copanlisib, IP1549, dactolisib, pictilisib, SAR405, duvelisib, fimepinostat, GDC-0077, PI-103, YM-20163, PF-04691502, Taselisib, omipalisib, samotolisib, isorhamnetin, ZATK474, parsaclisib, rigosertib, AZD8186, GSK2636771, disitertide, TG100-115, AS-605240, PI3K-IN-1, dactolisib tosylate, gedatolisib, TGX-221, umbralisib, AZD 6482, serabelisib, bimiralisib, apitolisib, alpha-linolenic acid, Vps34-PIK-III, PIK-93, Vps34-IN-1, CH5132799, leniolisib, voxtalisib, GSK1059615, sonolisib, PKI-402, PI4KIIIbeta-IN-9, HS-173, BGT226 maleate, pictilisib dimethane sulfonate, VS-5584, IC-87114, quercetin dihydrate, CNX-1351, SF2523, GDC-0326, seletalisib, acalisib, SAR-260301, ZAD-8835, GNE-317, AMG319, nemiralisib, IITZ-01, PI-103 hydrochloride, oroxin B, pilaralisib, AS-252424, cpanlisib dihydrochloride, AMG 511, disitertide TFA, PIK-90, tenalisib, esculetin, CGS 15943, GNE-477, PI-3065, A66, AZD3458, ginsenoside Rk1, sophocarpine, buparlisib hydrochloride, Vps34-IN-2, linperlisib, arnicolide D, KP372-1, CZC24832, PF-4989216, (R)-Duvelisib, PQR530, P115-IN-1, umbralisib hydrochloride, MTX-211, PI3K/mTOR Inhibitor-2, LX2343, PF-04979064, polygalasaponin F, glaucocalyxin A, NSC781406, MSC2360844, CAY10505, IPI-3063, TG 100713, BEBT-908, PI-828, brevianamide F, ETP-46321, PIK-294, SRX3207, sophocarpine monohydrate, AS-604850, desmethylglycitein, SKI V, WYE-687, NVP-QAV-572, GNE-493, CAL-130 hydrochloride, GS-9901, BGT226, IHMT-PI3Kδ-372, PI3Kα-IN-4, parsaclisib hydrochloride, PF-06843195, PI3K-IN-6, (S)-PI3Kα-IN-4, PI3K(gamma)-IN-8, BAY1082439, CYH33, PI3Kγ inhibitor 2, PI3Kδ inhibitor 1, PARP/PI3K-IN-1, LAS191954, PI3K-IN-9, CHMFL-PI3KD-317, PI3K/HDAC-IN-1, MSC2360844 hemifumarate, PI3K-IN-2, PI3K/mTOR Inhibitor-1, PI3Kδ-IN-1, euscaphic acid, KU-0060648, AZD 6482, WYE-687 dihydrochloride, GSK2292767, (R)-Umbralisib, PIK-293, idelalisib D5, PIK-75, hirsutenone, quercetin D5, PIK-108, hSMG-1 inhibitor 11e, PI3K-IN-10, NVP-BAG956, PI3Kγ inhibitor 1, CAL-130, ON 146040, PI3kδ inhibitor 1, PI3Kα/mTOR-IN-1, and any combination thereof.

16. A method to genetically modify granulocyte/macrophage progenitor (GMPs) cells, comprising:
genetically engineering a modification into GMPs made by the method of claim 1, using a gene editing system, homologous recombination, or site directed mutagenesis.

17. The method of claim 16, wherein the genetically engineering modification comprises replacing or disrupting an existing gene, or altering a genetic locus to contain sequence information not found at the genetic locus.

18. The method of claim 17, wherein the genetically engineering modification of the GMPs comprises a knockout SIRPα and/or PI3Kγ gene.

19. The method of claim 16, further comprising differentiating the GMPs into macrophages comprising:
culturing the GMPs with a macrophage differentiation medium comprising macrophage colony-stimulating factor (MCSF).

20. (canceled)

21. The method of claim 16, further comprising differentiating the GMPs into granulocytes comprising:
culturing the GMPs with a granulocyte differentiation medium comprising granulocyte colony-stimulating factor (GCSF).

22. (canceled)

23. A population of granulocyte/macrophage progenitor cells (GMPs) expanded by a method of claim 1.

24. Genetically modified granulocyte/macrophage progenitor cells (GMPs) prepared by a method of claim 16.

25. Macrophages prepared by a method of claim 19.

26. Granulocytes prepared by a method of claim 21.

27. A compound having the structure of Formula I: 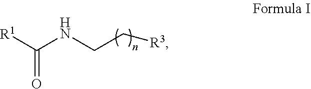
wherein,
R¹ is selected from: 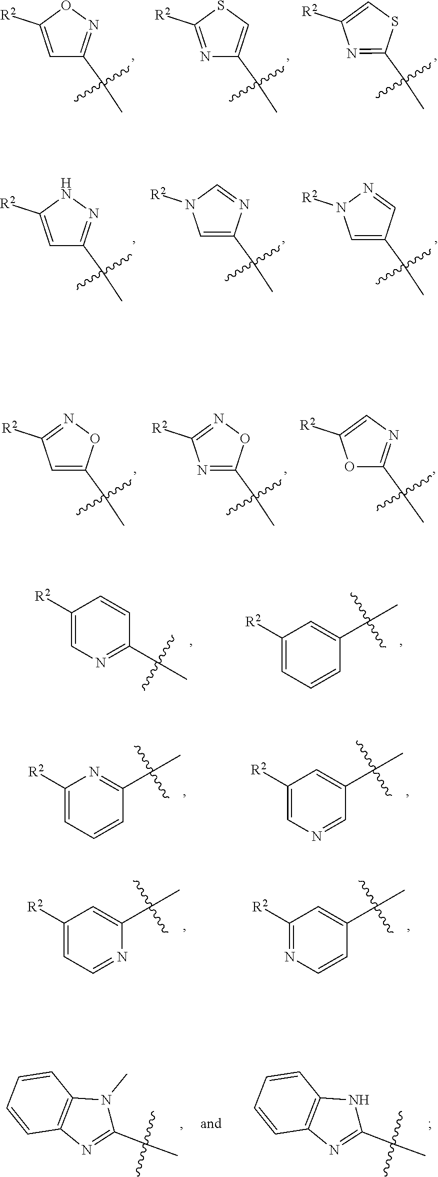
R² is selected from: 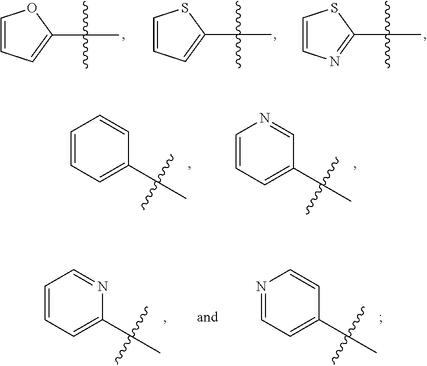
R³ is selected from: 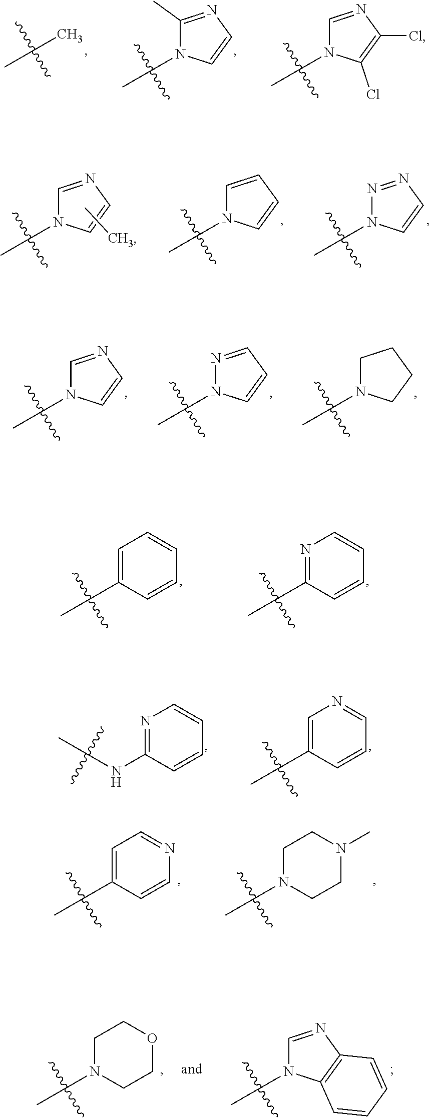
n is an integer selected from 0, 1, 2, 3, 4, and 5. In a further embodiment, a compound having the structure of Formula I is not

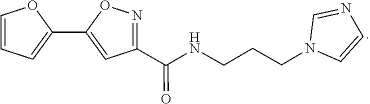 [SMILES: O=C(NCCCn1ccnc1)c1cc(-c2ccco2)on1]

28. The compound of claim 27, wherein the compound has a structure selected from the group consisting of:

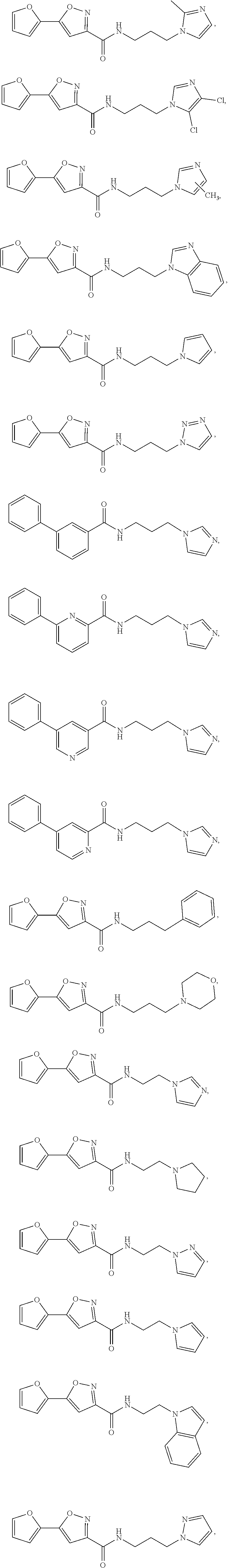

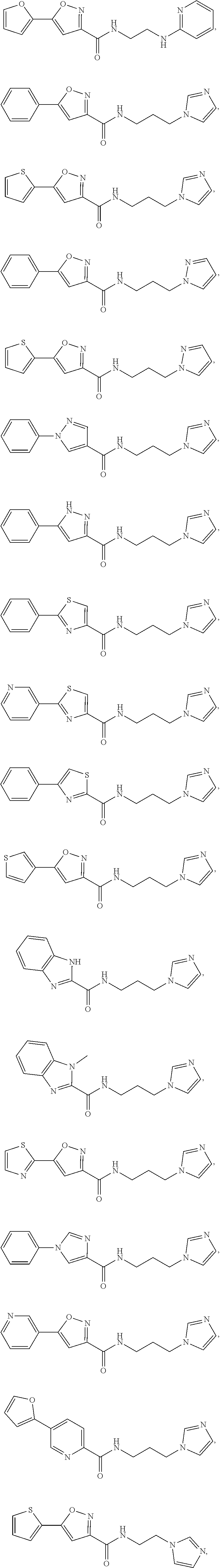

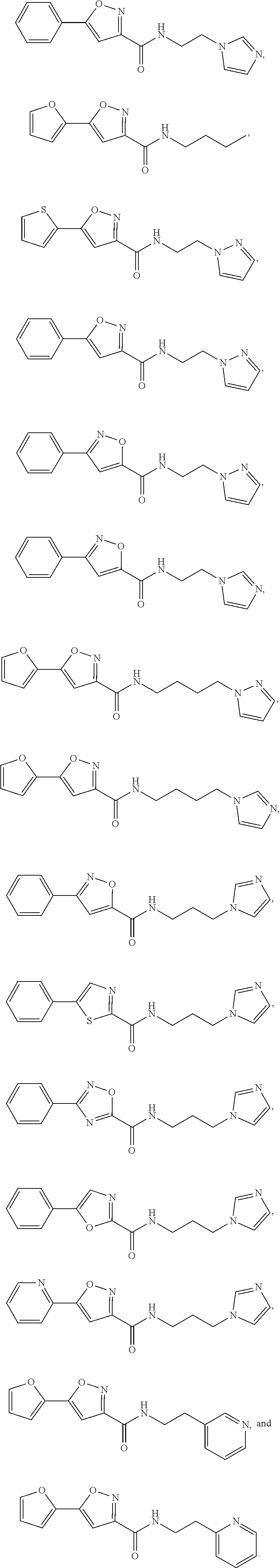

29. A cell culture medium comprising a compound having the structure of Formula I: 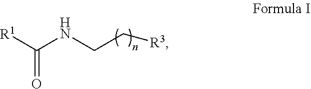
wherein,
R¹ is selected from: 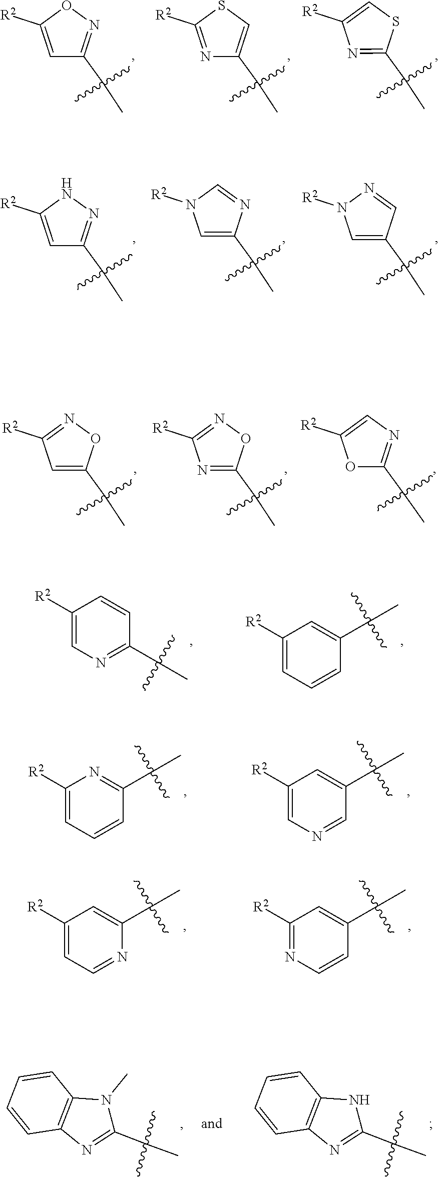
R² is selected from: 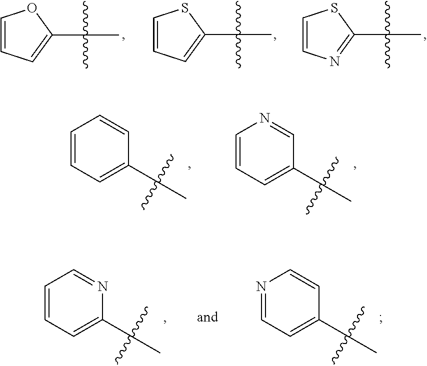
R³ is selected from: 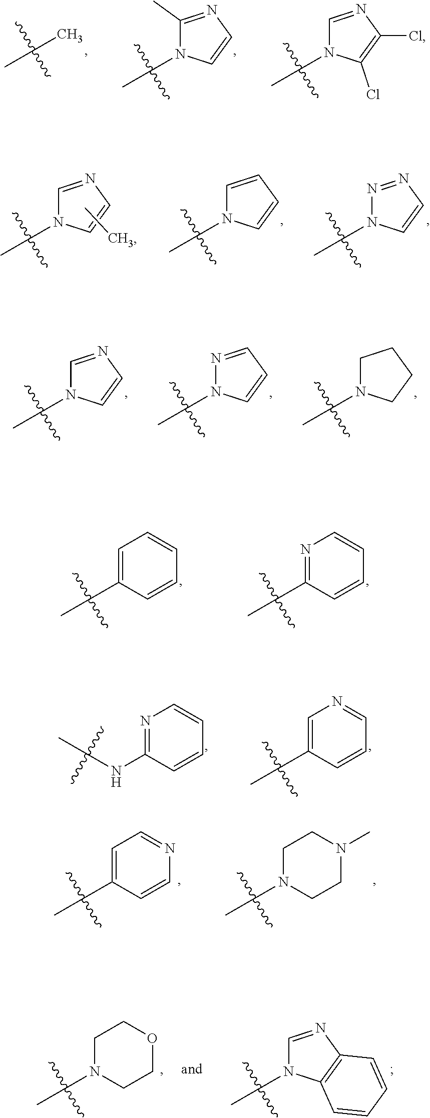
n is an integer selected from 0, 1, 2, 3, 4, and 5. In a further embodiment, a compound having the structure of Formula I is not

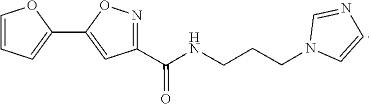 [SMILES: O=C(NCCCn1ccnc1)c1cc(-c2ccco2)on1]

30. The cell culture medium of claim 29, wherein the compound has a structure selected from the group consisting of: 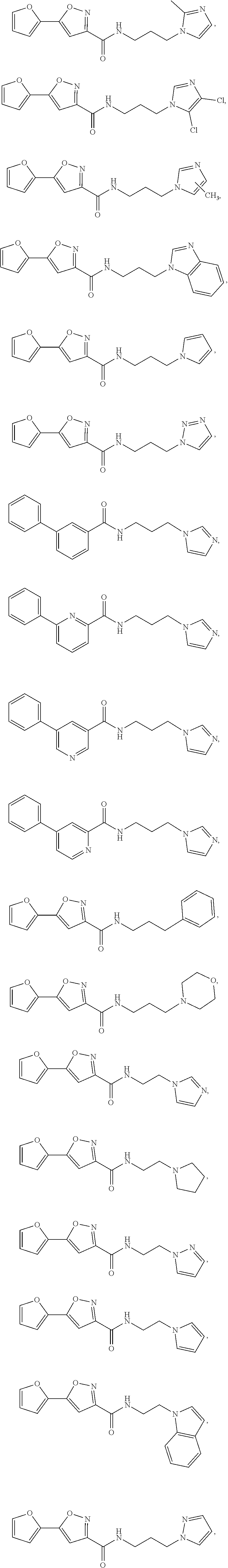 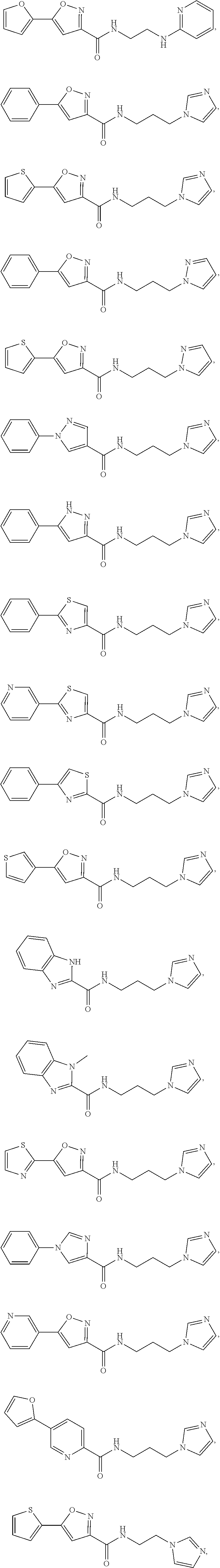 [chemical structure]
